(19) United States
(12) Patent Application Publication
(10) Pub. No.: US 2003/0219768 A1
(43) Pub. Date: Nov. 27, 2003

(54) Lung cancer therapeutics and diagnostics

(76) Inventors: Jean S. Beebe, Salem, CT (US); Kevin G. Coleman, Old Lyme, CT (US); Ethan Dmitrovsky, Hanover, NH (US); Thomas G. Turi, Old Saybrook, CT (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/286,989

(22) Filed: Nov. 2, 2002

Related U.S. Application Data
(60) Provisional application No. 60/336,024, filed on Nov. 2, 2001.
(60) Provisional application No. 60/335,317, filed on Nov. 2, 2001.
(60) Provisional application No. 60/336,298, filed on Nov. 2, 2001.

(51) Int. Cl. C12Q 1/68; G01N 33/53; G01N 33/574; G01N 33/543

(52) U.S. Cl. 435/6; 435/7.1; 435/7.23; 436/518

(57) ABSTRACT

The present invention provides genes that are differentially expressed during neoplasia. These genes and gene products comprise panels for use in screening candidate agents for therapeutic intervention in lung cancers, and for use in therapeutic, prognostic and diagnostic methods and compositions. Therapeutic agents are also provided by the invention. Diagnostic compositions include compositions comprising detection agents for detecting one or more genes that have been shown to be up-or down-regulated in pathogenesis of lung cancer. Exemplary detection agents include nucleic acid probes, which can be in solution or attached to a solid surface, e.g., in the form of a microarray. The invention also provides computer-readable media comprising values of levels of expression of one or more genes that are modulated in lung cancer.

### RELATED APPLICATION INFORMATION

This application claims the benefit of priority to the following U.S. Provisional Patent Applications, all of which applications are hereby incorporated by reference in their entireties: U.S. S No. 60/336,024; U.S. S No. 60/335,317; and U.S. S No. 60/336,298; all filed on Nov. 2, 2001.

### BACKGROUND OF THE INVENTION

Lung cancer is the leading cause of cancer death in both men and women in Western society. If lung cancer is found and treated early, before it has spread to lymph nodes or other organs, the five-year survival rate is about 42%. However, few lung cancers are found at this early stage. The five-year survival rate for all stages of lung cancer combined was 14% in 1995, the last year for which national data is available. Since most people with early lung cancer do not have any symptoms, only about 15% of lung cancers are found in the early stages. There are two major types of lung cancer. The first is non-small cell lung cancer. The other is small cell lung cancer. If the cancer has features of both types, it is called mixed small cell/large cell cancer.

Non-small cell lung cancer (NSCLC) is the most common type of lung cancer, accounting for almost 80% of lung cancers. Risk factors for NSCLC include prior smoking, passive smoking, and radon exposure. The main types of NSCLC are squamous cell carcinoma (also called epidermoid carcinoma), adenocarcinoma, bronchoalveolar carcinoma, large cell carcinoma, adenosquamous carcinoma, and undifferentiated carcinoma. Squamous cell carcinoma forms in cells lining the airways. Adenocarcinoma is the most common type of non-small cell lung cancer and is the form that often occurs in people who have never smoked, and begins in the mucus-producing cells of the lung.

Lung cancer is best treated when it is diagnosed early. However, most patients are not diagnosed until they exhibit symptoms. Symptoms of lung cancer include cough or chest pain, a wheezing sound when breathing, shortness of breath, coughing up blood, hoarseness, or swelling in the face and neck. When a patient exhibits symptoms of lung cancer, a bronchoscopy is performed so that cells from the walls of the bronchial tubes may be examined and small pieces of tissue removed for biopsy. If the suspect tissue is unable to be obtained through this method, needle aspiration biopsy may be performed in which a needle inserted between the ribs to draw cells from the lung, or surgery is performed to remove tissue for biopsy. Diagnosis of cancer is made by examination of the characteristics of the cells under a microscope.

The following stages are used for classifying lung cancer:

Occult stage: Cancer cells are found in sputum, but no tumor can be found in the lung.

Stage 0: Cancer is only found in a local area and only in a few layers of cells. It has not grown through the top lining of the lung. Another term for this type of cell lung cancer is carcinoma in situ.

Stages I & II For a description, see a standard textbook in the field, e.g., DeVita et al., Principles and Practices of Oncology, 5^{th} Edition, Lippincolt-Ravey, pp. 858-911

Stage III: Cancer has spread to the chest wall or diaphragm near the lung; or the cancer has spread to the lymph nodes in the area that separates the two lungs (mediastinum); or to the lymph nodes on the other side of the chest or in the neck. Stage III is further divided into stage IIIA (usually may be operated upon) and stage IIIB (usually may not be operated on).

Stage IV: Cancer has spread to other parts of the body.

Recurrent: Cancer has come back (recurred) after previous treatment.

Treatment for lung cancer depends on the stage of the disease, the age of the patient, and the overall condition of the patient. Patients may be divided into three groups, depending on the stage of the cancer and the treatment that is planned. The first group (stages 0, I, and II) includes patients whose cancers can be taken out by surgery. The second group (stage III) of patients has lung cancer that has spread to nearby tissue or to mediastinal or supraclavicular lymph nodes. These patients may be treated with radiation therapy alone or with surgery and radiation, chemotherapy and radiation, or chemotherapy alone. The group of patients with most advanced lung cancers (stage IV) are generally treated with chemotherapy alone, or a combination of chemotherapy and radiation therapy. Surgery generally is not a treatment option for Stage IV lung cancer. The most effective treatment is chemotherapy, either alone or in combination with radiation therapy. The exact treatment depends on the extent of the cancer (limited or extensive stage).

Surgery, chemotherapy and radiation have moderate to severe side effects, particularly when a mid- to late-stage cancer is being treated and the treatment is more aggressive. Surgery for lung cancer is a major operation. After lung surgery, air and fluid collect in the chest. Patients often need help turning over, coughing, and breathing deeply to expand the remaining lung tissue and get rid of excess air and fluid. Pain or weakness in the chest and the arm and shortness of breath are common side effects of cancer surgery, and may be chronic side effects in cases where all or part of a lung is removed. Patients may need several weeks or months to regain their energy and strength. Chemotherapy works by preventing cells from growing and dividing. The effect is strongest on very rapidly dividing cells, such as cancer cells, but normal tissues may also be affected, particularly the bone marrow, the gastrointestinal or GI tract, the reproductive system, and hair follicles. This may manifest itself in such ways as fatigue, mouth sores, nausea, hair loss, anemia, immunosuppression, and reproductive problems. Radiation therapy works by locally destroying cancerous tissue. Local side effects result from damage to the surrounding tissue, such as burns or hair loss. General side effects may also result from radiation therapy, however, and are similar to those from chemotherapy. Side effects associated with cancer treatment could be ameliorated if more genes associated with tumor development, progression, and maintenance could be identified and their expression regulated by novel therapies. An ideal target would comprise a gene that is expressed at low levels or not at all in normal cells that is expressed at high levels during tumorigenesis. A therapeutic directed at such a target would have the greatest effect on the tumor cells, with little or no effect on normal cells, ameliorating toxic side effects.

Ideally, the use of aggressive chemotherapy, radiation, and surgical treatment regimens could be rendered unnecessary by early diagnosis or detection of lung cancer. Lung cancer is usually asymptomatic until it has reached an advanced stage. No effective diagnostic exists for individuals in whom symptoms have not appeared. The chest radiograph (x-ray) and sputum cytomorphologic examination (cytology) lack sufficient accuracy to be used in routine screening of asymptomatic persons. The accuracy of the chest x-ray is limited by the capabilities of the technology and observer variation among radiologists. Suboptimal technique, insufficient exposure, and poor positioning and cooperation of the patient may obscure pulmonary nodules or introduce artifacts. Sputum cytology is an even less effective screening test, largely due to its low sensitivity compared to chest x-ray. In summary, there is no good evidence that screening for lung cancer can reduce lung cancer mortality. Screening with chest x-ray plus sputum cytology appears to detect lung cancer at an earlier stage, but this would be expected in a screening test whether or not it was effective at reducing mortality. Currently, the National Institutes of Health do not recommend routine screening for lung cancer with chest radiography or sputum cytology in asymptomatic persons, rather, it recommends that all patients should be counseled against tobacco use to prevent cancer in the first place. A more sensitive technique that requires a small sample of cells would provide a better diagnostic, such as one which takes advantage of current molecular biological techniques, such as, for example, current spiral computed tomography (CT) technology, which may represent a technical advance for lung cancer screening through its improved imaging approach.

### SUMMARY OF THE INVENTION

The present invention relates to novel genes and/or the encoded gene products identified by gene expression profiling as being differentially expressed during neoplasia of lung cells. The present invention also relates to novel panels of molecular targets comprised of genes or groups of genes that are differentially regulated during neoplasia of lung cells and were discovered using microarray technology and gene expression profiling of both normal and cancerous lung tissue, as described e.g., in the Examples and shown in the FIGURES. Based on this identification, the invention features in one aspect an expression profile, hereafter referred to as a “panel”, of these genes and/or encoded gene products.

In one embodiment, the panel is comprised of at least one gene and/or encoded gene product selected from the group of genes listed in FIG. 2 that are differentially regulated during pathogenesis of lung tumor cells. In certain embodiments, the panel is comprised of at least one gene and/or encoded gene product selected from the group of genes listed in FIG. 3 that are differentially regulated during pathogenesis of lung adenocarcinomas. In certain embodiments, the panel is comprised of at least one gene and/or encoded gene product selected from the group of genes listed in FIG. 4 that are differentially regulated during pathogenesis of lung squamous cell carcinomas.

The present invention also relates to TrkB (e.g., NCBI Reference Sequence project (“RefSeq”) and GenBank Accession number U12140) and/or its encoded gene product, which was identified by gene expression profiling as being differentially expressed during neoplasia of lung cells. The present invention further relates to the use of this gene or its gene products in methods of identifying candidate therapeutic agents for use in early intervention in lung cancer. In such embodiments, the TrkB gene and/or its encoded gene products comprise the “panel” for these methods. In some embodiments, candidate therapeutic agents, or “therapeutics” are evaluated for their ability to bind a target protein.

The present invention also relates to Aur2 (e.g., RefSeq number NM_{—}003600, GenBank Accession numbers AF011468, AF008551, and BC001280) and/or its encoded gene product, which was identified by gene expression profiling as being differentially expressed during neoplasia of lung cells. The present invention further relates to the use of this gene or its gene products in methods of identifying candidate therapeutic agents for use in early intervention in lung cancer. In such embodiments, the Aur2 gene and/or its encoded gene products comprise the “panel” for these methods. In some embodiments, candidate therapeutic agents, or “therapeutics” are evaluated for their ability to bind a target protein.

The present invention further relates to the use of the panels in methods of identifying candidate therapeutic agents for use in early intervention in lung cancer. In one embodiment of the invention, the cancer is adenocarcinoma and the panel comprises at least one gene and/or encoded gene product of FIG. 3. In another embodiment of the invention, the cancer is squamous cell carcinoma and the panel comprises at least one gene and/or encoded gene product of FIG. 4. Individual genes or groups of genes in the panels of the present invention, and their encoded gene products, comprise the “targets” for these methods. In one embodiment, the “target” for these methods is the TrkB gene or gene product. In another embodiment, the “target for these methods is the Aur2 gene or gene product. In some embodiments, candidate therapeutic agents, or “therapeutics” are evaluated for their ability to bind a target protein. The candidate therapeutics may be selected, for example, from the following classes of compounds: proteins including antibodies, peptides, peptidomimetics, or small molecules. In other embodiments, candidate therapeutics are evaluated for their ability to bind a target gene. The candidate therapeutics may be selected, for example, from the following classes of compounds: antisense nucleic acids, small molecules, polypeptides, proteins, including antibodies, peptidomimetics, or nucleic acid analogs. In any of the embodiments, the candidate therapeutics may be selected from a library of compounds. These libraries may be generated using combinatorial synthetic methods.

The ability of said candidate therapeutics to bind a target molecule comprising a panel of the present invention may be determined using a variety of suitable assays known to those of skill in the art. In certain embodiments of the present invention, the ability of a candidate therapeutic to bind a target protein or gene may be evaluated by an in vitro assay. In either embodiment, the binding assay may also be an in vivo assay.

The present invention further provides methods for evaluating candidate therapeutic agents of the present invention for their ability to modulate the expression of a target gene by contacting the lung cells of a subject with said candidate therapeutic agents. In certain embodiments, the candidate therapeutic will be evaluated for its ability to normalize the expression levels of a gene or group of genes. Alternatively, candidate therapeutic agents may be evaluated for their ability to inhibit the activity of a protein that promotes lung cell pathogenesis by contacting the lung cells of a subject with said candidate therapeutic agents and evaluating its ability to inhibit the activity of said protein.

Assays and methods of developing assays suitable for use in the methods described above are known to those of skill in the art and, as will be appreciated by those skilled in the art, based upon the present description, may be used as suitable with the methods of the present invention.

The present invention provides methods for determining the efficacy of a candidate therapeutic as a drug for lung cancer. In one embodiment, methods for determining efficacy may comprise the steps of a) contacting a candidate therapeutic to a lung tumor cell of a subject; and b) determining the ability of said candidate therapeutic to inhibit pathogenesis of the cell. In another embodiment, a method for determining efficacy may comprise the steps of a) contacting a candidate therapeutic to a lung tumor cell of a subject; and b) determining the ability of said candidate therapeutic to normalize the expression profile of said cell. Alternatively, candidate therapeutics may be screened for efficacy by comparing the expression level of one or more genes associated with lung cell neoplasia after incubating a cell of a subject having lung cancer or similar cell, such as one in a preneoplastic lesion, with the candidate therapeutic. In an even more preferred embodiment, the expression level of the genes is determined using microarrays or other methods of RNA quantitation, and by comparing the gene expression profile of a cell in response to the test compound with the gene expression profile of a normal cell corresponding to a cell of a subject having lung cancer or a preneoplastic lesion (a “reference profile”).

Also within the scope of the invention are pharmaceutical compositions, e.g., compositions comprising therapeutic agents identified by the methods described herein together with a pharmaceutically-acceptable carrier, vehicle, or diluent, and methods of therapy using these compositions. In certain embodiments, the pharmaceutical compositions of the invention are used to treat patients with adenocarcinoma. In other embodiments, the pharmaceutical compositions are used to treat patients with squamous cell carcinoma, or other types of non-small cell lung cancer, as well as preneoplastic lesions. In still other embodiments, the pharmaceutical compositions may be used in a preventative method in a subject who has had or may be at risk of developing lung cancer. The present invention further provides the use of pharmaceutical compositions to modulate the activity of a protein in the lung cells of a subject with lung cancer and return the activity to a level found in a normal subject. The present invention also provides the use of pharmaceutical compositions to modulate the expression levels of a gene in the lung cells of a subject with lung cancer and return the expression levels to a level found in a normal subject. The present invention also provides the use of pharmaceutical compositions to kill malignant lung cells. Such methods may include administering to a subject having lung cancer a pharmaceutically-efficient amount of a modulator (e.g., an agonist or antagonist) of one or more genes or their encoded proteins involved in regulation of lung cancer. Compositions for up-regulating the expression of genes which are down-regulated in lung cancer include polypeptides, or functional fragments thereof, that are encoded by genes characteristic of lung cancer; nucleic acids encoding these; and compounds identified as up-regulating the expression or activity of the polypeptides. Compositions for down-regulating the expression of genes which are up-regulated in lung cancer include, for example, antisense nucleic acids; ribozymes; small interfering RNAs (siRNAs); dominant negative mutants of polypeptides encoded by the genes and nucleic acids encoding such; antibodies that recognize the polypeptides encoded by the genes; and compounds identified as down-regulating the expression or activity of the polypeptides. In an alternative embodiment of the present invention, methods of treating a subject having lung cancer comprise, for example, administering to said subject a protein encoded by the panels of the present invention whose levels are deficient during lung cell pathogenesis.

In another aspect, the invention provides diagnostic methods for monitoring the existence and/or evolution of lung cancer in a subject. For example, the invention provides methods for predicting whether a subject is likely to develop lung cancer; methods for confirming that a subject, who has been diagnosed as having lung cancer with traditional methods, has lung cancer, and not, e.g., a disease that is phenotypically related to lung cancer; and methods for monitoring the progression of the disease, e.g., in a subject undergoing treatment. Preferred methods comprise determining the level of expression of one or more genes whose expression is characteristic of lung cancer in the lung cells of a subject. Other methods comprise determining the level of expression of tens, hundreds or thousands of genes whose expression is characteristic of lung cancer, e.g., by using microarray technology. The expression levels of the genes are then compared to the expression levels of the same genes of one or more other cells, e.g., a normal cell, or a diseased lung cell.

Comparison of the expression levels may be performed visually. In a preferred embodiment, the comparison is performed by a computer. In one embodiment, expression levels of genes whose expression is characteristic of lung cancer in cells of subjects having lung cancer are stored in a computer. The computer may optionally comprise expression levels of these genes in normal cells. The data representing expression levels of the genes in a patient being diagnosed are then entered into the computer, and compared with one or more of the expression levels stored in the computer. The computer calculates differences and presents data showing the differences in expression of the genes in the two types of cells.

In one embodiment, a cell sample from a patient is obtained, the level of expression of one or more genes whose expression is characteristic of lung cancer is determined, the expression data are entered into a computer comprising a plurality of reference expression data associated with particular therapies and compared thereto, to determine the most suitable therapy for the patient. The method may further optionally comprise sending, e.g., to a caregiver, the identity of the suitable therapy. The data and identity of the suitable therapy may be sent via a network, e.g., the internet.

In other embodiments of the diagnostic methods provided by the present invention, a method of diagnosis may comprise (a) determining the activity of a protein encoded by a gene selected from the panels of the invention in a lung cell of a subject, and (b) comparing the activity of said protein in said subject's cell with that of a normal lung cell of the same type. In certain embodiments, a particular type of lung cancer may be diagnosed if the protein whose activity is determined is associated with a particular type of lung cancer, such as adenocarcinoma or squamous cell carcinoma.

The invention also provides compositions comprising one or more detection agents for detecting the expression of genes whose expression is characteristic of lung cancer, e.g., for use in diagnostic assays. These agents, which may be, e.g., nucleic acids or polypeptides, maybe in solution or bound to a solid surface, such as in the form of a microarray. Other embodiments of the invention include databases, computer readable media, computers containing the gene expression profile[s] of the invention or the level of expression of one or more genes whose expression is characteristic of lung cancer in a diseased lung cell.

The present invention further provides a kit comprising a plurality of gene expression patterns and reagents for determining gene expression levels. To give but one example, the expression level may be determined by providing a kit containing suitable reagents and an suitable microarray for determining the level of expression in the lung cells of a subject. In other embodiments, the invention provides a kit including compositions of the present invention. Any of the above-described kits may comprise instructions for their use. Such kits may have a variety of uses, including, for example, imaging, diagnosis, and therapy.

These embodiments of the present invention, other embodiments, and their features and characteristics will be even more apparent from the description, drawings, and claims that follow.

### BRIEF DESCRIPTION OF THE DRAWINGS

FIG. 1 shows a schematic of an informatics approach that may be used in the present invention for selecting novel targets from genes that exhibited differential expression in lung cell neoplasia.

FIG. 2 lists genes that were determined to be differentially expressed during pathogenesis of lung cells.

FIG. 3 lists genes that were determined to be differentially expressed during pathogenesis of lung adenocarcinomas.

FIG. 4 lists genes that were determined to be differentially expressed during pathogenesis of lung squamous cell cancers

### DETAILED DESCRIPTION OF THE INVENTION

1. General

The panels of the invention were provided via analysis of differential gene expression by microarray in a library of 39 individual clinical samples. The library was generated from surgically resected clinical samples representing individual tumorous or normal lung tissue samples derived from biopsy material. The library was comprised of tumor tissue samples derived from 24 lung tumor samples comprising both adenocarcinoma and squamous cell carcinoma at all stages (occult, stage I-IV, and recurrent), one neuroendocrine tumor, one bronchioalveolar, one large cell tumor, and 13 normal lung tissue samples. Of these samples, 8 were “matched-pairs”, in that for a given tumor tissue sample, normal tissue from the same individual was also obtained. Differential gene expression during tumor development was characterized in the samples by analyzing the gene expression profiles of the same type of lung cancer at multiple stages of development.

Analysis of gene expression profiles of the samples was accomplished using a custom Affymetrix GeneChip® (Santa Clara, Calif.) designed to include a subset of the human genome based on a variety of criteria. The genes were selected from the Incyte GeneAlbum® (Palo Alto, Calif.) database. The clinical lung tissue samples representing distinct tumor types and normal samples were interrogated using the GeneChip and a gene expression profile created for each sample. This was performed using standard Affymetrix methods (Mahadevappa, M. and Warrington, J. A., (1999) Nat. Biotechnol, 17:1134-1136). Briefly, mRNA was isolated from normal and tumor samples, cRNA was made from the mRNA and hybridized to the chip, which was analyzed to identify genes that are regulated during development, progression, or maintenance of the tumor.

The function and biological activity of the 1000 or more genes identified as being differentially regulated between normal and tumor samples were identified through a database that links genes sequences to biochemical pathways, e.g., see the Kyoto Encyclopedia of Genes and Genomes (KEGG) from Kyoto University and/or the PFBP database consortium sponsored by the European Bioinformatics Institute (EBI). A smaller subset of genes were selected from this pool of genes based on criteria described more thoroughly in the Exemplification.

2. Definitions

For convenience, before further description of the present invention, certain terms employed in the specification, examples and appended claims are defined here.

The singular forms “a”, “an”, and “the” include plural references unless the context clearly dictates otherwise.

An “address” on an array, e.g., a microarray, refers to a location at which an element, e.g., an oligonucleotide, is attached to the solid surface of the array. As used herein, a nucleic acid or other molecule attached to an array, is referred to as a “probe” or “capture probe.” When an array contains several probes corresponding to one gene, these probes are referred to as “gene-probe set.” A gene-probe set may consist of, e.g., 2 to 10 probes, preferably from 2 to 5 probes and most preferably about 5 probes.

“Adenocarcinoma” refers to cancer whose point of origin was in any glandular cell, or adeno cell. “Adenocarcinoma of the lung” refers to a cancer of the mucous-producing cells of the lungs.

“Agonist” refers to an agent that mimics or up-regulates (e.g., potentiates or supplements) the bioactivity of a protein, e.g., polypeptide X. An agonist may be a wild-type protein or derivative thereof having at least one bioactivity of the wild-type protein. An agonist may also be a compound that upregulates expression of a gene or which increases at least one bioactivity of a protein. An agonist may also be a compound which increases the interaction of a polypeptide with another molecule, e.g., a target peptide or nucleic acid.

“Allele”, which is used interchangeably herein with “allelic variant”, refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene may differ from each other in a single nucleotide, or several nucleotides, and may include substitutions, deletions, and insertions of nucleotides. An allele of a gene may also be a form of a gene containing a mutation.

“Amplification,” refers to the production of additional copies of a nucleic acid sequence. Amplification is generally carried out using polymerase chain reaction (PCR) technologies well known in the art. (Dieffenbach, C. W. and G. S. Dveksler (1995) PCR Primer, a Laboratory Manual, Cold Spring Harbor Press, Plainview, N.Y.)

“Antagonist” refers to an agent that downregulates (e.g., suppresses or inhibits) at least one bioactivity of a protein. An antagonist may be a compound which inhibits or decreases the interaction between a protein and another molecule, e.g., a target peptide or enzyme substrate. An antagonist may also be a compound that downregulates expression of a gene or which reduces the amount of expressed protein present.

“Antibody” is intended to include whole antibodies, e.g., of any isotype (IgG, IgA, IgM, IgE, etc.), and includes fragments thereof which are also specifically reactive with a vertebrate, e.g., mammalian, protein. Antibodies may be fragmented using conventional techniques and the fragments screened for utility in the same manner as described above for whole antibodies. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The subject invention includes polyclonal, monoclonal, humanized, or other purified preparations of antibodies and recombinant antibodies.

“Antisense” nucleic acid refers to oligonucleotides which specifically hybridize (e.g., bind) under cellular conditions with a gene sequence, such as at the cellular mRNA and/or genomic DNA level, so as to inhibit expression of that gene, e.g., by inhibiting transcription and/or translation. The binding may be by conventional base pair complementarily, or, for example, in the case of binding to DNA duplexes, through specific interactions in the major groove of the double helix.

“Array” or “matrix” refer to an arrangement of addressable locations or “addresses” on a device. The locations may be arranged in two dimensional arrays, three dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes. The nucleic acid on the array is preferably single stranded. Arrays wherein the probes are oligonucleotides are referred to as “oligonucelotide arrays” or “oligonucleotide chips” or “gene chips”. A “microarray”, also referred to as a “chip”, “biochip”, or “biological chip”, is an array of regions having a suitable density of discrete regions, e.g., of at least 100/cm², and preferably at least about 1000/cm². The regions in a microarray have dimensions, e.g. diameters, preferably in the range of between about 10-250 microns, and are separated from other regions in the array by the same distance.

“Biological activity” or “bioactivity” or “activity” or “biological function”, which are used interchangeably, refer to an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity may be modulated by directly affecting the subject polypeptide. Alternatively, a bioactivity may be altered by modulating the level of the polypeptide, such as by modulating expression of the corresponding gene.

“Biological sample” or “sample”, refers to a sample obtained from an organism or from components (e.g., cells) of an organism. The sample may be of any biological tissue or fluid. Frequently the sample will be a “clinical sample” which is a sample derived from a patient. Such samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes.

“Biomarker” refers to a biological molecule whose presence, concentration, activity, or post-translationally-modified state may be detected and correlated with the activity of a protein of interest.

“Cell cycle” refers to a repeating sequence of events in eukaryotic cells consisting of two periods: first, a cell-growth period comprising the first gap or growth phase (G1), the DNA synthesis phase (S), and the second gap or growth phase (G2); and second, a cell-division period comprising mitosis (M).

“A corresponding normal cell of” or “normal cell corresponding to” or “normal counterpart cell of” a diseased cell refers to a normal cell of the same type as that of the diseased cell. “Diseased lung cell” refers to a malignant lung cell.

A “combinatorial library” or “library” is a plurality of compounds, which may be termed “members,” synthesized or otherwise prepared from one or more starting materials by employing either the same or different reactants or reaction conditions at each reaction in the library. In general, the members of any library show at least some structural diversity, which often results in chemical diversity. A library may have anywhere from two different members to about 10⁸ members or more. In certain embodiments, libraries of the present invention have more than about 12, 50 and 90 members. In certain embodiments of the present invention, the starting materials and certain of the reactants are the same, and chemical diversity in such libraries is achieved by varying at least one of the reactants or reaction conditions during the preparation of the library. Combinatorial libraries of the present invention may be prepared in solution or on the solid phase.

“Complementary” or “complementarity”, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands.

“Cytokine” refers to soluble biochemicals produced by cells that mediate reactions between cells, usually used for biological response modifiers.

A “delivery complex” refers to a targeting means (e.g., a molecule that results in higher affinity binding of a gene, protein, polypeptide or peptide to a target cell surface and/or increased cellular or nuclear uptake by a target cell). Examples of targeting means include: sterols (e.g., cholesterol), lipids (e.g., a cationic lipid, virosome or liposome), viruses (e.g., adenovirus, adeno-associated virus, and retrovirus) or target cell specific binding agents (e.g., ligands recognized by target cell specific receptors). Preferred complexes are sufficiently stable in vivo to prevent significant uncoupling prior to internalization by the target cell. However, the complex is cleavable under suitable conditions within the cell so that the gene, protein, polypeptide or peptide is released in a functional form.

“Derived from” as that phrase is used herein indicates a peptide or nucleotide sequence selected from within a given sequence. A peptide or nucleotide sequence derived from a named sequence may contain a small number of modifications relative to the parent sequence, in most cases representing deletion, replacement or insertion of less than about 15%, preferably less than about 10%, and in many cases less than about 5%, of amino acid residues or base pairs present in the parent sequence. In the case of DNAs, one DNA molecule is also considered to be derived from another if the two are capable of selectively hybridizing to one another.

“Derivative” refers to the chemical modification of a polypeptide sequence, or a polynucleotide sequence. Chemical modifications of a polynucleotide sequence may include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

“Detection agents of genes” refer to agents that may be used to specifically detect the gene or other biological molecule relating to it, e.g., RNA transcribed from the gene and polypeptides encoded by the gene. Exemplary detection agents are nucleic acid probes which hybridize to nucleic acids corresponding to the gene and antibodies.

“Differentiation” refers to the process by which a cell becomes specialized for a specific structure or function by selective gene expression of some genes and/or selective repression of others.

“Differential expression” refers to both quantitative as well as qualitative differences in a gene's temporal and/or tissue expression patterns. Differentially expressed genes may represent “target genes.”

“Differential gene expression pattern” between cell A and cell B refers to a pattern reflecting the differences in gene expression between cell A and cell B. A differential gene expression pattern may also be obtained, e.g., between a cell at one time point and a cell at another time point, or between a cell incubated or contacted with a compound and a cell that was not incubated with or contacted with the compound.

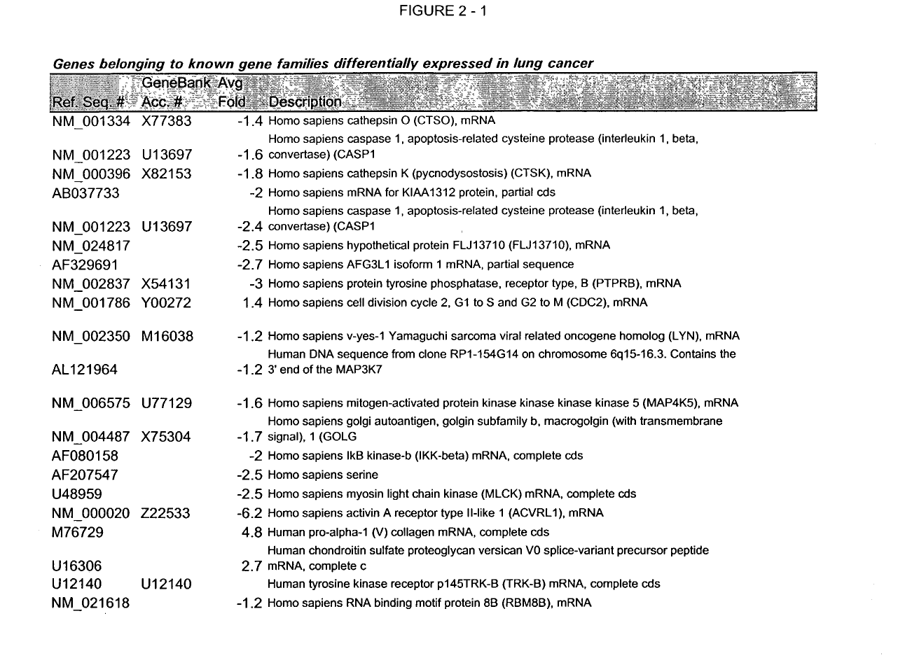

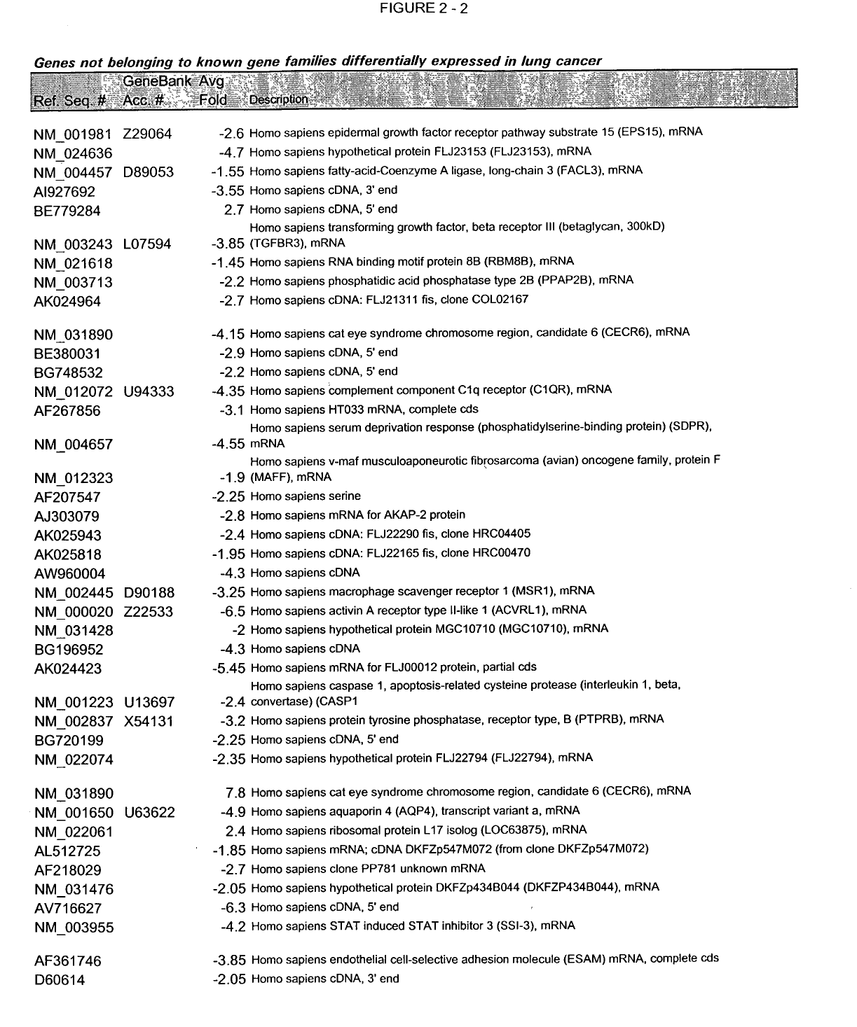

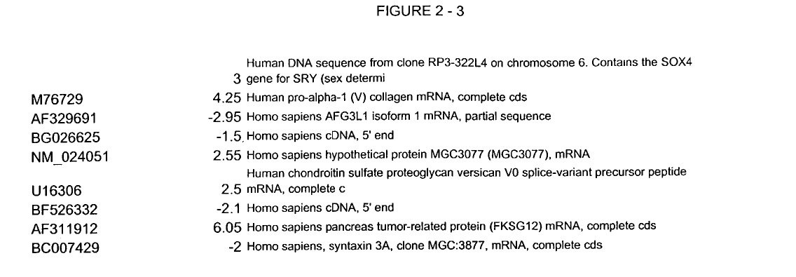

“Equivalent” refers to nucleotide sequences encoding functionally equivalent polypeptides. Equivalent nucleotide sequences will include sequences that differ by one or more nucleotide substitutions, additions or deletions, such as allelic variants; and will, therefore, include sequences that differ from the nucleotide sequence of the nucleic acids referred to in the FIGS. 2-4 due to the degeneracy of the genetic code.

“Expression profile,” which is used interchangeably herein with “gene expression profile” and “fingerprint” of a cell, refers to a set of values representing mRNA levels of a genes comprising the panels of the invention. An expression profile preferably comprises values representing expression levels of at least about 5 genes, preferably at least about 10, 25, 50, 100, 200 or more genes. Expression profiles preferably comprise an mRNA level of a gene which is expressed at similar levels in multiple cells and conditions. For example, an expression profile of a diseased cell of disease D refers to a set of values representing mRNA levels of 20 or more genes in a diseased cell.

The “level of expression of a gene in a cell” or “gene expression level” refers to the level of mRNA, as well as pre-mRNA nascent transcript(s), transcript processing intermediates, mature mRNA(s) and degradation products, encoded by the gene in the cell.

“Gene” or “recombinant gene” refer to a nucleic acid molecule comprising an open reading frame and including at least one exon and (optionally) an intron sequence. “Intron” refers to a DNA sequence present in a given gene which is spliced out during mRNA maturation.

“Gene construct” refers to a vector, plasmid, viral genome or the like which includes a “coding sequence” for a polypeptide or which is otherwise transcribable to a biologically active RNA (e.g., antisense, decoy, ribozyme, etc), may transfect cells, in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

“Heterozygote,” refers to an individual with different alleles at corresponding loci on homologous chromosomes. Accordingly, “heterozygous” describes an individual or strain having different allelic genes at one or more paired loci on homologous chromosomes.

“Homozygote,” refers to an individual with the same allele at corresponding loci on homologous chromosomes. Accordingly, “homozygous”, describes an individual or a strain having identical allelic genes at one or more paired loci on homologous chromosomes.

“Homology” or alternatively “identity” refers to sequence similarity between two peptides or between two nucleic acid molecules. Homology may be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The term “percent identical” refers to sequence identity between two amino acid sequences or between two nucleotide sequences. Identity may each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences.

As will be appreciated by one skill of art, particularly those in genomics or bioinformatics, various alignment algorithms and/or programs may be used or developed, including FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment, include, but are not limited to, those described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

“Hormone” refers to any one of a number of biochemical substances that are produced by a certain cell or tissue and that cause a specific biological change or activity to occur in another cell or tissue located elsewhere in the body.

“Host cell” refers to a cell transduced with a specified transfer vector. The cell is optionally selected from in vitro cells such as those derived from cell culture, ex vivo cells, such as those derived from an organism, and in vivo cells, such as those in an organism. “Recombinant host cells” refers to cells which have been transformed or transfected with vectors constructed using recombinant DNA techniques. “Host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Hybridization” refers to any process by which a strand of nucleic acid binds with a complementary strand through base pairing. “Specific hybridization” of a probe to a target site of a template nucleic acid refers to hybridization of the probe predominantly to the target, such that the hybridization signal may be clearly interpreted. As further described herein, such conditions resulting in specific hybridization vary depending on the length of the region of homology, the GC content of the region, and the melting temperature “T(m)” of the hybrid. Hybridization conditions will thus vary in the salt content, acidity, and temperature of the hybridization solution and the washes.

“Interact” is meant to include detectable interactions between molecules, such as may be detected using, for example, a hybridization assay. Interact also includes “binding” interactions between molecules. Interactions may be, for example, protein-protein, protein-nucleic acid, protein-small molecule or small molecule-nucleic acid in nature.

“Isolated”, with respect to nucleic acids, such as DNA or RNA, refers to molecules separated from other DNAs, or RNAs, respectively, that are present in the natural source of the macromolecule. Isolated also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. “Isolated” also refers to polypeptides which are isolated from other cellular proteins and is meant to encompass both purified and recombinant polypeptides.

“Label” and “detectable label” refer to a molecule capable of detection, including, but not limited to, radioactive isotopes, fluorophores, chemiluminescent moieties, enzymes, enzyme substrates, enzyme cofactors, enzyme inhibitors, dyes, metal ions, ligands (e.g., biotin or haptens) and the like. “Fluorophore” refers to a substance or a portion thereof which is capable of exhibiting fluorescence in the detectable range. Particular examples of labels which may be used under the invention include fluorescein, rhodamine, dansyl, umbelliferone, Texas red, luminol, NADPH, alpha- or beta-galactosidase and horseradish peroxidase.

“Lung cancer” refers in general to any malignant neoplasm found in the lung. The term as used herein encompasses both fully developed malignant neoplasms, as well as premalignant lesions. A “subject having lung cancer” is a subject who has a malignant neoplasm or premalignant lesion in the lungs.

A “molecular target” or “target” refers to a molecular structure that is a gene or derived from a gene that has been identified using the methods of the invention as exhibiting differential expression relative to another lung cell of interest. Exemplary targets as such are polypeptides, hormones, receptors, dsDNA fragments, carbohydrates or enzymes. Such targets also may be referred to as “target genes”, “target peptides”, “target proteins”, and the like.

“Modulation” refers to upregulation (i.e., activation or stimulation), downregulation (i.e., inhibition or suppression) of a response, or the two in combination or apart. A “modulator” is a compound or molecule that modulates, and may be, e.g., an agonist, antagonist, activator, stimulator, suppressor, or inhibitor.

“Neoplasia” refers to abnormal differentiation or maturation of tissue; a premalignant change characterized by alteration in the size, shape and organization of the cellular components of a tissue; or in general the loss in the uniformity of individual cells as well as in their architectural orientation. Neoplasia may be generally used to refer to any alteration that carries with it the potential of development of cancer.

“Neoplasm” refers to spontaneous new growth of tissue originating from normal cell that forms an abnormal mass. A neoplasm, which is an art-recognized synonym of the term “tumor”, serves no useful function and grows at the expense of the healthy organism. “Malignant neoplasm” refers to a neoplasm that is characterized by reduced control over growth and function leading to serious adverse effects on the host through invasive growth and metastasis. “Metastasis” refers to the spread of a malignant neoplasm from its original site to other areas in the body. “Cancer” refers in general to any malignant neoplasm or premalignant lesion. “Tumorigenesis” refers to the biological processes and cellular stages through which a tumor is formed from normal cells. “Pathogenesis of lung cells” or “pathogenesis of lung cancer” refer to the process of tumorigenesis in lung cells, as well as the process of metastasis e.g., all stages in the progression of lung cancer.

“Non-small cell lung cancer” refers to a cancer whose origin is in any of the cells of the lung except for those which are dedicated hormone-producing cells (e.g., the “small cells”).

“Normalizing expression of a gene” in a diseased cell refers to a means for compensating for the altered expression of the gene in the diseased cell, so that it is essentially expressed at the same level as in the corresponding non diseased cell. For example, where the gene is overexpressed in the diseased cell, normalization of its expression in the diseased cell refers to treating the diseased cell in such a way that its expression becomes essentially the same as the expression in the counterpart normal cell. “Normalization” preferably brings the level of expression to within approximately a 50% difference in expression, more preferably to within approximately a 25%, and even more preferably 10% difference in expression. The required level of closeness in expression will depend on the particular gene, and may be determined as described herein.

“Normalizing gene expression in a diseased lung cell” refers to a means for normalizing the expression of essentially all genes in the diseased lung cell.

“Nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

“Nucleic acid corresponding to a gene” refers to a nucleic acid that may be used for detecting the gene, e.g., a nucleic acid which is capable of hybridizing specifically to the gene.

“Nucleic acid sample derived from RNA” refers to one or more nucleic acid molecule, e.g., RNA or DNA, that was synthesized from the RNA, and includes DNA resulting from methods using PCR, e.g., RT-PCR.

“Panel” as used herein refers to a group of genes and/or their encoded proteins identified via a gene expression profile as being differentially expressed during pathogenesis of lung cells.

“Parenteral administration” and “administered parenterally” means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

A “patient”, “subject” or “host” to be treated by the subject method may mean either a human or non-human animal.

“Peptidomimetic” refers to a compound containing peptide-like structural elements that is capable of mimicking the biological action (s) of a natural parent polypeptide.

“Percent identical” refers to sequence identity between two amino acid sequences or between two nucleotide sequences. Identity may each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules may be referred to as homologous (similar) at that position. Expression as a percentage of homology, similarity, or identity refers to a function of the number of identical or similar amino acids at positions shared by the compared sequences. Various alignment algorithms and/or programs may be used, including, for example, FASTA, BLAST, or ENTREZ. FASTA and BLAST are available as a part of the GCG sequence analysis package (University of Wisconsin, Madison, Wis.), and may be used with, e.g., default settings. ENTREZ is available through the National Center for Biotechnology Information, National Library of Medicine, National Institutes of Health, Bethesda, Md. In one embodiment, the percent identity of two sequences may be determined by the GCG program with a gap weight of 1, e.g., each amino acid gap is weighted as if it were a single amino acid or nucleotide mismatch between the two sequences. Other techniques for alignment include, but are not limited to, those described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Biol. 70: 173-187 (1997). Also, the GAP program using the Needleman and Wunsch alignment method may be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences may be used to search both protein and DNA databases. Databases with individual sequences are described in Methods in Enzymology, ed. Doolittle, supra. Databases include Genbank, EMBL, and DNA Database of Japan (DDBJ).

“Perfectly matched” in reference to a duplex means that the poly- or oligonucleotide strands making up the duplex form a double stranded structure with one other such that every nucleotide in each strand undergoes Watson-Crick basepairing with a nucleotide in the other strand. The term also comprehends the pairing of nucleoside analogs, such as deoxyinosine, nucleosides with 2-aminopurine bases, and the like, that may be employed. A mismatch in a duplex between a target polynucleotide and an oligonucleotide or olynucleotide means that a pair of nucleotides in the duplex fails to undergo Watson-Crick bonding. In reference to a triplex, the term means that the triplex consists of a perfectly matched duplex and a third strand in which every nucleotide undergoes Hoogsteen or reverse Hoogsteen association with a basepair of the perfectly matched duplex.

“Pharmaceutically-acceptable salts” refers to the relatively non-toxic, inorganic and organic acid addition salts of compounds.

“Pharmaceutically-acceptable carrier” refers to a pharmaceutically-acceptable material, composition or vehicle, such as a liquid or solid filler, diluent, excipient, solvent or encapsulating material, involved in carrying or transporting any supplement or composition, or component thereof, from one organ, or portion of the body, to another organ, or portion of the body. Each carrier must be “acceptable” in the sense of being compatible with the other ingredients of the supplement and not injurious to the patient. Any suitable pharmaceutically-acceptable carrier known to or able to be developed by one of skill in the art may be used. Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The “profile” of a cell's biological state refers to the levels of various constituents of a cell that are known to change in response to drug treatments and other perturbations of the cell's biological state. Constituents of a cell include levels of RNA, levels of protein abundances, or protein activity levels.

An expression profile in one cell is “similar” to an expression profile in another cell when the level of expression of the genes in the two profiles are sufficiently similar that the similarity is indicative of a common characteristic, e.g., being one and the same type of cell. Accordingly, the expression profiles of a first cell and a second cell are similar when at least 75% of the genes that are expressed in the first cell are expressed in the second cell at a level that is within a factor of two relative to the first cell.

“Proliferating” and “proliferation” refer to cells undergoing mitosis.

“Prophylactic” or “therapeutic” treatment refers to administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Protein”, “polypeptide” and “peptide” are used interchangeably herein when referring to a gene product, e.g., as may be encoded by a coding sequence. By “gene product” it is meant a molecule that is produced as a result of transcription of a gene. Gene products include RNA molecules transcribed from a gene, as well as proteins translated from such transcripts.

“Recombinant protein”, “heterologous protein” and “exogenous protein” are used interchangeably to refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid.

“Small molecule” refers to a composition, which has a molecular weight of less than about 1000 kDa. Small molecules may be nucleic acids, peptides, polypeptides, peptidomimetics, carbohydrates, lipids or other organic (carbon-containing) or inorganic molecules. As those skilled in the art will appreciate, based on the present description, libraries of chemical and/or biological extensive libraries of chemical and/or biological mixtures, often fungal, bacterial, or algal extracts, may be screened with any of the assays of the invention to identify compounds that modulate a bioactivity.

“Squamous” refers to a cancer whose point of origin was in the squamous epithelial cells found in the skin, the lining of the mouth, the gullet, the airways and fine tubes in the lungs and some other parts of the body. “Squamous cell carcinoma” refers to a cancer of the squamous epithelial cells of the lining of the airways and fine tubes in the lungs.

“Surrogate” refers a biological molecule, e.g., a nucleic acid, peptide, hormone, etc., whose presence, concentration, or level of activity may be detected and correlated with a known condition, such as a disease state.

“Systemic administration,” “administered systemically,” “peripheral administration” and “administered peripherally” refer to the administration of a subject supplement, composition, therapeutic or other material other than directly into the central nervous system, such that it enters the patient's system and, thus, is subject to metabolism and other like processes, for example, subcutaneous administration.

“Therapeutic agent” or “therapeutic” refers to an agent capable of having a desired biological effect on a host. Chemotherapeutic and genotoxic agents are examples of therapeutic agents that are generally known to be chemical in origin, as opposed to biological, or cause a therapeutic effect by a particular mechanism of action, respectively. Examples of therapeutic agents of biological origin include growth factors, hormones, and cytokines. A variety of therapeutic agents are known in the art and may be identified by their effects. Certain therapeutic agents are capable of regulating red cell proliferation and differentiation. Examples include chemotherapeutic nucleotides, drugs, hormones, non-specific (non-antibody) proteins, oligonucleotides (e.g., antisense oligonucleotides that bind to a target nucleic acid sequence (e.g., mRNA sequence)), peptides, and peptidomimetics.

“Therapeutic effect” refers to a local or systemic effect in animals, particularly mammals, and more particularly humans caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds discovered by the methods of the present invention may be administered in a sufficient amount to produce a reasonable benefit/risk ratio applicable to such treatment.

“Treating” a disease in a subject or “treating” a subject having a disease refers to subjecting the subject to a pharmaceutical treatment, e.g., the administration of a drug, such that at least one symptom of the disease is cured, alleviated, decreased or prevented.

“Variant,” when used in the context of a polynucleotide sequence, may encompass a polynucleotide sequence related to that of gene X or the coding sequence thereof. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one base. The presence of SNPs may be indicative of, for example, a certain population, a disease state, or a propensity for a disease state.

A “variant” of polypeptide X refers to a polypeptide having the amino acid sequence of peptide X in which is altered in one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, LASERGENE software (DNASTAR).

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become known in the art subsequently hereto.

3. Novel Targets of the Invention

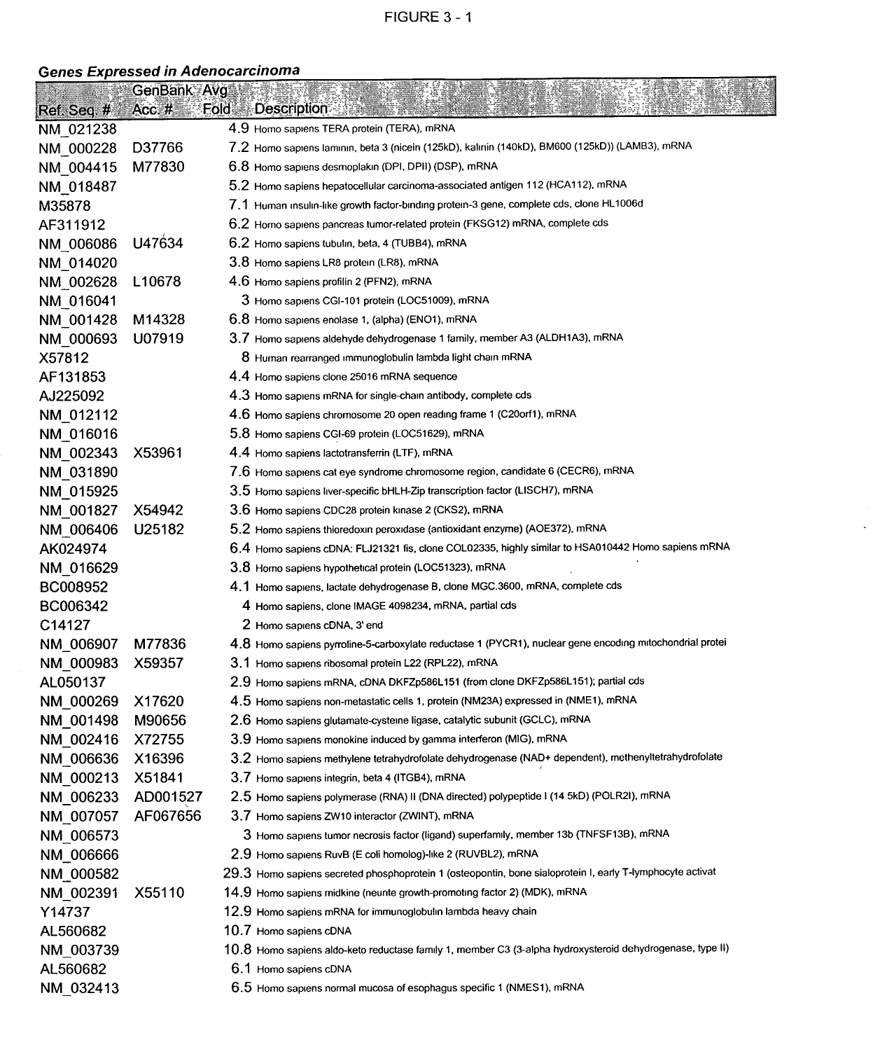

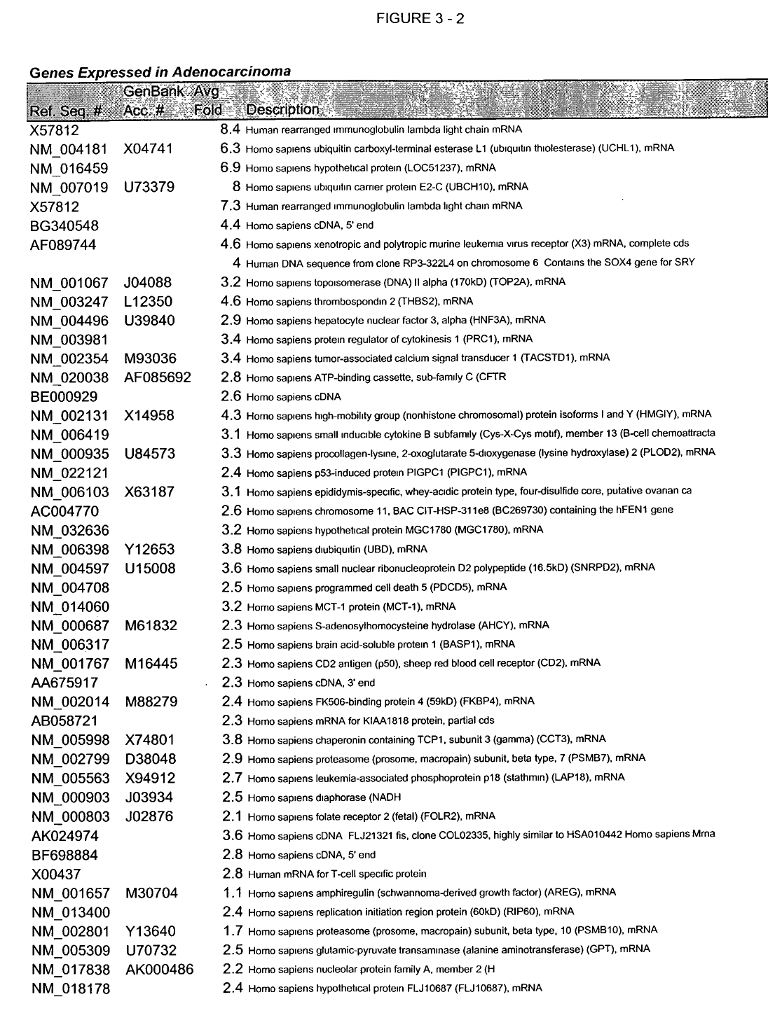

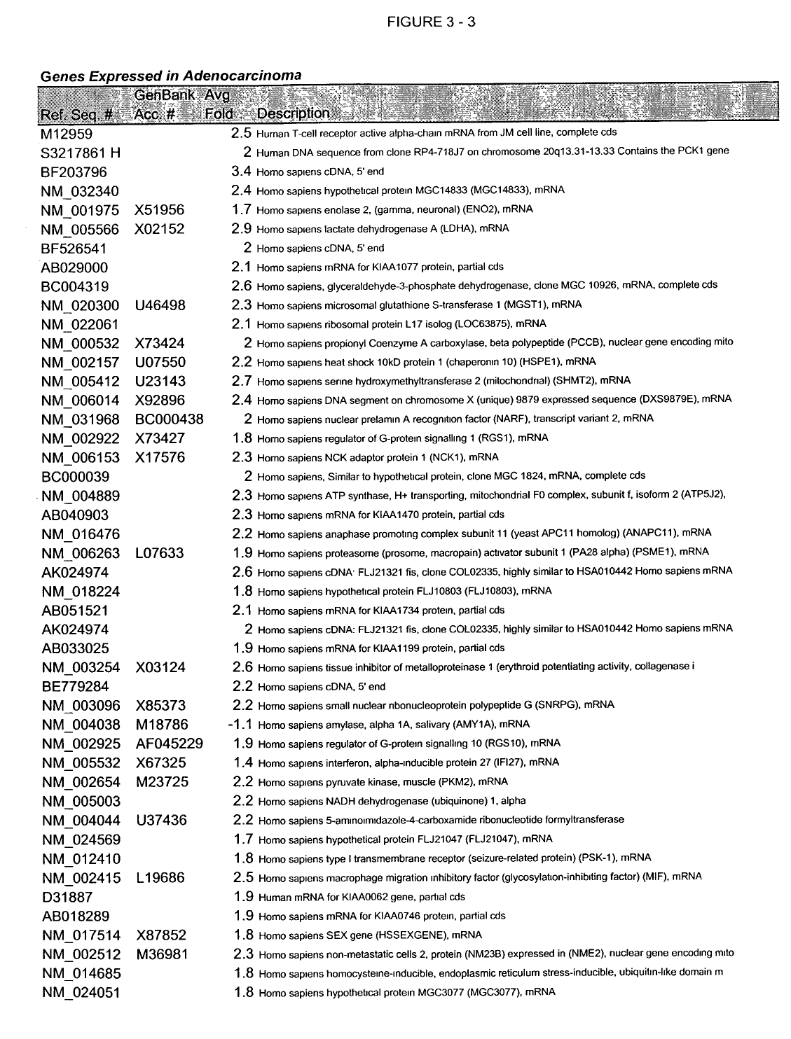

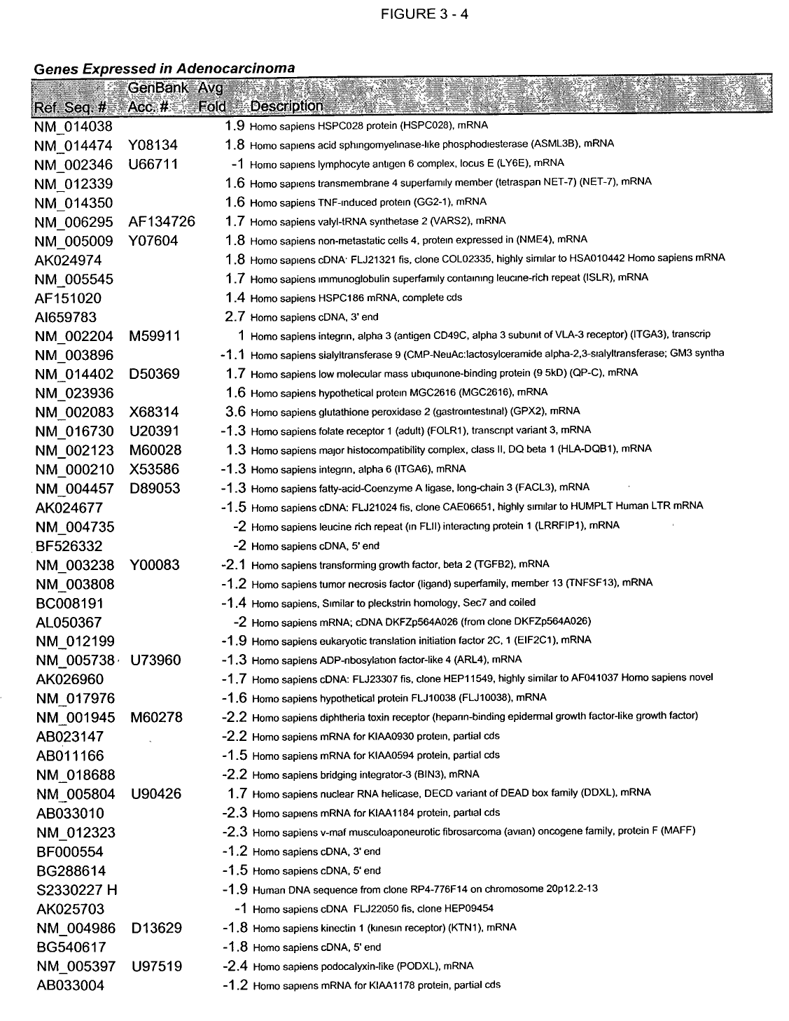

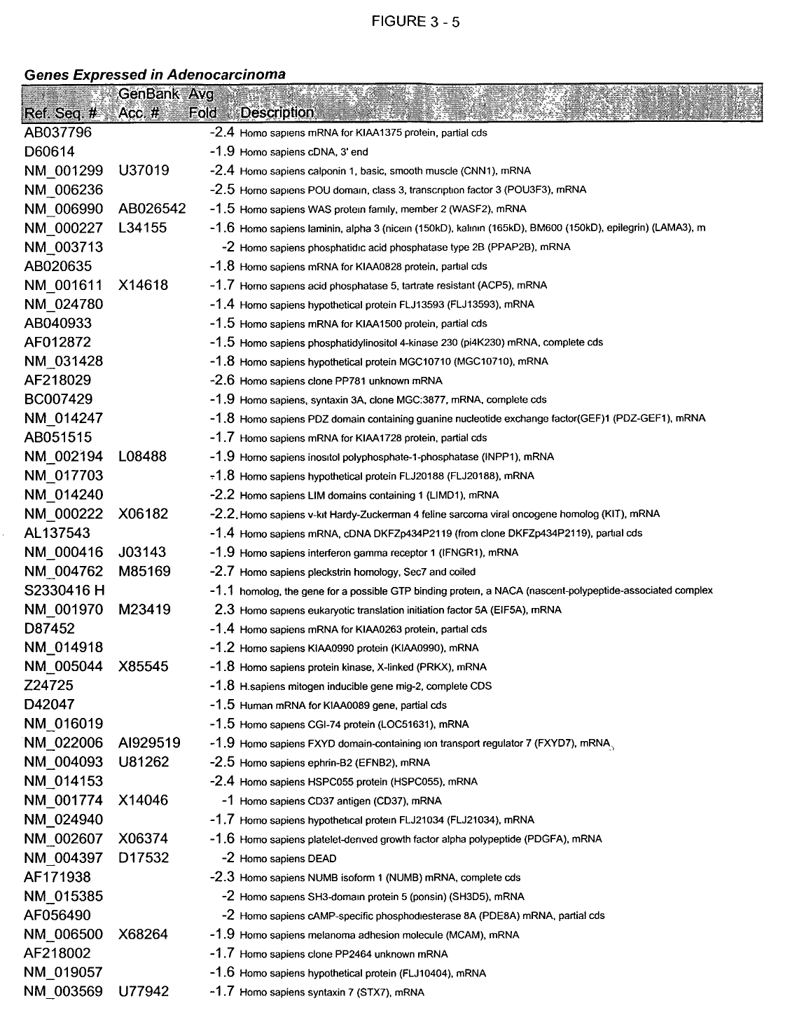

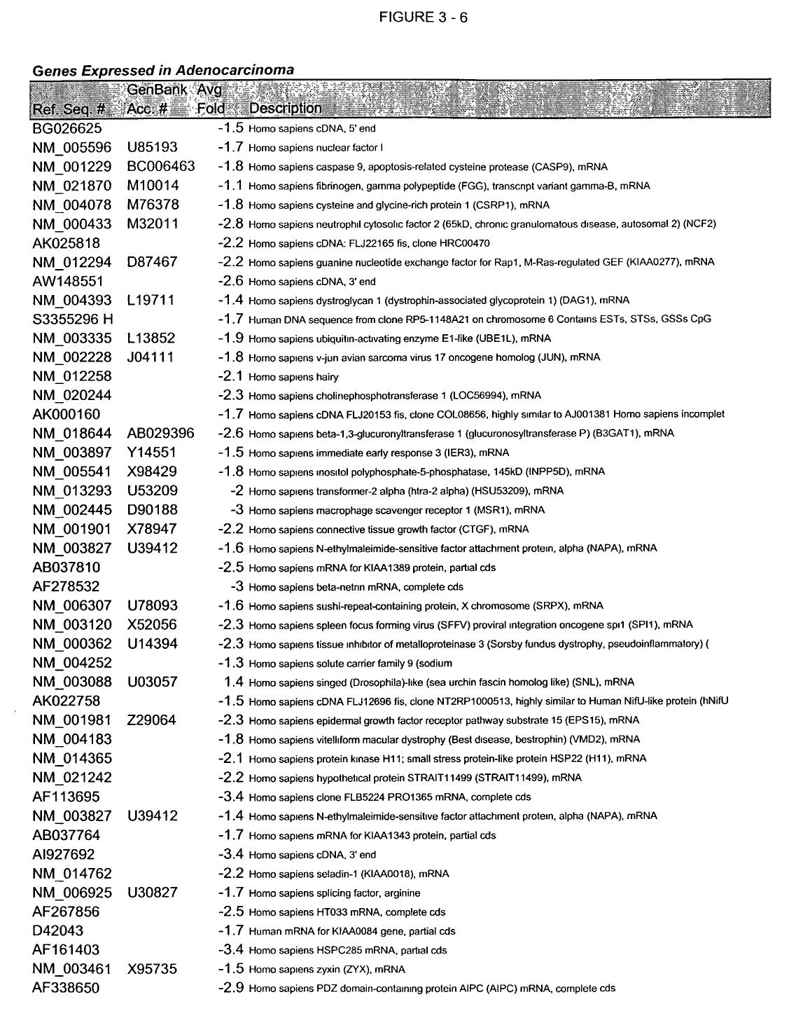

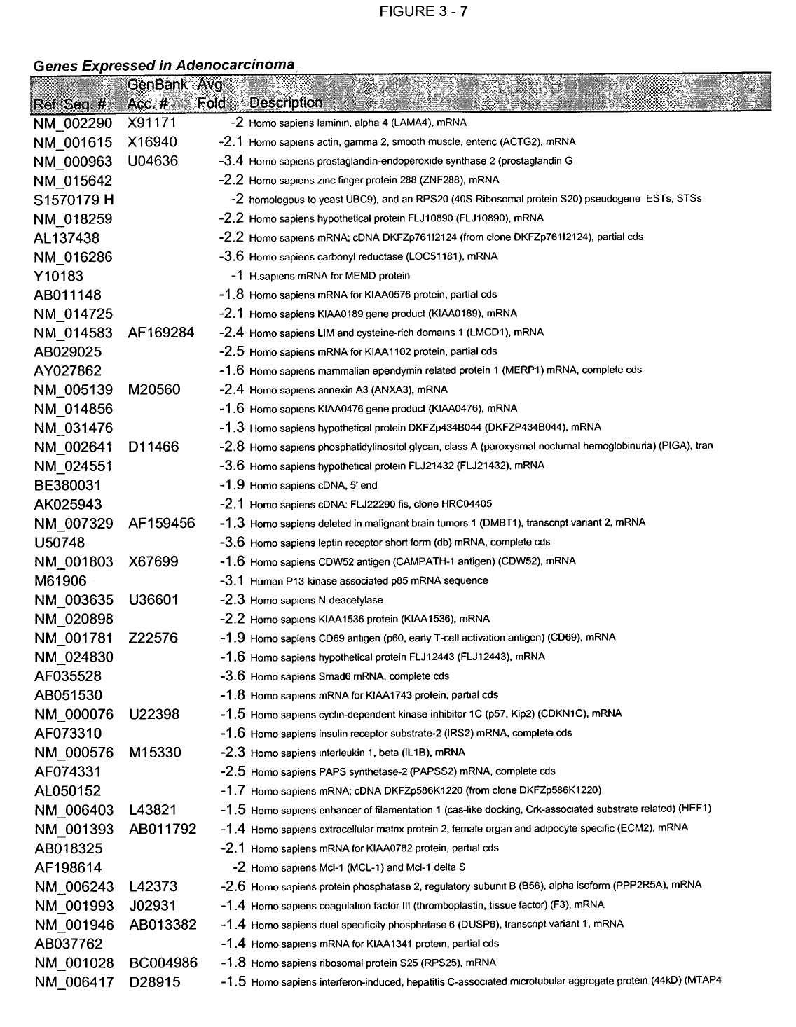

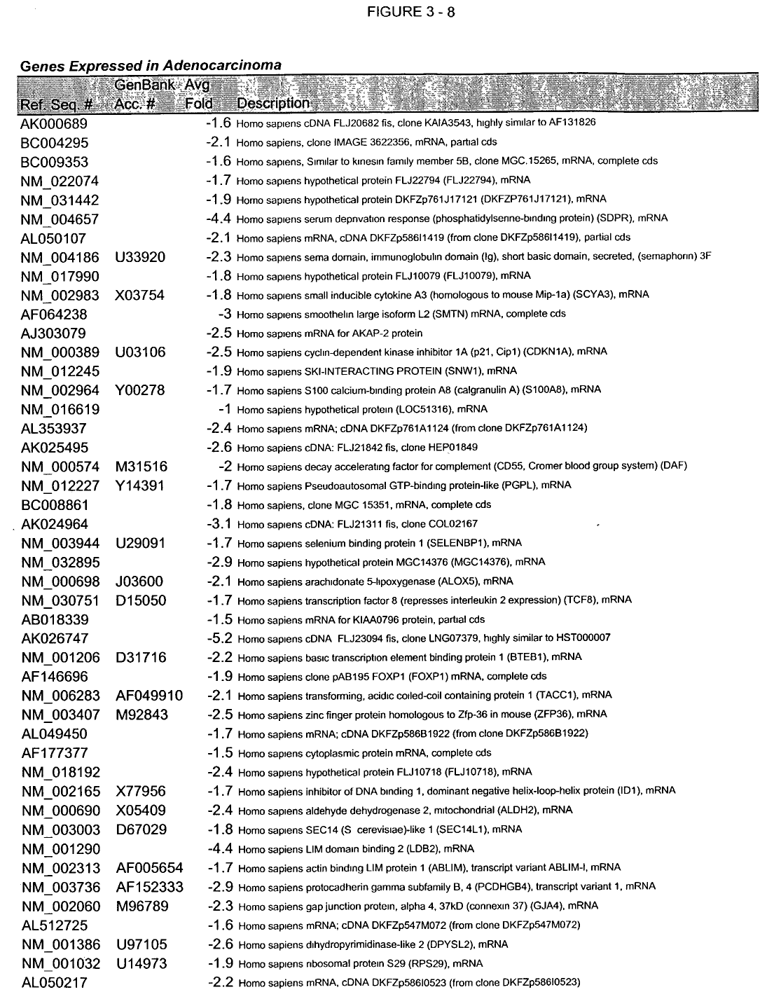

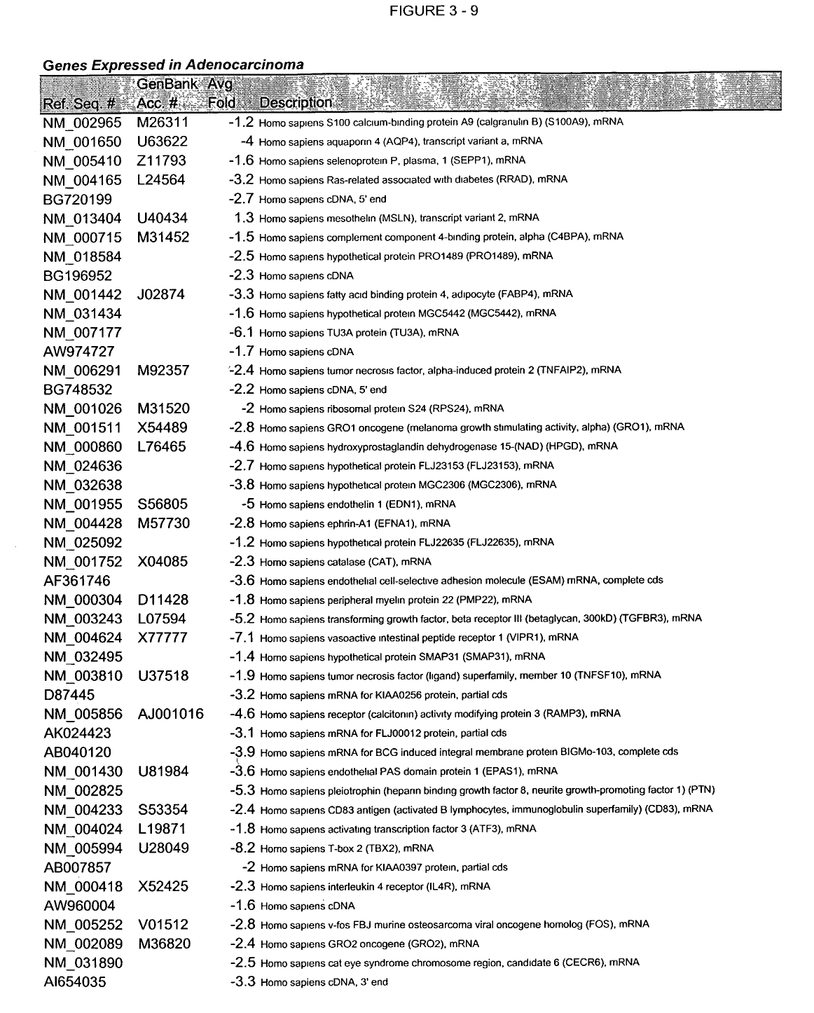

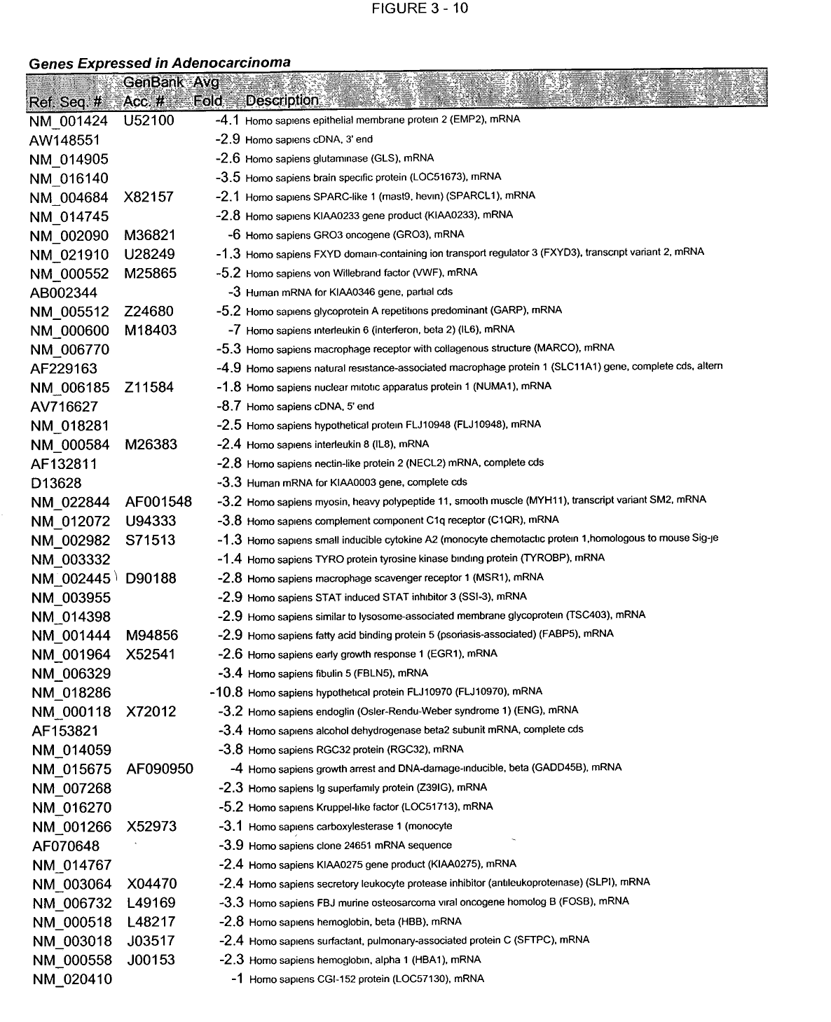

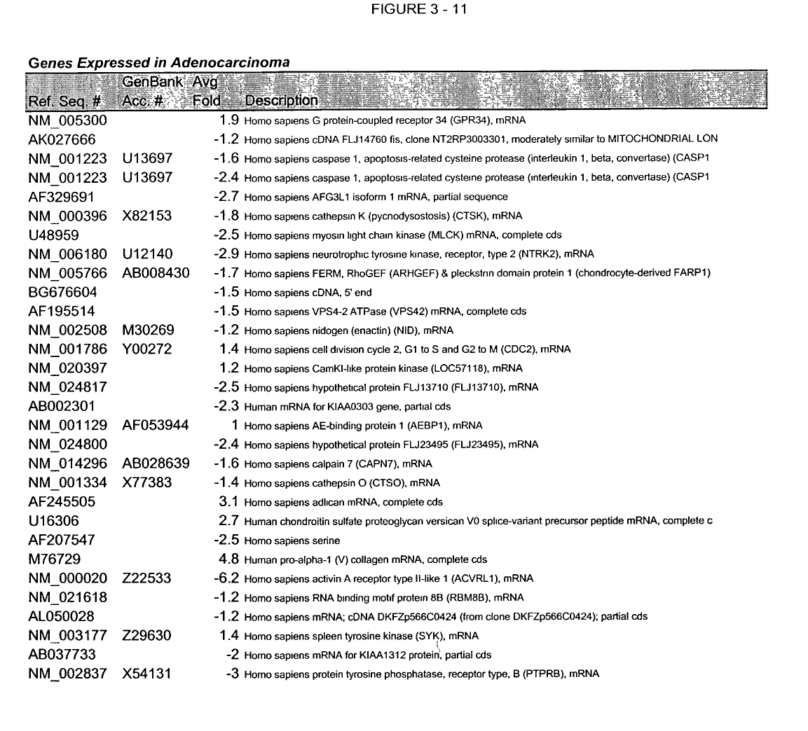

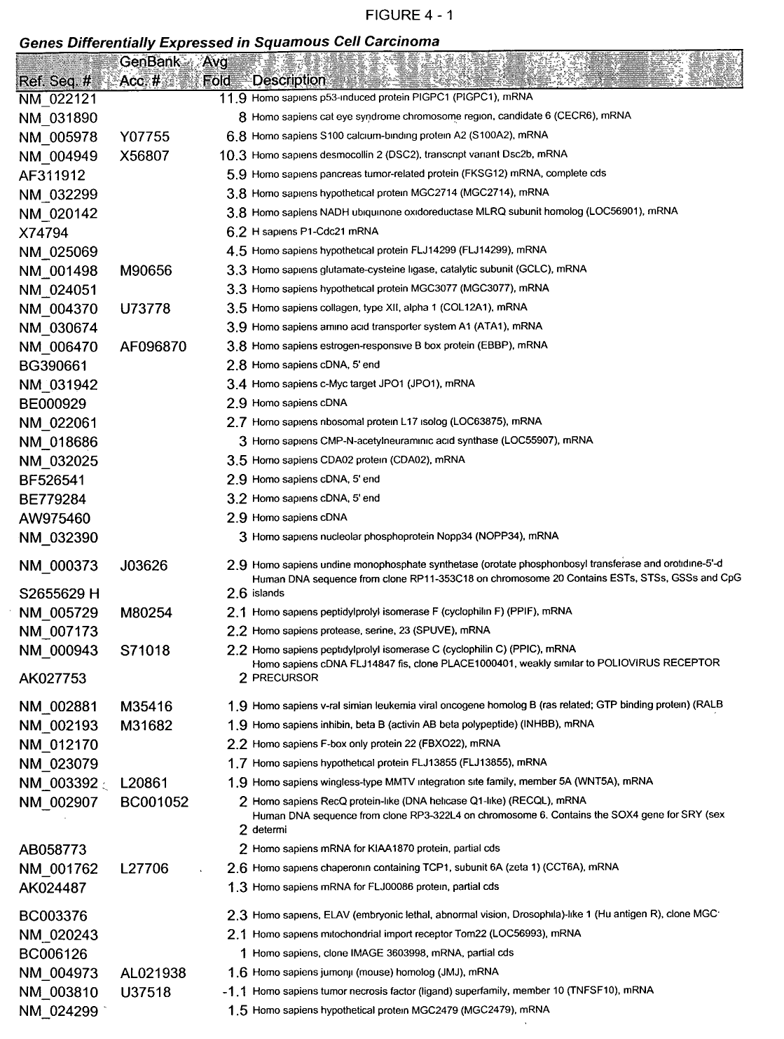

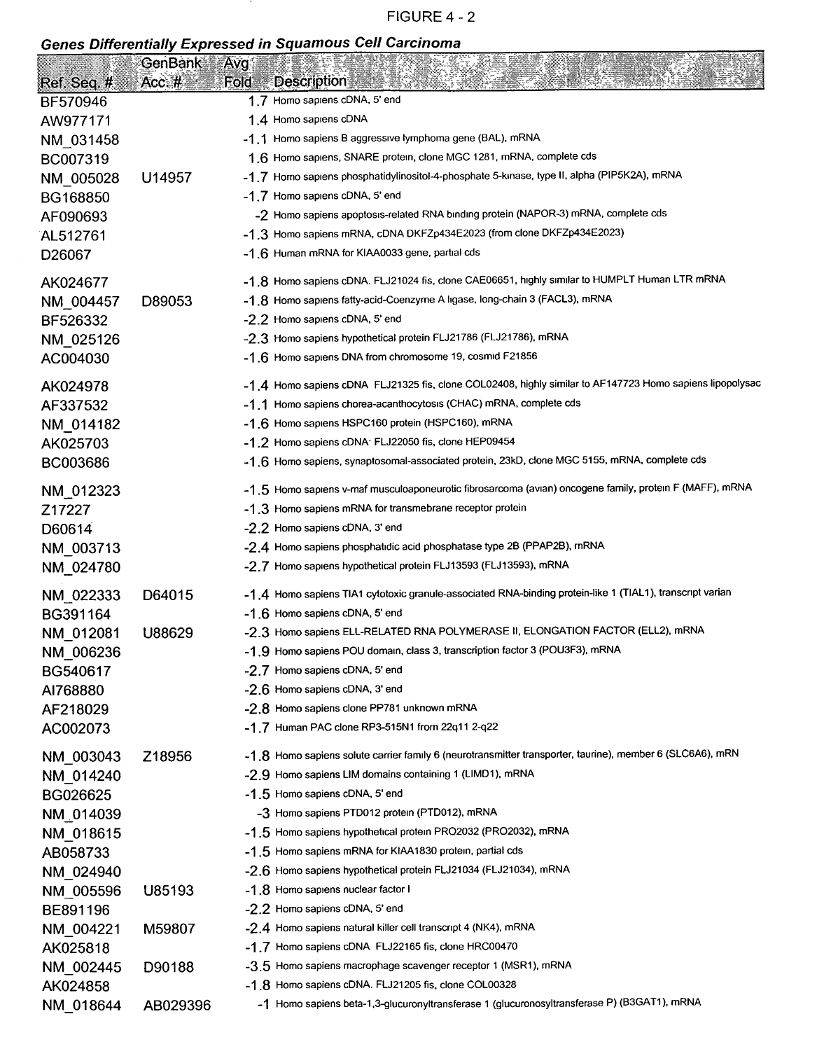

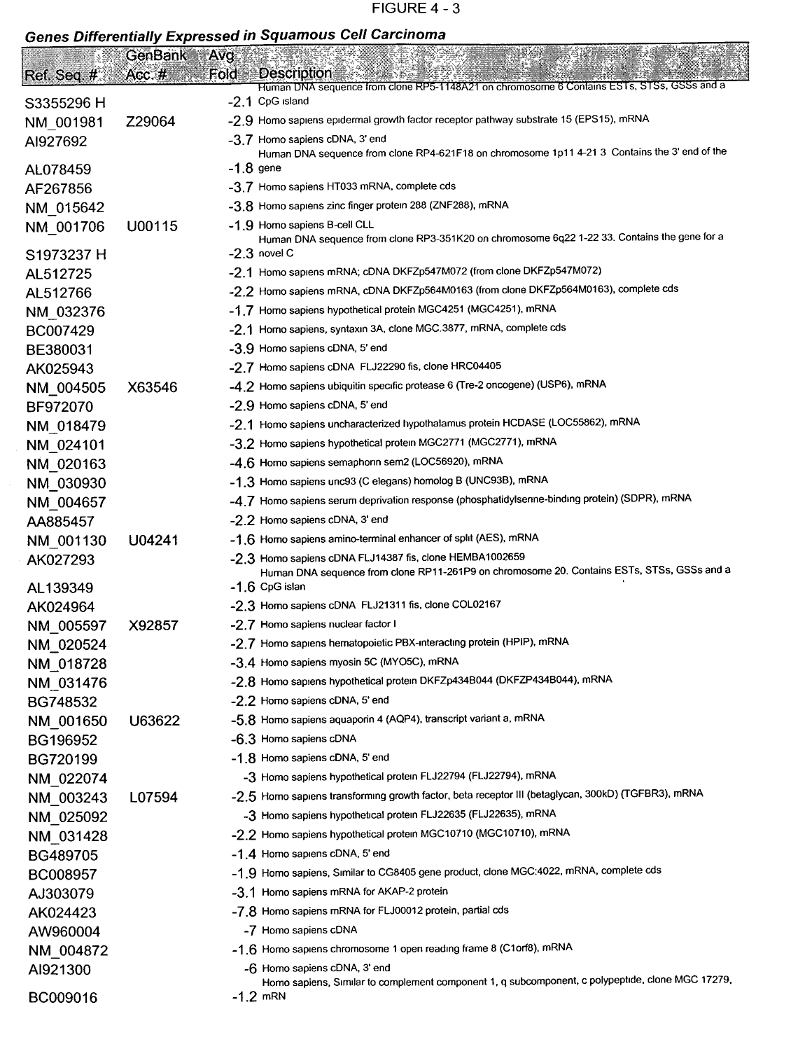

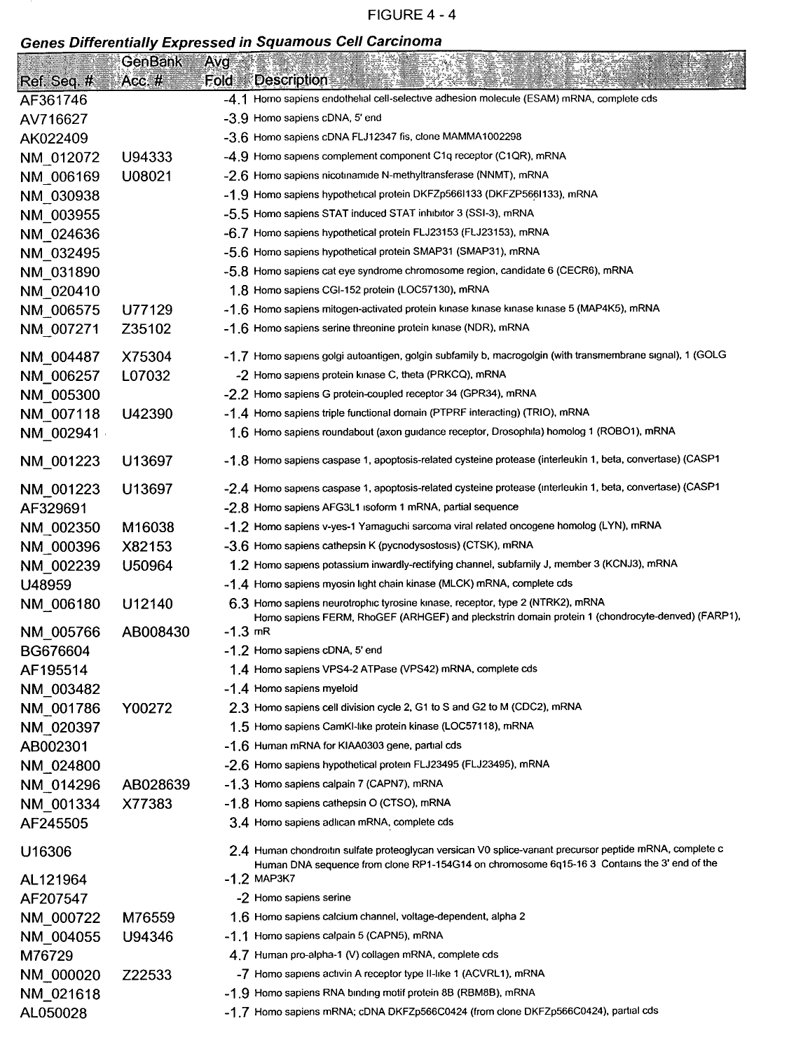

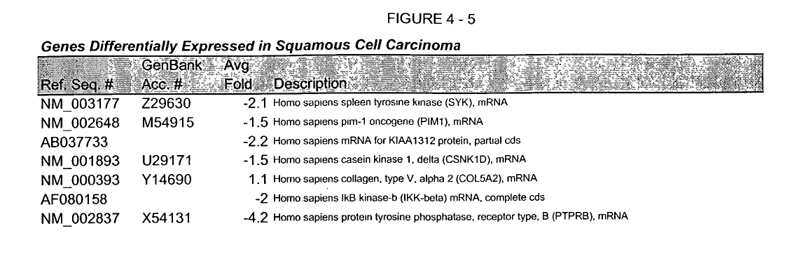

The present invention comprises panels of known genes or gene products that were discovered to exhibit differential expression in lung cells during neoplasia, as identified by gene profiling. In one embodiment, the genes and/or encoded gene products that comprise the panel are selected from the group of genes listed in FIG. 2 that are differentially regulated during pathogenesis of lung cells. In certain embodiments, the genes and/or encoded proteins that comprise the panel are differentially regulated during pathogenesis of lung adenocarcinomas and are selected from the group of genes listed in FIG. 3. In certain embodiments, the genes and/or encoded gene products that comprise the panel are differentially regulated during pathogenesis of lung squamous cell cancers and are selected from the group of genes listed in FIG. 4. As one skilled in the art will appreciate, these genes or their gene products which are differentially regulated in lung tumor cells may be used as targets for diagnostic or therapeutic techniques.

It will be understood by one of skill in the art that multiple entries for a given gene exist in databases, and that the RefSeq numbers and GenBank Accession numbers listed in the FIGURES may represent only one such entry. The database numbers listed in the FIGURES are therefore only one example of the sequence comprising a gene of the panels of the invention. The genes of the panels may comprise the sequences represented by the numbers in the FIGURES, the sequences that comprise other related database entries, sequences with nucleotide substitutions, additions, or deletions, splice variants of the sequences, allelic variants of the sequences, and sequences resulting from the degeneracy of the genetic code, for all of the foregoing and other genes of the invention.

The present invention also relates to TrkB (e.g., RefSeq and GenBank Accession number U12140) and/or its encoded gene product, which was identified by gene expression profiling as being differentially expressed during neoplasia of lung cells. In certain embodiments, the TrkB gene and/or its encoded gene products comprise the “panel” for these methods. The present invention also relates to Aur2 (e.g., RefSeq number NM_{—}003600, GenBank Accession numbers AF011468, AF008551, and BC001280) and/or its encoded gene product, which was also identified by gene expression profiling as being differentially expressed during neoplasia of lung cells. In certain embodiments, the Aur2 gene and/or its encoded gene products comprise the “panel” for these methods.

4. Therapeutics for Early Intervention in Lung Cancer

4.1. Therapeutic Agent Screening

As is well known in the art, lung cancer is the major cause of all cancer-related deaths in Western society. As described above, panels of genes which are differentially regulated during neoplasia of lung cells have been identified, and are provided for use in the present invention as targets in drug design and discovery. In one embodiment of the invention, the cancer is adenocarcinoma and the panel comprises the genes and/or encoded gene products in FIG. 3. In another embodiment of the invention, the cancer is squamous cell carcinoma and the panel comprises the genes and/or encoded gene products in FIG. 4. Individual genes or groups of genes in the panels of the present invention, and/or their encoded gene products, comprise the “targets” for these methods. In some embodiments, candidate therapeutic agents, or “therapeutics” are evaluated for their ability to bind a target protein. The candidate therapeutics may be selected from the following classes of compounds: proteins, peptides, peptidomimetics, or small molecules. In other embodiments, candidate therapeutics are evaluated for their ability to bind a target gene. The candidate therapeutics may be selected from the following classes of compounds: antisense nucleic acids, small molecules, polypeptides, proteins including antibodies, peptidomimetics, or nucleic acid analogs. In some embodiments, the candidate therapeutics are selected from a library of compounds. These libraries may be generated using combinatorial synthetic methods.

The present invention further provides methods for evaluating candidate therapeutic agents of the present invention for their ability to modulate the expression of a target gene by contacting the lung cells of a subject with said candidate therapeutic agents. In certain embodiments, the candidate therapeutic will be evaluated for its ability to normalize the expression levels of a gene or group of genes. Alternatively, candidate therapeutic agents may be evaluated for their ability to inhibit the activity of a protein by contacting the lung cells of a subject with said candidate therapeutic agents. In certain embodiments, a candidate therapeutic may be evaluated for its ability to inhibit the activity of a protein that normally promotes the pathogenesis of lung cancer. These agents would also have utility in asymptomatic individuals at high risk to develop lung cancer.

4.2. Therapeutic Agent Screening Assays

Those skilled in the art will appreciate from the present description that the ability of said candidate therapeutics to bind a target molecule comprising a panel of the present invention may be determined by using any of a variety of suitable assays. For example, in certain embodiments of the present invention, the ability of a candidate therapeutic to bind a target protein or gene may be evaluated by an in vitro assay. In either embodiment, the binding assay may also be an in vivo assay. Assays may be conducted to identify molecules that modulate the expression and or activity of a gene. Alternatively, assays may be conducted to identify molecules that modulate the activity of a protein encoded by a gene.

A person of skill in the art will recognize that in certain screening assays, it will be sufficient to assess the level of expression of a single gene and that in others, the expression of two or more is preferred, whereas still in others, the expression of essentially all the genes involved in lung cell neoplasia is preferably assessed. Likewise, it will be sufficient to assess the activity of a single protein in some screening assays, whereas in others, the activities of multiple proteins may be assessed. Examples of assays that may be used in the present invention include, but are not limited to, competitive binding assay, direct binding assay, two-hybrid assay, cell proliferation assay, kinase assay, phosphatase assay, nuclear hormone translocator assay, and polymerase chain reaction assay. Such assays are well-known to one of skill in the art and, based on the present description, may be adapted to the methods of the present invention with no more than routine experimentation.

All of the above screening methods may be accomplished by using a variety of assay formats. In light of the present disclosure, those not expressly described herein will nevertheless be known and comprehended by one of ordinary skill in the art. The assays may identify agents, e.g., drugs, which are either agonists or antagonists of expression of a target gene of interest, or of a protein:protein or protein-substrate interaction of a target of interest, or of the role of target gene products in the pathogenesis of normal or abnormal cellular physiology, proliferation, and/or differentiation and disorders related thereto. Assay formats which approximate such conditions as formation of protein complexes or protein-nucleic acid complexes, enzymatic activity, and even specific signaling pathways, may be generated in many different forms, as those skilled in the art will appreciate based on the present description and include but are not limited to assays based on cell-free systems, e.g., purified proteins or cell lysates, as well as cell-based assays which utilize intact cells.

As those skilled in the art will understand, based on the present description, binding assays may be used to detect agents which, by disrupting the binding of protein-protein interactions or protein-nucleic acid interactions, or the subsequent binding of such a complex or individual protein or nucleic acid to a substrate, may inhibit signaling or other effects resulting from the given interaction. For example, if one polypeptide binds to another polypeptide, drugs may be developed which modulate the activity of the first polypeptide by modulating its binding to the second polypeptide (referred to herein as a “binding partner” or “binding partner”). Cell-free assays may be used to identify compounds which are capable of interacting with a polypeptide or binding partner, to thereby modify the activity of the polypeptide or binding partner. Such a compound may, e.g., modify the structure of the polypeptide or binding partner and thereby effect its activity. Cell-free assays may also be used to identify compounds which modulate the interaction between a polypeptide and a binding partner. In a preferred embodiment, cell-free assays for identifying such compounds consist essentially in a reaction mixture containing a polypeptide and a test compound or a library of test compounds in the presence or absence of a binding partner. A test compound may be, e.g., a derivative of a binding partner, e.g., a biologically inactive peptide, or a small molecule. Agents to be tested for their ability to act as interaction inhibitors may be produced, for example, by bacteria, yeast or other organisms (e.g., natural products), produced chemically (e.g., small molecules, including peptidomimetics), or produced recombinantly. In a preferred embodiment, the candidate therapeutic agent is a small organic molecule, e.g., other than a peptide or oligonucleotide, having a molecular weight of less than about 2,000 daltons.

In many candidate screening programs which test libraries of compounds and natural extracts, high throughput assays are desirable in order to maximize the number of compounds surveyed in a given period of time. Assays of the present invention which are performed in cell-free systems, such as may be derived with purified or semi-purified proteins or with lysates, are often preferred as “primary” screens in that they may be generated to permit rapid development and often easy detection of an alteration in a molecular target which is mediated by a test compound. Moreover, the effects of cellular toxicity and/or bioavailability of the test compound may be generally ignored in the in vitro system, the assay instead being focused primarily on the effect of the drug on the molecular target as may be manifest in an alteration of binding affinity with other proteins or changes in enzymatic properties of the molecular target. Accordingly, potential modifiers, e.g., activators or inhibitors of protein-substrate, protein-protein interactions or nucleic acid-protein interactions of interest may be detected in a cell-free assay generated by constitution of function interactions of interest in a cell lysate. In an alternate format, the assay may be derived as a reconstituted protein mixture which, as described below, offers a number of benefits over lysate-based assays.

In one aspect, the present invention provides assays that may be used to screen for agents which modulate protein-protein interactions, nucleic acid-protein interactions, or protein-substrate interactions. For instance, the screening assays of the present invention may be designed to detect agents which disrupt binding of protein-protein interaction binding moieties. In other embodiments, the subject assays will identify inhibitors of the enzymatic activity of a protein or protein-protein interaction complex. In a preferred embodiment, the compound is a mechanism based inhibitor which chemically alters one member of a protein-protein interaction or one chemical group of a protein and which is a specific inhibitor of that member, e.g., has an inhibition constant 10-fold, 100-fold, or more preferably, 1000-fold different compared to homologous proteins.

In one embodiment of the present invention, assays are provided which detect inhibitory agents on the basis of their ability to interfere with binding of components of a given protein-substrate, protein-protein, or nucleic acid-protein interaction. In an exemplary binding assay, the compound of interest is contacted with a mixture generated from protein-protein interaction component polypeptides. Detection and quantification of expected activity from a given protein-protein interaction provides a means for determining the compound's efficacy at inhibiting (or potentiating) complex formation between the two polypeptides. The efficacy of the compound may be assessed by generating dose response curves from data obtained using various concentrations of the test compound. Moreover, a control assay may also be performed to provide a baseline for comparison. In the control assay, the formation of complexes is quantitated in the absence of the test compound.

Complex formation between component polypeptides, polypeptides and genes, or between a component polypeptide and a substrate may be detected by a variety of techniques, many of which are effectively described above. For instance, modulation in the formation of complexes may be quantitated using, for example, detectably labeled proteins (e.g., radiolabeled, fluorescently labeled, or enzymatically labeled), by immunoassay, or by chromatographic detection.

Accordingly, one exemplary screening assay of the present invention includes the steps of contacting a polypeptide or functional fragment thereof or a binding partner with a test compound or library of test compounds and detecting the formation of complexes. For detection purposes, for example, the molecule may be labeled with a specific marker and the test compound or library of test compounds labeled with a different marker. Interaction of a test compound with a polypeptide or fragment thereof or binding partner may then be detected by determining the level of the two labels after an incubation step and a washing step. The presence of two labels after the washing step is indicative of an interaction.

An interaction between molecules may also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds may be immobilized on a sensor surface, e.g., which forms one wall of a micro-flow cell. A solution containing the polypeptide, functional fragment thereof, polypeptide analog or binding partner is then flown continuously over the sensor surface. A change in the resonance angle as shown on a signal recording, indicates that an interaction has occurred. This technique is further described, e.g., in BIAtechnology Handbook by Pharmacia.

Another exemplary assay of the present invention includes the steps of (a) forming a reaction mixture including: (i) a polypeptide, (ii) a binding partner, and (iii) a test compound; and (b) detecting interaction of the polypeptide and the binding partner. The polypeptide and binding partner may be produced recombinantly, purified from a source, e.g., plasma, or chemically synthesized, as described herein. A statistically significant change (potentiation or inhibition) in the interaction of the polypeptide and binding partner in the presence of the test compound, relative to the interaction in the absence of the test compound, indicates a potential agonist (mimetic or potentiator) or antagonist (inhibitor) of polypeptide bioactivity for the test compound. The compounds of this assay may be contacted simultaneously. Alternatively, a polypeptide may first be contacted with a test compound for an suitable amount of time, following which the binding partner is added to the reaction mixture. The efficacy of the compound may be assessed by generating dose response curves from data obtained using various concentrations of the test compound. Moreover, a control assay may also be performed to provide a baseline for comparison. In the control assay, isolated and purified polypeptide or binding partner is added to a composition containing the binding partner or polypeptide, and the formation of a complex is quantitated in the absence of the test compound.

Complex formation between a polypeptide and a binding partner may be detected by a variety of techniques. Modulation of the formation of complexes may be quantitated using, for example, detectably labeled proteins such as radiolabeled, fluorescently labeled, or enzymatically labeled polypeptides or binding partners, by immunoassay, or by chromatographic detection.

In a preferred embodiment, it will be desirable to immobilize either polypeptide or its binding partner to facilitate separation of complexes from uncomplexed forms of one or both of the proteins, as well as to accommodate automation of the assay. Binding of polypeptide to a binding partner, may be accomplished in any vessel suitable for containing the reactants. Examples include microtitre plates, test tubes, and micro-centrifuge tubes. In one embodiment, a fusion protein may be provided which adds a domain that allows the protein to be bound to a matrix. For example, glutathione-S-transferase/polypeptide (GST/polypeptide) fusion proteins may be adsorbed onto glutathione sepharose beads (Sigma Chemical, St. Louis, Mo.) or glutathione derivatized microtitre plates, which are then combined with the binding partner, e.g., an ³⁵S-labeled binding partner, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g., beads placed in scintilant), or in the supernatant after the complexes are subsequently dissociated. Alternatively, the complexes may be dissociated from the matrix, separated by SDS-PAGE (sodium dodecyl sulfate-polyacrylamide gel electrophoresis), and the level of polypeptide or binding partner found in the bead fraction quantitated from the gel using standard electrophoretic techniques such as described in the appended examples.

Other techniques for immobilizing proteins on matrices are also available for use in the subject assays. For instance, either the polypeptide or its cognate binding partner may be immobilized utilizing conjugation of biotin and streptavidin. For instance, biotinylated polypeptide molecules may be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptide may be derivatized to the wells of the plate, and polypeptide trapped in the wells by antibody conjugation. As above, preparations of a binding partner and a test compound are incubated in the polypeptide presenting wells of the plate, and the amount of complex trapped in the well may be quantitated. Exemplary methods for detecting such complexes, in addition to those described above for the GST-immobilized complexes, include immunodetection of complexes using antibodies reactive with the binding partner, or which are reactive with polypeptide and compete with the binding partner; as well as enzyme-linked assays which rely on detecting an enzymatic activity associated with the binding partner, either intrinsic or extrinsic activity. In an instance of the latter, the enzyme may be chemically conjugated or provided as a fusion protein with the binding partner. To illustrate, the binding partner may be chemically cross-linked or genetically fused with horseradish peroxidase, and the amount of polypeptide trapped in the complex may be assessed with a chromogenic substrate of the enzyme, e.g., 3,3′-diamino-benzadine terahydrochloride or 4-chloro-1-napthol. Likewise, a fusion protein comprising the polypeptide and glutathione-S-transferase may be provided, and complex formation quantitated by detecting the GST activity using 1-chloro-2,4-dinitrobenzene (Habig et al (1974) J Biol Chem 249:7130).

For processes that rely on immunodetection for quantitating one of the proteins trapped in the complex, antibodies against the protein, such as anti-polypeptide antibodies, may be used. Alternatively, the protein to be detected in the complex may be “epitope tagged” in the form of a fusion protein which includes, in addition to the polypeptide sequence, a second polypeptide for which antibodies are readily available (e.g., from commercial sources). For instance, the GST fusion proteins described above may also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc., New Haven, Conn.) or the pEZZ-protein A system (Pharmacia, N.J.).

In preferred in vitro embodiments of the present assay, the protein or the set of proteins engaged in a protein-protein, protein-substrate, or protein-nucleic acid interaction comprises a reconstituted protein mixture of at least semi-purified proteins. By semi-purified, it is meant that the proteins utilized in the reconstituted mixture have been previously separated from other cellular or viral proteins. For instance, in contrast to cell lysates, the proteins involved in a protein-substrate, protein-protein or nucleic acid-protein interaction are present in the mixture to at least 50% purity relative to all other proteins in the mixture, and more preferably are present at 90-95% purity. In certain embodiments of the subject method, the reconstituted protein mixture is derived by mixing highly purified proteins such that the reconstituted mixture substantially lacks other proteins (such as of cellular or viral origin) which might interfere with or otherwise alter the ability to measure activity resulting from the given protein-substrate, protein-protein interaction, or nucleic acid-protein interaction.

In one embodiment, the use of reconstituted protein mixtures allows more careful control of the protein-substrate, protein-protein, or nucleic acid-protein interaction conditions. Moreover, the system may be derived to favor discovery of inhibitors of particular intermediate states of the protein-protein interaction. For instance, a reconstituted protein assay may be carried out both in the presence and absence of a candidate agent, thereby allowing detection of an inhibitor of a given protein-substrate, protein-protein, or nucleic acid-protein interaction.

Assaying biological activity resulting from a given protein-substrate, protein-protein or nucleic acid-protein interaction, in the presence and absence of a candidate inhibitor, may be accomplished in any vessel suitable for containing the reactants. Examples include microtitre plates, test tubes, and micro-centrifuge tubes.

In a preferred embodiment, it is desirable to immobilize one of the polypeptides to facilitate separation of complexes from uncomplexed forms of one of the proteins, as well as to accommodate automation of the assay. In an illustrative embodiment, a fusion protein may be provided which adds a domain that permits the protein to be bound to an insoluble matrix. For example, protein-protein interaction component fusion proteins may be adsorbed onto glutathione sepharose beads (Sigma Chemical, St. Louis, Mo.) or glutathione derivatized microtitre plates, which are then combined with a potential interacting protein, e.g., an ³⁵S-labeled polypeptide, and the test compound and incubated under conditions conducive to complex formation e.g., at 4° C. in a buffer of 2 mM Tris-HCl (pH 8), 1 nM EDTA, 0.5% Nonidet P-40, and 100 mM NaCl. Following incubation, the beads are washed to remove any unbound interacting protein, and the matrix bead-bound radiolabel determined directly (e.g., beads placed in scintillant), or in the supernatant after the complexes are dissociated, e.g., when microtitre plate is used. Alternatively, after washing away unbound protein, the complexes may be dissociated from the matrix, separated by SDS-PAGE, and the level of interacting polypeptide found in the matrix-bound fraction quantitated from the gel using standard electrophoretic techniques.

In yet another embodiment, the protein-protein interaction component or potential interacting polypeptide may be used to generate an two-hybrid or interaction trap assay (see also, U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696), for subsequently detecting agents which disrupt binding of the interaction components to one another.

In a particular embodiment, the method comprises the use of chimeric genes which express hybrid proteins. To illustrate, a first hybrid gene comprises the coding sequence for a DNA-binding domain of a transcriptional activator may be fused in frame to the coding sequence for a “bait” protein, e.g., a protein-protein interaction component polypeptide of sufficient length to bind to a potential interacting protein. The second hybrid protein encodes a transcriptional activation domain fused in frame to a gene encoding a “fish” protein, e.g., a potential interacting protein of sufficient length to interact with the protein-protein interaction component polypeptide portion of the bait fusion protein. If the bait and fish proteins are able to interact, e.g., form a protein-protein interaction component complex, they bring into close proximity the two domains of the transcriptional activator. This proximity causes transcription of a reporter gene which is operably linked to a transcriptional regulatory site responsive to the transcriptional activator, and expression of the reporter gene may be detected and used to score for the interaction of the bait and fish proteins.

In accordance with the present invention, the method includes providing a host cell, preferably a yeast cell, e.g., Kluyverei lactis, Schizosaccharomyces pombe, Ustilago maydis, Saccharomyces cerevisiae, Neurospora crassa, Aspergillus niger, Aspergillus nidulans, Pichia pastoris, Candida tropicalis, and Hansenula polymorpha, though most preferably S. cerevisiae or S. pombe. The host cell contains a reporter gene having a binding site for the DNA-binding domain of a transcriptional activator used in the bait protein, such that the reporter gene expresses a detectable gene product when the gene is transcriptionally activated. The first chimeric gene may be present in a chromosome of the host cell, or as part of an expression vector.

The host cell also contains a first chimeric gene which is capable of being expressed in the host cell. The gene encodes a chimeric protein, which comprises (i) a DNA-binding domain that recognizes the responsive element on the reporter gene in the host cell, and (ii) a bait protein, such as a protein-protein interaction component polypeptide sequence.

A second chimeric gene is also provided which is capable of being expressed in the host cell, and encodes the “fish” fusion protein. In one embodiment, both the first and the second chimeric genes are introduced into the host cell in the form of plasmids. Preferably, however, the first chimeric gene is present in a chromosome of the host cell and the second chimeric gene is introduced into the host cell as part of a plasmid.

Preferably, the DNA-binding domain of the first hybrid protein and the transcriptional activation domain of the second hybrid protein are derived from transcriptional activators having separable DNA-binding and transcriptional activation domains. For instance, these separate DNA-binding and transcriptional activation domains are known to be found in the yeast GAL4 protein, and are known to be found in the yeast GCN4 and ADR1 proteins. Many other proteins involved in transcription also have separable binding and transcriptional activation domains which make them useful for the present invention, and include, for example, the LexA and VP16 proteins. It will be understood that other (substantially) transcriptionally-inert DNA-binding domains may be used in the subject constructs; such as domains of ACE1, λcI, lac repressor, jun or fos. In another embodiment, the DNA-binding domain and the transcriptional activation domain may be from different proteins. The use of a LexA DNA binding domain provides certain advantages. For example, in yeast, the LexA moiety contains no activation function and has no known effect on transcription of yeast genes. In addition, use of LexA allows control over the sensitivity of the assay to the level of interaction (see, for example, the Brent et al. PCT publication WO94/10300.

In preferred embodiments, any enzymatic activity associated with the bait or fish proteins is inactivated, e.g., dominant negative or other mutants of a protein-protein interaction component may be used.

Continuing with the illustrated example, the protein-protein interaction component-mediated interaction, if any, between the bait and fish fusion proteins in the host cell, therefore, causes the activation domain to activate transcription of the reporter gene. The method is carried out by introducing the first chimeric gene and the second chimeric gene into the host cell, and subjecting that cell to conditions under which the bait and fish fusion proteins and are expressed in sufficient quantity for the reporter gene to be activated. The formation of a protein-protein interaction component/interacting protein complex results in a detectable signal produced by the expression of the reporter gene. Accordingly, the level of formation of a complex in the presence of a test compound and in the absence of the test compound may be evaluated by detecting the level of expression of the reporter gene in each case. Various reporter constructs may be used in accord with the methods of the invention and include, for example, reporter genes which produce such detectable signals as selected from the group consisting of an enzymatic signal, a fluorescent signal, a phosphorescent signal and drug resistance.

One aspect of the present invention provides reconstituted protein preparations, e.g., combinations of proteins participating in protein-protein interactions.

In still further embodiments of the present assay, the protein-protein interaction of interest is generated in whole cells, taking advantage of cell culture techniques to support the subject assay. For example, as described below, the protein-protein interaction of interest may be constituted in a eukaryotic cell culture system, including mammalian and yeast cells. Advantages to generating the subject assay in an intact cell include the ability to detect inhibitors which are functional in an environment more closely approximating that which therapeutic use of the inhibitor would require, including the ability of the agent to gain entry into the cell. Furthermore, certain of the in vivo embodiments of the assay, such as examples given below, are amenable to high through-put analysis of candidate agents.

The components of the protein-protein interaction of interest may be endogenous to the cell selected to support the assay. Alternatively, some or all of the components may be derived from exogenous sources. For instance, fusion proteins may be introduced into the cell by recombinant techniques (such as through the use of an expression vector), as well as by microinjecting the fusion protein itself or mRNA encoding the fusion protein.

The cell is ultimately manipulated after incubation with a candidate inhibitor in order to facilitate detection of a protein-protein interaction-mediated signaling event (e.g., modulation of a post-translational modification of a protein-protein interaction component substrate, such as phosphorylation, modulation of transcription of a gene in response to cell signaling, etc.). As described above for assays performed in reconstituted protein mixtures or lysate, the effectiveness of a candidate inhibitor may be assessed by measuring direct characteristics of the protein-protein interaction component polypeptide, such as shifts in molecular weight by electrophoretic means or detection in a binding assay. For these embodiments, the cell will typically be lysed at the end of incubation with the candidate agent, and the lysate manipulated in a detection step in much the same manner as might be the reconstituted protein mixture or lysate, e.g., described above.

Indirect measurement of protein-protein interaction may also be accomplished by detecting a biological activity associated with a protein-protein interaction component that is modulated by a protein-protein interaction mediated signaling event. As set out above, the use of fusion proteins comprising a protein-protein interaction component polypeptide and an enzymatic activity are representative embodiments of the subject assay in which the detection means relies on indirect measurement of a protein-protein interaction component polypeptide by quantitating an associated enzymatic activity.

In other embodiments, the biological activity of a nucleic acid-protein, protein-substrate or protein-protein interaction component polypeptide may be assessed by monitoring changes in the phenotype of the targeted cell. For example, the detection means may include a reporter gene construct which includes a transcriptional regulatory element that is dependent in some form on the level of an interaction component or a interaction component substrate. The protein interaction component may be provided as a fusion protein with a domain which binds to a DNA element of the reporter gene construct. The added domain of the fusion protein may be one which, through its DNA-binding ability, increases or decreases transcription of the reporter gene. Whichever the case may be, its presence in the fusion protein renders it responsive to the protein-protein interaction-mediated signaling pathway. Accordingly, the level of expression of the reporter gene will vary with the level of expression of the protein interaction component.

The reporter gene product is a detectable label, such as luciferase, β-lactamase or β-galactosidase, and is produced in the intact cell. The label may be measured in a subsequent lysate of the cell. However, the lysis step is preferably avoided, and providing a step of lysing the cell to measure the label will typically only be employed where detection of the label cannot be accomplished in whole cells.

Moreover, in the whole cell embodiments of the subject assay, the reporter gene construct may provide, upon expression, a selectable marker. A reporter gene includes any gene that expresses a detectable gene product, which may be RNA or protein. Preferred reporter genes are those that are readily detectable. The reporter gene may also be included in the construct in the form of a fusion gene with a gene that includes desired transcriptional regulatory sequences or exhibits other desirable properties. For instance, the product of the reporter gene may be an enzyme which confers resistance to antibiotic or other drug, or an enzyme which complements a deficiency in the host cell (e.g., thymidine kinase or dihydrofolate reductase). To illustrate, the aminoglycoside phosphotransferase encoded by the bacterial transposon gene Tn5 neo may be placed under transcriptional control of a promoter element responsive to the level of a protein-protein interaction component polypeptide present in the cell. Such embodiments of the subject assay are particularly amenable to high throughput analysis in that proliferation of the cell may provide a simple measure of inhibition of an interaction.

Reporter genes further include, but are not limited to CAT (chloramphenicol acetyl transferase) (Alton and Vapnek (1979), Nature 282: 864-869) luciferase, and other enzyme detection systems, such as β-galactosidase, β-lactamase, (G. Zlokarnik, et al. (1998) Science, 279:84-88); firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368).

The amount of transcription from the reporter gene may be measured using any method known to those of skill in the art to be suitable. For example, specific mRNA expression may be detected using Northern blots or specific protein product may be identified by a characteristic stain, western blots or an intrinsic activity.

In preferred embodiments, the product of the reporter gene is detected by an intrinsic activity associated with that product. For instance, the reporter gene may encode a gene product that, by enzymatic activity, gives rise to a detection signal based on color, fluorescence, or luminescence.

The amount of expression from the reporter gene is then compared to the amount of expression in either the same cell in the absence of the test compound or it may be compared with the amount of transcription in a substantially identical cell that lacks a component of the protein-protein interaction of interest.

5. Therapeutic Agent Efficacy and Optimization

The present invention provides methods for determining the efficacy of a candidate therapeutic as a drug for lung cancer. In one embodiment, methods for determining efficacy may comprise the steps of a) contacting a candidate therapeutic to a lung tumor cell of a subject; and b) determining the ability of said candidate therapeutic to inhibit pathogenesis of the cell. In another embodiment, methods for determining efficacy may comprise the steps of a) contacting a candidate therapeutic to a lung tumor cell of a subject; and b) determining the ability of said candidate therapeutic to normalize the expression profile of said cell.

Additionally, candidate therapeutics may be screened for efficacy by comparing the expression level of one or more genes associated with lung cell neoplasia after incubating a cell of a subject having lung cancer or similar cell with the test compound. In an even more preferred embodiment, the expression level of the genes-is determined using microarrays, and by comparing the gene expression profile of a cell in response to the test compound with the gene expression profile of a normal cell corresponding to a cell of a subject having lung cancer (a “reference profile”). Optionally the expression profile is also compared to that of a cell from a subject having lung cancer. The comparisons are preferably done by introducing the gene expression profile data of the cell treated with the drug into a computer system comprising reference gene expression profiles which are stored in a computer readable form, using suitable algorithms. Test compounds will be screened for those which alter the level of expression of genes characteristic of the cancer, so as to bring them to a level that is similar to that in a cell of the same type as the diseased cell. Such compounds, i.e., compounds which are capable of normalizing the expression of essentially all genes characteristic of a certain lung cancer, are candidate therapeutics.

The efficacy of the compounds may then be tested in additional in vitro and in vivo assays and in tumor xenograft studies. A test compound may be administered to a test animal and inhibition of tumor growth monitored. Expression of one or more genes characteristic of lung cancer may also be measured before and after administration of the test compound to the animal. A normalization of the expression of one or more of these genes is indicative of the efficiency of the compound for treating lung cancer in the animal.

In another embodiment of the invention, a drug is developed by rational drug design, i.e., it is designed or identified based on information stored in computer readable form and analyzed by algorithms. More and more databases of expression profiles are currently being established, numerous ones being publicly available. By screening such databases for the description of drugs affecting the expression of at least some of the genes characteristic of lung cancer in a manner similar to the change in gene expression profile from a diseased lung cell to that of a normal cell corresponding to the diseased lung cell, compounds may be identified which normalize gene expression in a diseased lung cell. Derivatives and analogues of such compounds may then be synthesized to optimize the activity of the compound, and tested and optimized as described above.

Compounds identified by the methods described above are within the scope of the invention. Compositions comprising such compounds, in particular, compositions comprising a pharmaceutically efficient amount of the drug in a pharmaceutically-acceptable carrier are also provided. Certain compositions comprise one or more active compound for treating lung cancer.

The invention also provides methods for designing therapeutics for treating related cancers. Related diseases may in fact have a gene expression profile, which even though not identical to that of lung cancer, will show some homology, so that drugs for treating lung cancer may be used for starting the research of compounds for treating the related disease. A compound for treating lung cancer may be derivatized and tested as further described herein.

6. Pharmaceutical Compositions of Therapeutic Agents

The therapeutic agents identified using the methods provided by the invention may be incorporated into pharmaceutical composition. For example, pharmaceutical compositions may comprise a therapeutic agents and, e.g., a pharmaceutically-acceptable carrier, vehicle, excipient, or diluent. The compounds of the present invention may be administered by any suitable means, depending, for example, on their intended use, as is well known in the art, based on the present description. For example, if compounds of the present invention are to be administered orally, they may be formulated as tablets, capsules, granules, powders or syrups. Alternatively, formulations of the present invention may be administered parenterally as injections (intravenous, intramuscular or subcutaneous), drop infusion preparations or suppositories. For application by the ophthalmic mucous membrane route, compounds of the present invention may be formulated as eyedrops or eye ointments. These formulations may be prepared by conventional means, and, if desired, the compounds may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

Subject compounds may be suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of agent that may be combined with a carrier material to produce a single dose vary depending upon the subject being treated, and the particular mode of administration.

Methods of preparing these formulations can include the step of bringing into association agents of the present invention with the carrier, vehicle or diluent and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of, e.g., capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a compound thereof as an active ingredient. Compounds of the present invention may also be administered as a bolus, electuary, or paste.

In solid dosage forms for oral administration (capsules, tablets, pills, dragees, powders, granules and the like), the therapeutic agent is mixed with one or more pharmaceutically-acceptable carriers, such as, e.g., sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the supplement or components thereof moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulation art.

Liquid dosage forms for oral administration include pharmaceutically-acceptable emulsions, microemulsions, solutions, suspensions, syrups and elixirs. In addition to the compound, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to compounds, may contain suspending agents as, for example, ethoxylated isostearyl alcohols, polyoxyethylencoordinatione sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a therapeutic agent of the present invention with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent. Formulations which are suitable for vaginal administration also include pessaries, tampons, creams, gels, pastes, foams or spray formulations containing such carriers as are known in the art to be suitable.

Dosage forms for transdermal administration of a supplement or component includes powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required. For transdermal administration of transition metal complexes, the complexes may include lipophilic and hydrophilic groups to achieve the desired water solubility and transport properties.

The ointments, pastes, creams and gels may contain, in addition to a supplement or components thereof, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a supplement or components thereof, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compounds of the present invention may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compound.

Ordinarily, an aqueous aerosol is made by formulating an aqueous solution or suspension of the compound together with conventional pharmaceutically-acceptable carriers and stabilizers. The carriers and stabilizers vary with the requirements of the particular compound, but typically include non-ionic surfactants (Tween®s, Pluronic®s, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this invention suitable for parenteral administration comprise one or more components of a supplement in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

7. Methods of Treating Lung Cancer Using Pharmaceutical Compositions

The pharmaceutical compositions of the present invention may be used in a variety methods for treating lung cancer. In one embodiment, methods for treating a subject having lung cancer may comprise administering a therapeutically-effective amount of a pharmaceutical composition to said subject to modulate the expression of a gene or group of genes selected from the target genes of the invention. In another embodiment, methods for treating a subject that has lung cancer may comprise administering a therapeutically-effective amount of a pharmaceutical composition to said subject to inhibit the activity of a protein encoded by a gene selected from the target genes of the invention. In still another embodiment, methods for treating a subject that has lung cancer may comprise administering a therapeutically-effective amount of a pharmaceutical composition or compositions to said subject to normalize the expression profile of the subject's lung cells. In an alternative embodiment of the present invention, methods of treating a subject having lung cancer comprise administering to said subject a protein encoded by the panels of the present invention whose levels are deficient during lung cell pathogenesis.

The pharmaceutical compositions of the present invention may be used preventatively to treat a subject who has had or who may be at risk of developing lung cancer, e.g., in a cancer chemoprevention regimen.

As those skilled in the art will understand, the dosage of any agent (compound, drug, etc.) of the present invention will vary depending on the symptoms, age and body weight of the patient, the nature and severity of the disorder to be treated or prevented, the route of administration, and the form of the supplement. Any of the subject formulations may be administered in any suitable dose, such as, for example, in a single dose or in divided doses. Dosages for the compounds of the present invention, alone or together with any other compound of the present invention, or in combination with any compound deemed useful for the particular disorder, disease or condition sought to be treated, may be readily determined by techniques known to those of skill in the art, based on the present description, and as taught herein. Also, the present invention provides mixtures of more than one subject compound, as well as other therapeutic agents.

The precise time of administration and amount of any particular compound that will yield the most effective treatment in a given patient will depend upon the activity, pharmacokinetics, and bioavailability of a particular compound, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more relevant indices at predetermined times during a 24-hour period. Treatment, including supplement, amounts, times of administration and formulation, may be optimized according to the results of such monitoring. The patient may be periodically reevaluated to determine the extent of improvement by measuring the same parameters, the first such reevaluation typically occurring at the end of four weeks from the onset of therapy, and subsequent reevaluations occurring every four to eight weeks during therapy and then every three months thereafter. Therapy may continue for several months or even years, with a minimum of one month being a typical length of therapy for humans. Adjustments to the amount(s) of agent administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The combined use of several compounds of the present invention, or alternatively other chemotherapeutic agents, may reduce the required dosage for any individual component because the onset and duration of effect of the different components may be complimentary. In such combined therapy, the different active agents may be delivered together or separately, and simultaneously or at different times within the day.

8. Kits for the Treatment of Lung Cancer

The present invention provides kits for treating lung cancer. For example, a kit may also comprise one or more nucleic acids corresponding to one or more genes characteristic of lung cancer, e.g., for use in treating a patient having that cancer. The nucleic acids may be included in a plasmid or a vector, e.g., a viral vector. Other kits comprise a polypeptide encoded by a gene characteristic of lung cancer or an antibody to a polypeptide. Yet other kits comprise compounds identified herein as agonists or antagonists of genes characteristic of lung cancer. The compositions may be pharmaceutical compositions comprising a pharmaceutically-acceptable excipient.

For example, a kit may also comprise one or more nucleic acids corresponding to TrkB, e.g., for use in treating a patient having that cancer. The nucleic acids may be included in a plasmid or a vector, e.g., a viral vector. Other kits comprise a polypeptide encoded by TrkB or an antibody to a polypeptide. Yet other kits comprise compounds identified herein as agonists or antagonists of TrkB. In another example, a kit may also comprise one or more nucleic acids corresponding to Aur2, e.g., for use in treating a patient having that cancer. The nucleic acids may be included in a plasmid or a vector, e.g., a viral vector. Other kits comprise a polypeptide encoded by Aur2 or an antibody to a polypeptide. Yet other kits comprise compounds identified herein as agonists or antagonists of Aur2.

Kit components may be packaged for either manual or partially or wholly automated practice of the foregoing methods. In other embodiments involving kits, this invention provides a kit including compositions of the present invention. Any of the above-described kits may optionally include instructions for their use. Such kits may have a variety of uses, including, for example, imaging, diagnosis, and therapy.

9. Compositions Comprising Probes Derived from Targets of the Invention

The present invention provides compositions comprised of probes derived from the sequences of the genes or proteins encoded by them comprising the panels of the present invention. These compositions may be used in diagnostic applications as discussed herein. Preferred compositions for use according to the invention include one or more probes of genes whose expression is characteristic of lung cancer selected from the panels in FIG. 2. In certain embodiments, the probes of the composition are derived from nucleic acid sequences selected from the target genes whose expression is characteristic of adenocarcinoma listed in FIG. 3. In still other embodiments, the probes of the composition are derived from the nucleic acid sequences selected from target genes whose expression is characteristic of squamous cell carcinoma listed in FIG. 4. The composition may comprise probes corresponding to at least 10, preferably at least 20, at least 50, at least 100 or at least 1000 genes involved in neoplasia. The composition may comprise probes corresponding to each gene listed in FIG. 2, 3 or 4, or subsets of those genes in FIG. 2, 3, or 4 which are up-regulated or down-regulated during neoplasia of lung cells. In certain embodiments, the composition comprises a probe derived from the nucleic acid sequence of TrkB. In other embodiments, the composition comprises a probe derived from the nucleic acid sequence of Aur2.

In one embodiment of the present invention, the composition is a microarray. There may be one or more than one probe corresponding to each gene on a microarray. For example, a microarray may contain from 2 to 20 probes corresponding to one gene and preferably about 5 to 10. The probes may correspond to the full length RNA sequence or complement thereof of genes involved in pathogenesis of lung cells, or they may correspond to a portion thereof, which portion is of sufficient length for permitting specific hybridization. Such probes may comprise from about 50 nucleotides to about 100, 200, 500, or 1000 nucleotides or more than 1000 nucleotides. As further described herein, microarrays may contain oligonucleotide probes, consisting of about 10 to 50 nucleotides, preferably about 15 to 30 nucleotides and even more preferably 20-25 nucleotides. The probes are preferably single stranded. The probe will have sufficient complementarity to its target to provide for the desired level of sequence specific hybridization (see below).

Typically, the arrays used in the present invention will have a site density of greater than 100 different probes per cm², although any suitable site density is included in the present invention. Preferably, the arrays will have a site density of greater than 500/cm², more preferably greater than about 1000/cm², and most preferably, greater than about 10,000/cm². Preferably, the arrays will have more than 100 different probes on a single substrate, more preferably greater than about 1000 different probes still more preferably, greater than about 10,000 different probes and most preferably, greater than 100,000 different probes on a single substrate.

Microarrays maybe prepared by methods known in the art, as described below, or they may be custom made by companies, e.g., Affymetrix.

Generally, two types of microarrays maybe used. These two types are referred to as “synthesis” and “delivery.” In the synthesis type, a microarray is prepared in a step-wise fashion by the in situ synthesis of nucleic acids from nucleotides. With each round of synthesis, nucleotides are added to growing chains until the desired length is achieved. In the delivery type of microarray, pre-prepared nucleic acids are deposited onto known locations using a variety of delivery technologies. Numerous articles describe the different microarray technologies, e.g., Shena et al. (1998) Tibtech 16: 301; Duggan et al. (1999) Nat. Genet. 21: 10; Bowtell et al. (1999) Nat. Genet. 21: 25.

One novel synthesis technology is that developed by Affymetrix, which combines photolithography technology with DNA synthetic chemistry to enable high density oligonucleotide microarray manufacture. Such chips contain up to 400,000 groups of 2 oligonucleotides in an area of about 1.6 cm². Oligonucleotides are anchored at the 3′ end thereby maximizing the availability of single-stranded nucleic acid for hybridization. Generally such chips, referred to as “GeneChips®” contain several oligonucleotides of a particular gene, e.g., between 15-20, such as 16 oligonucleotides. Custom-made microarrays are commercially available, e.g., a microarray for genes involved in lung cancer, and may be purchased from vendors such as Affymetrix.

Microarrays may also be prepared by mechanical microspotting, e.g., those commercialized at Synteni (Fremont, Calif.). According to these methods, small quantities of nucleic acids are printed onto solid surfaces. Microspotted arrays prepared at Synteni contain as many as 10,000 groups of cDNA in an area of about 3.6 cm².

A third group of microarray technologies consist in the “drop-on-demand” delivery approaches, the most advanced of which are the ink-jetting technologies, which utilize piezoelectric and other forms of propulsion to transfer nucleic acids from miniature nozzles to solid surfaces. Inkjet technologies is developed at several centers including Incyte Pharmaceuticals (Palo Alto, Calif.) and Protogene (Palo Alto, Calif.). This technology results in a density of 10,000 spots per cm². See also, Hughes et al. (2001) Nat. Biotechn. 19:342.

Arrays preferably include control and reference nucleic acids. Control nucleic acids are nucleic acids which serve to indicate that the hybridization was effective. For example, all Affymetrix expression arrays contain sets of probes for several prokaryotic genes, e.g., bioB, bioC and bioD from biotin synthesis of E. coli and cre from P1 bacteriophage. Hybridization to these arrays is conducted in the presence of a mixture of these genes or portions thereof, such as the mix provided by Affymetrix to that effect (Part Number 900299), to thereby confirm that the hybridization was effective. Control nucleic acids included with the target nucleic acids may also be mRNA synthesized from cDNA clones by in vitro transcription. Other control genes that may be included in arrays are polyA controls, such as dap, lys, phe, thr, and trp (which are included on Affymetrix GeneChips®)

Reference nucleic acids allow the normalization of results from one experiment to another, and to compare multiple experiments on a quantitative level. Exemplary reference nucleic acids include housekeeping genes of known expression levels, e.g., GAPDH, hexokinase and actin.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases.

Arrays may also contain probes that hybridize to more than one allele of a gene. For example the array may contain one probe that recognizes allele 1 and another probe that recognizes allele 2 of a particular gene.

Microarrays may be prepared in any manner, such as for example, an array of oligonucleotides may be synthesized on a solid support. Exemplary solid supports include glass, plastics, polymers, metals, metalloids, ceramics, organics, etc. Using chip masking technologies and photoprotective chemistry it is possible to generate ordered arrays of nucleic acid probes. These arrays, which are known, e.g., as “DNA chips,” or as very large scale immobilized polymer arrays (“VLSIPS™” arrays) may include millions of defined probe regions on a substrate having an area of about 1 cm² to several cm², thereby incorporating sets of from a few to millions of probes (see, e.g., U.S. Pat. No. 5,631,734).

The construction of solid phase nucleic acid arrays to detect target nucleic acids is well described in the literature. See, Fodor et al. (1991) Science, 251: 767-777; Sheldon et al. (1993) Clinical Chemistry 39(4): 718-719; Kozal et al. (1996) Nature Medicine 2(7): 753-759 and Hubbell U.S. Pat. No. 5,571,639; Pinkel et al. PCT/US95/16155 (WO 96/17958); U.S. Pat. Nos. 5,677,195; 5,624,711; 5,599,695; 5,451,683; 5,424,186; 5,412,087; 5,384,261; 5,252,743 and 5,143,854; PCT WO 92/10092 and 93/09668; and PCT WO 97/10365. In brief, a combinatorial strategy allows for the synthesis of arrays containing a large number of probes using a minimal number of synthetic steps. For instance, it is possible to synthesize and attach all possible DNA 8 mer oligonucleotides (48, or 65,536 possible combinations) using only 32 chemical synthetic steps. In general, VLSIPS™ procedures provide a method of producing 4n different oligonucleotide probes on an array using only 4n synthetic steps (see, e.g., U.S. Pat. Nos. 5,631,734; 5,143,854 and PCTs WO 90/15070; WO 95/11995 and WO 92/10092).

Light-directed combinatorial synthesis of oligonucleotide arrays on a glass surface may be performed with automated phosphoramidite chemistry and chip masking techniques similar to photoresist technologies in the computer chip industry. Typically, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′-photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface.

Algorithms for design of masks to reduce the number of synthesis cycles are described by Hubbel et al., U.S. Pat. Nos. 5,571,639 and 5,593,839. A computer system may be used to select nucleic acid probes on the substrate and design the layout of the array as described, e.g., in U.S. Pat. No. 5,571,639.

Another method for synthesizing high density arrays is described, e.g., in U.S. Pat. No. 6,083,697. This method utilizes a novel chemical amplification process using a catalyst system which is initiated by radiation to assist in the synthesis the polymer sequences. Methods of the present invention include the use of photosensitive compounds which act as catalysts to chemically alter the synthesis intermediates in a manner to promote formation of polymer sequences. Such photosensitive compounds include what are generally referred to as radiation-activated catalysts (RACs), and more specifically photo activated catalysts (PACs). The RACs may by themselves chemically alter the synthesis intermediate or they may activate an autocatalytic compound which chemically alters the synthesis intermediate in a manner to allow the synthesis intermediate to chemically combine with a later added synthesis intermediate or other compound.

Arrays may also be synthesized in a combinatorial fashion by delivering monomers to cells of a support by mechanically constrained flowpaths. See Winkler et al., EP 624,059. Arrays may also be synthesized by spotting monomers reagents on to a support using an ink jet printer. See id. and Pease et al., EP 728,520.

cDNA probes may be prepared according to methods known in the art and further described herein, e.g., reverse-transcription PCR (RT-PCR) of RNA using sequence specific primers. Oligonucleotide probes may be synthesized chemically. Sequences of the genes or cDNA from which probes are made may be obtained, e.g., from GenBank, other public databases or publications.

Nucleic acid probes may be natural nucleic acids, chemically modified nucleic acids, e.g., composed of nucleotide analogs, as long as they have activated hydroxyl groups compatible with the linking chemistry. The protective groups can, themselves, be photolabile. Alternatively, the protective groups may be labile under certain chemical conditions, e.g., acid. In this example, the surface of the solid support may contain a composition that generates acids upon exposure to light. Thus, exposure of a region of the substrate to light generates acids in that region that remove the protective groups in the exposed region. Also, the synthesis method may use 3′ protected 5′-0-phosphoramidite-activated deoxynucleoside. In this case, the oligonucleotide is synthesized in the 5′ to 3′ direction, which results in a free 5′ end.

In one embodiment, oligonucleotides of an array are synthesized using a 96 well automated multiplex oligonucleotide synthesizer (A.M.O.S.) that is capable of making thousands of oligonucleotides (Lashkari et al. (1995) PNAS 93: 7912) may be used.

It will be appreciated that oligonucleotide design is influenced by the intended application. For example, it may be desirable to have similar melting temperatures for all of the probes. Accordingly, the length of the probes are adjusted so that the melting temperatures for all of the probes on the array are closely similar (it will be appreciated that different lengths for different probes may be needed to achieve a particular T(m) where different probes have different GC contents). Although melting temperature is a primary consideration in probe design, other factors are optionally used to further adjust probe construction, such as selecting against primer self-complementarity and the like.

Arrays, e.g., microarrrays, may conveniently be stored following fabrication or purchase for use at a later time. Under suitable conditions, the subject arrays are capable of being stored for at least about 6 months and may be stored for up to one year or longer. Arrays are generally stored at temperatures between about −20° C. to room temperature, where the arrays are preferably sealed in a plastic container, e.g., bag, and shielded from light.

9.1 Hybridization of the Target Nucleic Acids to the Microarray

The next step is to contact the labeled nucleic acids with the array under conditions sufficient for binding between the probe and the target of the array. In a preferred embodiment, the probe will be contacted with the array under conditions suitable for hybridization to occur between the labeled nucleic acids and probes on the microarray, where the hybridization conditions will be selected in order to provide for the desired level of hybridization specificity.

Contact of the array and probe involves contacting the array with an aqueous medium comprising the probe. Contact may be achieved in a variety of different ways depending on specific configuration of the array. For example, where the array simply comprises the pattern of size separated targets on the surface of a “plate-like” rigid substrate, contact may be accomplished by simply placing the array in a container comprising the probe solution, such as a polyethylene bag, and the like. In other embodiments where the array is entrapped in a separation media bounded by two rigid plates, the opportunity exists to deliver the probe via electrophoretic means. Alternatively, where the array is incorporated into a biochip device having fluid entry and exit ports, the probe solution may be introduced into the chamber in which the pattern of target molecules is presented through the entry port, where fluid introduction could be performed manually or with an automated device. In multiwell embodiments, the probe solution will be introduced in the reaction chamber comprising the array, either manually, e.g., with a pipette, or with an automated fluid handling device.

Contact of the probe solution and the targets will be maintained for a suitable period of time for binding between the probe and the target to occur. Although dependent on the nature of the probe and target, contact will generally be maintained for a period of time ranging from about 10 min to 24 hrs, usually from about 30 min to 12 hrs and more usually from about 1 hr to 6 hrs.

When using commercially-available microarrays, adequate hybridization conditions are provided by the manufacturer. When using non-commercial microarrays, adequate hybridization conditions may be determined based on the following hybridization guidelines, as well as on the hybridization conditions described in the numerous published articles on the use of microarrays.

Nucleic acid hybridization and wash conditions are optimally chosen so that the probe “specifically binds” or “specifically hybridizes” to a specific array site, i.e., the probe hybridizes, duplexes or binds to a sequence array site with a complementary nucleic acid sequence but does not hybridize to a site with a non-complementary nucleic acid sequence. As used herein, one polynucleotide sequence is considered complementary to another when, if the shorter of the polynucleotides is less than or equal to 25 bases, there are no mismatches using standard base-pairing rules or, if the shorter of the polynucleotides is longer than 25 bases, there is no more than a 5% mismatch. Preferably, the polynucleotides are perfectly complementary (no mismatches). It may easily be demonstrated that specific hybridization conditions result in specific hybridization by carrying out a hybridization assay including negative controls.

Hybridization is carried out in conditions permitting essentially specific hybridization. The length of the probe and GC content will determine the T(m) of the hybrid, and thus the hybridization conditions necessary for obtaining specific hybridization of the probe to the template nucleic acid. These factors are well known to a person of skill in the art, and may also be tested in assays. An extensive guide to the hybridization of nucleic acids is found in Tijssen (1993) Laboratory Techniques in biochemistry and molecular biology-hybridization with nucleic acid probes, Elsevier, New York. Generally, stringent conditions are selected to be about 5° C. lower than the thermal melting point (T(m)) for the specific sequence at a defined ionic strength and pH. The T(m) is the temperature (under defined ionic strength and pH) at which 50% of the target sequence hybridizes to a perfectly matched probe. Highly stringent conditions are selected to be equal to the T(m) point for a particular probe. Sometimes the term “Td” is used to define the temperature at which at least half of the probe dissociates from a perfectly matched target nucleic acid. In any case, a variety of estimation techniques for estimating the T(m) or Td are available, and generally described in Tijssen, supra. Typically, G-C base pairs in a duplex are estimated to contribute about 3° C. to the T(m), while A-T base pairs are estimated to contribute about 2° C., up to a theoretical maximum of about 80-100° C. However, more sophisticated models of T(m) and Td are available and suitable in which G-C stacking interactions, solvent effects, the desired assay temperature and the like are taken into account. For example, probes may be designed to have a dissociation temperature (Td) of approximately 60° C., using the formula: Td=(((((3×#GC)+(2×#AT))×37)−562)/#bp)−5; where #GC, #AT, and #bp are the number of guanine-cytosine base pairs, the number of adenine-thymine base pairs, and the number of total base pairs, respectively, involved in the annealing of the probe to the template DNA.

The stability difference between a perfectly matched duplex and a mismatched duplex, particularly if the mismatch is only a single base, may be quite small, corresponding to a difference in T(m) between the two of as little as 0.5 degrees. See Tibanyenda, N. et al., Eur. J. Biochem. 139:19 (1984) and Ebel, S. et al., Biochem. 31:12083 (1992). More importantly, it is understood that as the length of the homology region increases, the effect of a single base mismatch on overall duplex stability decreases.

Theory and practice of nucleic acid hybridization is described, e.g., in S. Agrawal (ed.) Methods in Molecular Biology, volume 20; and Tijssen (1993) Laboratory Techniques in biochemistry and molecular biology-hybridization with nucleic acid probes, e.g., part I chapter 2 “Overview of principles of hybridization and the strategy of nucleic acid probe assays”, Elsevier, New York provide a basic guide to nucleic acid hybridization.

Certain microarrays are of “active” nature, i.e., they provide independent electronic control over all aspects of the hybridization reaction (or any other affinity reaction) occurring at each specific microlocation. These devices provide a new mechanism for affecting hybridization reactions which is called electronic stringency control (ESC). The active devices of this invention may electronically produce “different stringency conditions” at each microlocation. Thus, all hybridizations may be carried out optimally in the same bulk solution. These arrays are described, for example, in U.S. Pat. No. 6,051,380 by Sosnowski et al.

In a preferred embodiment, background signal is reduced by the use of a detergent (e.g, C-TAB) or a blocking reagent (e.g., sperm DNA, cot-1 DNA, etc.) during the hybridization to reduce non-specific binding. In a particularly preferred (embodiment, the hybridization is performed in the presence of about 0.5 mg/ml DNA (e.g., herring sperm DNA). The use of blocking agents in hybridization is well known to those of skill in the art (see, e.g., Chapter 8 in Laboratory Techniques in Biochemistry and Molecular Biology, Vol. 24: Hybridization With Nucleic Acid Probes, P. Tijssen, ed. Elsevier, N.Y., (1993)).

The method may or may not further comprise a non-bound label removal step prior to the detection step, depending on the particular label employed on the target nucleic acid. For example, in certain assay formats (e.g., “homogenous assay formats”) a detectable signal is only generated upon specific binding of target to probe. As such, in these assay formats, the hybridization pattern may be detected without a non-bound label removal step. In other embodiments, the label employed will generate a signal whether or not the target is specifically bound to its probe. In such embodiments, the non-bound labeled target is removed from the support surface. One means of removing the non-bound labeled target is to perform the well known technique of washing, where a variety of wash solutions and protocols for their use in removing non-bound label are known to those of skill in the art and may be used. Alternatively, non-bound labeled target may be removed by electrophoretic means.

Where all of the target sequences are detected using the same label, different arrays will be employed for each physiological source (where different could include using the same array at different times). The above methods may be varied to provide for multiplex analysis, by employing different and distinguishable labels for the different target populations (representing each of the different physiological sources being assayed). According to this multiplex method, the same array is used at the same time for each of the different target populations.

In another embodiment, hybridization is monitored in real time using a charge-coupled device imaging camera (Guschin et al. (1997) Anal. Biochem. 250:203). Synthesis of arrays on optical fibre bundles allows easy and sensitive reading (Healy et al. (1997) Anal. Biochem. 251:270). In another embodiment, real time hybridization detection is carried out on microarrays without washing using evanescent wave effect that excites only fluorophores that are bound to the surface (see, e.g., Stimpson et al. (1995) PNAS 92:6379).

9.2. Detection of hybridization and analysis of results

The above steps result in the production of hybridization patterns of labeled target nucleic acid on the array surface. The resultant hybridization patterns of labeled nucleic acids may be visualized or detected in a variety of ways, with the particular manner of detection being chosen based on the particular label of the target nucleic acid, where representative detection means include scintillation counting, autoradiography, fluorescence measurement, colorimetric measurement, light emission measurement, light scattering, and the like.

One method of detection includes an array scanner that is commercially available from Affymetrix, e.g., the 417™ Arrayer, the 418™ Array Scanner, or the Agilent GeneArray™ Scanner. This scanner is controlled from the system computer with a Windows^{R} interface and easy-to-use software tools. The output is a 16-bit.tif file that may be directly imported into or directly read by a variety of software applications. Preferred scanning devices are described in, e.g., U.S. Pat. Nos. 5,143,854 and 5,424,186.

When fluorescently labeled probes are used, the fluorescence emissions at each site of a transcript array may be, preferably, detected by scanning confocal laser microscopy. In one embodiment, a separate scan, using the suitable excitation line, is carried out for each of the two fluorophores used. Alternatively, a laser may be used that allows simultaneous specimen illumination at wavelengths specific to the two fluorophores and emissions from the two fluorophores may be analyzed simultaneously (see Shalon et al., 1996, A DNA microarray system for analyzing complex DNA samples using two-color fluorescent probe hybridization, Genome Research 6:639-645, which is incorporated by reference in its entirety for all purposes). In a preferred embodiment, the arrays are scanned with a laser fluorescent scanner with a computer controlled X-Y stage and a microscope objective. Sequential excitation of the two fluorophores may be achieved with a multi-line, mixed gas laser and the emitted light is split by wavelength and detected with two photomultiplier tubes. Fluorescence laser scanning devices are described in Schena et al., 1996, Genome Res. 6:639-645 and in other references cited herein. Alternatively, the fiber-optic bundle described by Ferguson et al., (1996) Nature Biotech. 14:1681-1684, may be used to monitor mRNA abundance levels.

In one embodiment in which fluorescent target nucleic acids are used, the arrays may be scanned using lasers to excite fluorescently labeled targets that have hybridized to regions of probe arrays, which may then be imaged using charged coupled devices (“CCDs”) for a wide field scanning of the array. Alternatively, another particularly useful method for gathering data from the arrays is through the use of laser confocal microscopy which combines the ease and speed of a readily automated process with high resolution detection. Particularly

Following the data gathering operation, the data will typically be reported to a data analysis operation. To facilitate the sample analysis operation, the data obtained by the reader from the device will typically be analyzed using a digital computer. Typically, the computer will be suitably programmed for receipt and storage of the data from the device, as well as for analysis and reporting of the data gathered, e.g., subtraction of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. In a preferred embodiment, a system comprises a search function that allows one to search for specific patterns, e.g., patterns relating to differential gene expression, e.g., between the expression profile of a cell of a subject having an erythropoietic disorder and the expression profile of a counterpart normal cell in a subject. A system preferably allows one to search for patterns of gene expression between more than two samples.

A desirable system for analyzing data is a general and flexible system for the visualization, manipulation, and analysis of gene expression data. Such a system preferably includes a graphical user interface for browsing and navigating through the expression data, allowing a user to selectively view and highlight the genes of interest. The system also preferably includes sort and search functions and is preferably available for general users with PC, Mac or Unix workstations. Also preferably included in the system are clustering algorithms that are qualitatively more efficient than existing ones. The accuracy of such algorithms is preferably hierarchically adjustable so that the level of detail of clustering may be systematically refined as desired.

Various algorithms are available for analyzing the gene expression profile data, e.g., the type of comparisons to perform. In certain embodiments, it is desirable to group genes that are co-regulated. This allows the comparison of large numbers of profiles. A preferred embodiment for identifying such groups of genes involves clustering algorithms (for reviews of clustering algorithms, see, e.g., Fukunaga, 1990, Statistical Pattern Recognition, 2nd Ed., Academic Press, San Diego; Everitt, 1974, Cluster Analysis, London: Heinemann Educ. Books; Hartigan, 1975, Clustering Algorithms, New York: Wiley; Sneath and Sokal, 1973, Numerical Taxonomy, Freeman; Anderberg, 1973, Cluster Analysis for Applications, Academic Press: New York).

Clustering analysis is useful in helping to reduce complex patterns of thousands of time curves into a smaller set of representative clusters. Some systems allow the clustering and viewing of genes based on sequences. Other systems allow clustering based on other characteristics of the genes, e.g., their level of expression (see, e.g., U.S. Pat. No. 6,203,987). Other systems permit clustering of time curves (see, e.g. U.S. Pat. No. 6,263,287). Cluster analysis may be performed using the hclust routine (see, e.g., “hclust” routine from the software package S-Plus, MathSoft, Inc., Cambridge, Mass.).

In some specific embodiments, genes are grouped according to the degree of co-variation of their transcription, presumably co-regulation, as described, for example, in U.S. Pat. No. 6,203,987. Groups of genes that have co-varying transcripts are termed “genesets.” Cluster analysis or other statistical classification methods may be used to analyze the co-variation of transcription of genes in response to a variety of perturbations, e.g. caused by a disease or a drug. In one specific embodiment, clustering algorithms are applied to expression profiles to construct a “similarity tree” or “clustering tree” which relates genes by the amount of co-regulation exhibited. Genesets are defined on the branches of a clustering tree by cutting across the clustering tree at different levels in the branching hierarchy.

In some embodiments, a gene expression profile is converted to a projected gene expression profile. The projected gene expression profile is a collection of geneset expression values. The conversion is achieved, in some embodiments, by averaging the level of expression of the genes within each geneset. In some other embodiments, other linear projection processes may be used. The projection operation expresses the profile on a smaller and biologically more meaningful set of coordinates, reducing the effects of measurement errors by averaging them over each cellular constituent sets and aiding biological interpretation of the profile.

10. Diagnostics and Prognostics for Lung Cancer

The present invention provides methods of diagnosing lung cancer. The present invention also provides prognostic methods for evaluating the progression of lung cancer or the outcome of therapy directed toward lung cancer. The invention provides panels of genes identified via gene expression profiling as being involved in the neoplasia of lung cells. The genes, which are up- or downregulated in lung cell neoplasia, are referred to herein as “genes involved in lung cell neoplasia”. Accordingly, the expression profiles of the genes in the panel may be used diagnostically and prognostically for lung cancer. Exemplary diagnostic tools and assays are set forth below, under (i) to (vi), followed by exemplary methods for conducting these assays. The assays may optionally utilize the microarrays of the invention.

(i) In one embodiment, the invention provides methods for determining whether a subject has or is likely to develop lung cancer, comprising determining the level of expression of one or more genes which are up- or downregulated during lung cell neoplasia in a cell of the subject and comparing these levels of expression with the levels of expression of the genes in a diseased cell of a subject known to have lung cancer, such that a similar level of expression of the genes is indicative that the subject has or is likely to develop lung cancer or at least a symptom thereof. In a preferred embodiment, the cell is essentially of the same type as that which is diseased in the subject.

(ii) In another embodiment the expression profiles of genes in the panels of the invention may be used to confirm that a subject has a specific type of lung cancer, and in particular, that the subject does not have a related disease or disease with similar symptoms. This may be important, in particular, in designing an optimal therapeutic regimen for the subject. It has been described in the art that expression profiles may be used to distinguish one type of disease from a similar disease. For example, two subtypes of non-Hodgkin's lymphomas, one of which responds to current therapeutic methods and the other one which does not, could be differentiated by investigating 17,856 genes in specimens of patients suffering from diffuse large B-cell lymphoma (Alizadeh et al. Nature (2000) 405:503). Similarly, subtypes of cutaneous melanoma were predicted based on profiling 8150 genes (Bittner et al. Nature (2000) 406:536). In this case, features of the highly aggressive metastatic melanomas could be recognized. Numerous other studies comparing expression profiles of cancer cells and normal cells have been described, including studies describing expression profiles distinguishing between highly and less metastatic cancers and studies describing new subtypes of diseases, e.g., new tumor types (see, e.g., Perou et al. (1999) PNAS 96: 9212; Perou et al. (2000) Nature 606:747; Clark et al. (2000) Nature 406:532; Alon et al. (1999) PNAS 96:6745; Golub et al. (1999) Science 286:531).

Accordingly, the expression profiles of the invention allow the distinction of lung cancer from related diseases. Such distinction is known in the art as “differential diagnosis”. In a preferred embodiment, the level of expression of one or more genes whose expression is characteristic of lung cancer is determined in a cell of the subject. In an even more preferred embodiment, the level of expression of essentially all of the genes involved in neoplasia of lung cells is determined in a cell of the subject, such as by using a microarray comprising probes corresponding to all of or essentially all of the genes identified in FIG. 2. A level of expression of one or more genes involved in lung cancer in the a cell of a first subject that is similar to the level of expression of the same genes in a cell of a reference subject known to have lung cancer indicates that the first subject has lung cancer, rather than a disease related to or similar to lung cancer.

Prior to using this method for determining whether the subject has lung cancer or a related disease, it may be necessary to first determine the expression profile of cells of diseases that are similar to lung cancer and cells from numerous subjects having lung cancer as diagnosed by traditional (i.e., non microarray based) methods. This may be undertaken using a microarray containing the panel of genes involved in lung cell neoplasia according to methods further described herein.

(iii) In yet another embodiment, the invention provides methods for determining the stage of a lung cancer in the subject. It is thought that the level of expression of the genes that are characteristic of lung cancer changes with the stage of the disease. This could be confirmed, e.g., by analyzing the level of expression of these genes in subjects having lung cancer at different stages, as determined by traditional methods. For example, the expression profile of a diseased cell in subjects at different stages of the disease may be determined as described herein. Then, to determine the stage of lung cancer in a subject, the level of expression of one or more genes that are characteristic of the disorder and whose level of expression varies with the stage of the disease is determined. A similar level of expression of one or more genes whose expression is characteristic of a lung cancer between that in a subject and that in a reference profile of a particular stage of the disease, indicates that the lung cancer of the subject is at the particular stage.

(iv) Similarly, the methods may used to determine the stage of the disease in a subject undergoing therapy, and thereby determine whether the therapy is effective. Accordingly, in one embodiment, the level of expression of one or more genes involved in lung cell neoplasia is determined in a subject before the treatment and several times during the treatment. For example, a sample of RNA may be obtained from the subject before the beginning of the therapy and every 12, 24 or 72 hours during the therapy. Samples may also be analyzed one a week or once a month. Changes in expression levels of genes whose expression is characteristic of lung cell pathogenesis over time and relative to diseased cells and normal cells will indicate whether the therapy is effective.

(v) In yet another embodiment, the invention provides methods for determining the likelihood of success of a particular therapy in a subject having lung cancer. In one embodiment, a subject is started on a particular therapy, and the effectiveness of the therapy is determined, e.g., by determining the level of expression of one or more genes whose expression is characteristic of lung cancer in a cell of the subject. A normalization of the level of expression of these genes, i.e., a change in the expression level of the genes such that their level of expression resembles more that of a non diseased cell, indicates that the treatment should be effective in the subject.

Prediction of the outcome of a treatment of lung cancer in a subject may also be undertaken in vitro. In one embodiment, cells are obtained from a subject to be evaluated for responsiveness to the treatment, and incubated in vitro with the therapeutic drug. The level of expression of one or more genes involved in neoplasia of lung cells is then measured in the cells and these values are compared to the level of expression of these one or more genes in a cell which is the normal counterpart cell of a diseased cell. The level of expression may also be compared to that in a normal cell. In a preferred embodiment, the level of expression of essentially all the genes whose expression is characteristic of lung cancer, i.e., the genes shown in FIGS. 2, 3, and 4, or TrkB or Aur2 is determined. The comparative analysis is preferably conducted using a computer comprising a database comprising the level of expression of at least one gene characteristic of lung cancer in a diseased and/or normal cell. A level of expression of one or more genes whose expression is characteristic of lung cancer in the cells of the subject after incubation with the drug that is similar to their level of expression in a normal cell and different from that in a diseased cell is indicative that it is likely that the subject will respond positively to a treatment with the drug. On the contrary, a level of expression of one or more genes whose expression is characteristic of lung cancer in the cells of the subject after incubation with the drug that is similar to their level of expression in a diseased cell and different from that in a normal cell is indicative that it is likely that the subject will not respond positively to a treatment with the drug.

Since it is possible that a drug for treating lung cancer does not act directly on the diseased cells, but is, e.g., metabolized, or acts on another cell which then secretes a factor that will effect the diseased cells, the above assay may also be conducted in a tissue sample of a subject, which contains cells other than the diseased cells. For example, a tissue sample comprising diseased cells is obtained from a subject; the tissue sample is incubated with the potential drug; optionally one or more diseased cells are isolated from the tissue sample, e.g., by microdissection or Laser Capture Microdissection (LCM, see infra); and the expression level of one or more genes whose expression is characteristic of lung cancer is examined.

(vi) The invention may also provide methods for selecting a therapy for lung cancer for a patient from a selection of several different treatments. Certain subjects having lung cancer may respond better to one type of therapy than another type of therapy. In a preferred embodiment, the method comprises comparing the expression level of at least one gene characteristic of lung cancer in the patient with that in cells of subjects treated in vitro or in vivo with one of several therapeutic drugs, which subjects are responders or non responders to one of the therapeutic drugs, and identifying the cell which has the most similar level of expression of the one or more genes to that of the patient, to thereby identify a therapy for the patient. The method may further comprise administering the therapy identified to the subject.

A person of skill in the art will appreciate that in some embodiments of diagnostic and prognostic assays, it will be desirable to assess the level of expression of a single gene characteristic of lung cancer and that in others, the expression of two or more is preferred, whereas still in others, the expression of essentially all the genes involved in lung cell neoplasia is preferably assessed.

Set forth below are exemplary methods which may be used to determine the level of expression of one or more genes involved in lung cell neoplasia, e.g., for use in the above-described methods. For example, the level of expression of a gene may be determined by reverse transcription-polymerase chain reaction (RT-PCR); dot blot analysis; Northern blot analysis and in situ hybridization. In a preferred embodiment, the level of expression is determined by using a microarray which contains probes of the genes that are up- or down-regulated during lung cell neoplasia. In another embodiment, the level of protein encoded by one or more of the genes that are up- or down-regulated during lung cell neoplasia is determined in a cell of the type that is diseased in. This may be done by a variety of methods, e.g., immunohistochemistry.

10.1. Use of Microarrays for Determining the Level of Expression of Genes Whose Expression is Characteristic of a Lung Cancer

Generally, determining expression profiles with microarrays involves the following steps: (a) obtaining a mRNA sample from a subject and preparing labeled nucleic acids therefrom (the “target nucleic acids” or “targets”); (b) contact of the target nucleic acids with the array under conditions sufficient for target nucleic acids to bind with corresponding probe on the array, e.g., by hybridization or specific binding; (c) optional removal of unbound targets from the array; and (d) detection of bound targets, and analysis of the results, e.g., using computer based analysis methods. As used herein, “nucleic acid probes” or “probes” are nucleic acids attached to the array, whereas “target nucleic acids” are nucleic acids that are hybridized to the array. Each of these steps is described in more detail below.

(i) Obtaining a mRNA Sample of a Subject

Nucleic acid specimens may be obtained from an individual to be tested using either “invasive” or “non-invasive” sampling means. A sampling means is said to be “invasive” if it involves the collection of nucleic acids from within the skin or organs of an animal (including, especially, a murine, a human, an ovine, an equine, a bovine, a porcine, a canine, or a feline animal). Examples of invasive methods include blood collection, semen collection, needle biopsy, pleural aspiration, umbilical cord biopsy, etc. Examples of such methods are discussed by Kim, C. H. et al. (1992) J. Virol. 66:3879-3882; Biswas, B. et al. (1990) Annals NY Acad. Sci. 590:582-583; Biswas, B. et al. (1991) J. Clin. Microbiol. 29:2228-2233.

In one embodiment, one or more cells from the subject to be tested are obtained and RNA is isolated from the cells. In a preferred embodiment, a sample of lung cell s is obtained from the subject. When obtaining the cells, it is preferable to obtain a sample containing predominantly cells of the desired type, e.g., a sample of cells in which at least about 50%, preferably at least about 60%, even more preferably at least about 70%, 80% and even more preferably, at least about 90% of the cells are of the desired type. A higher percentage of cells of the desired type is preferable, since such a sample is more likely to provide clear gene expression data. Blood samples may be obtained according to methods known in the art.

It is also possible to obtain a cell sample from a subject, and then to enrich it in the desired cell type. For example, cells may be isolated from other cells using a variety of techniques, such as isolation with an antibody binding to an epitope on the cell surface of the desired cell type.

In one embodiment, RNA is obtained from a single cell. It is also possible to obtain cells from a subject and culture the cells in vitro, such as to obtain a larger population of cells from which RNA may be extracted. Methods for establishing cultures of non-transformed cells, i.e., primary cell cultures, are known in the art.

When isolating RNA from tissue samples or cells from individuals, it may be important to prevent any further changes in gene expression after the tissue or cells has been removed from the subject. Changes in expression levels are known to change rapidly following perturbations, e.g., heat shock or activation with lipopolysaccharide (LPS) or other reagents. In addition, the RNA in the tissue and cells may quickly become degraded. Accordingly, in a preferred embodiment, the cells obtained from a subject are snap frozen as soon as possible.

RNA may be extracted from the tissue sample by a variety of methods, e.g., the guanidium thiocyanate lysis followed by CsCl centrifugation (Chirgwin et al., (1979), Biochemistry 18:5294-5299). RNA from single cells may be obtained as described in methods for preparing cDNA libraries from single cells, such as those described in Dulac, C. (1998) Curr. Top. Dev. Biol. 36, 245 and Jena et al. (1996) J. Immunol. Methods 190:199. Care to avoid RNA degradation must be taken, e.g., by inclusion of RNAsin.

The RNA sample may then be enriched in particular species. In one embodiment, poly(A)+ RNA is isolated from the RNA sample. In general, such purification takes advantage of the poly-A tails on mRNA. In particular and as noted above, poly-T oligonucleotides may be immobilized within on a solid support to serve as affinity ligands for mRNA. Kits for this purpose are commercially available, e.g., the MessageMaker kit (Life Technologies, Grand Island, N.Y.).

In a preferred embodiment, the RNA population is enriched in sequences of interest, such as those of the genes involved in lung cell neoplasia. Enrichment may be undertaken, e.g., by primer-specific cDNA synthesis, or multiple rounds of linear amplification based on cDNA synthesis and template-directed in vitro transcription (see, e.g., Wang et al. (1989) PNAS 86, 9717; Dulac et al., supra, and Jena et al., supra).

The population of RNA, enriched or not in particular species or sequences, may further be amplified. Such amplification is particularly important when using RNA from a single or a few cells. A variety of amplification methods are suitable for use in the methods of the invention, including, e.g., PCR; ligase chain reaction (LCR) (see, e.g., Wu and Wallace, (1989) Genomics 4, 560, Landegren et al. (1988) Science 241, 1077); self-sustained sequence replication (SSR) (see, e.g., Guatelli et al., (1990) PNAS, 87, 1874); nucleic acid based sequence amplification (NASBA) and transcription amplification (see, e.g., Kwoh et al.,(1989) PNAS 86, 1173). For PCR technology, see, e.g., PCR Technology: Principles and Applications for DNA Amplification (ed. H. A. Erlich, Freeman Press, N.Y., N.Y., 1992); PCR Protocols: A Guide to Methods and applications (eds. Innis, et al., Academic Press, San Diego, Calif., 1990); Mattila et al., (1991) Nucleic Acids Res. 19, 4967; Eckert et al., PCR Methods and Applications 1, 17 (1991); PCR (eds. McPherson et al., IRL Press, Oxford); and U.S. Pat. No. 4,683,202. Methods of amplification are described, e.g., in Ohyama et al. (2000) BioTechniques 29:530; Luo et al. (1999) Nat. Med. 5, 117; Hegde et al. (2000) BioTechniques 29:548; Kacharmina et al. (1999) Meth. Enzymol. 303:3; Livesey et al. (2000) Curr. Biol. 10:301; Spirin et al. (1999) Invest. Ophtalmol. Vis. Sci. 40:3108; and Sakai et al. (2000) Anal. Biochem. 287:32. RNA amplification and cDNA synthesis may also be conducted in cells in situ (see, e.g., Eberwine et al. (1992) PNAS 89:3010).

One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. A high density array may then include probes specific to the internal standard for quantification of the amplified nucleic acid.

One preferred internal standard is a synthetic AW106 RNA. The AW106 RNA is combined with RNA isolated from the sample according to standard techniques known to those of skilled in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of radioactivity (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al., Academic Press, Inc. N.Y., (1990).

In a preferred embodiment, a sample mRNA is reverse transcribed with a reverse transcriptase and a primer consisting of oligo(dT) and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template results in amplified RNA. Methods of in vitro polymerization are well known to those of skill in the art (see, e.g., Sambrook, (supra) and this particular method is described in detail by Van Gelder, et al., (1990) PNAS, 87: 1663-1667 who demonstrate that in vitro amplification according to this method preserves the relative frequencies of the various RNA transcripts. Moreover, Eberwine et al. PNAS, 89: 3010-3014 provide a protocol that uses two rounds of amplification via in vitro transcription to achieve greater than 10⁶ fold amplification of the original starting material, thereby permitting expression monitoring even where biological samples are limited.

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

(ii) Labeling of the Nucleic Acids to be Analyzed

Generally, the target molecules will be labeled to permit detection of hybridization of target molecules to a microarray. By labeled is meant that the probe comprises a member of a signal producing system and is thus detectable, either directly or through combined action with one or more additional members of a signal producing system. Examples of directly detectable labels include isotopic and fluorescent moieties incorporated into, usually covalently bonded to, a moiety of the probe, such as a nucleotide monomeric unit, e.g., dNMP of the primer, or a photoactive or chemically active derivative of a detectable label which may be bound to a functional moiety of the probe molecule.

Nucleic acids may be labeled after or during enrichment and/or amplification of RNAs. For example, labeled cDNA is prepared from mRNA by oligo dT-primed or random-primed reverse transcription, both of which are well known in the art (see, e.g., Klug and Berger, (1987) Methods Enzymol. 152:316-325). Reverse transcription may be carried out in the presence of a dNTP conjugated to a detectable label, most preferably a fluorescently labeled dNTP. Alternatively, isolated mRNA may be converted to labeled antisense RNA synthesized by in vitro transcription of double-stranded cDNA in the presence of labeled dNTPs (Lockhart et al. (1996) Nature Biotech. 14:1675, which is incorporated by reference in its entirety for all purposes). In alternative embodiments, the cDNA or RNA probe may be synthesized in the absence of detectable label and may be labeled subsequently, e.g., by incorporating biotinylated dNTPs or rNTP, or some similar means (e.g., photo-cross-linking a psoralen derivative of biotin to RNAs), followed by addition of labeled streptavidin (e.g., phycoerythrin-conjugated streptavidin) or the equivalent.

In one embodiment, labeled cDNA is synthesized by incubating a mixture containing 0.5 mM dGTP, dATP and dCTP plus 0.1 mM dTTP plus fluorescent deoxyribonucleotides (e.g., 0.1 mM rhodamine 110 UTP (Perkin Elmer Cetus, Mass.) or 0.1 mM Cy3 dUTP (Amersham, N.J.) with reverse transcriptase (e.g., SuperScript.™.II, LTI Inc., CA) at 42° C. for 60 min.

Fluorescent moieties or labels of interest include coumarin and its derivatives, e.g., 7-amino-4-methylcoumarin, aminocoumarin, bodipy dyes, such as BODIPY® FL, cascade blue, fluorescein and its derivatives, e.g., fluorescein isothiocyanate, Oregon green, rhodamine dyes, e.g., Texas red, tetramethylrhodamine, eosins and erythrosins, cyanine dyes, e.g., Cy2, Cy3, Cy3.5, Cy5, Cy5.5, Cy7, FluorX, macrocyclic chelates of lanthanide ions, e.g., quantum dye™, fluorescent energy transfer dyes, such as thiazole orange-ethidium heterodimer, TOTAB, dansyl, etc. Individual fluorescent compounds which have functionalities for linking to an element desirably detected in an apparatus or assay of the invention, or which may be modified to incorporate such functionalities include, e.g., dansyl chloride; fluoresceins such as 3,6-dihydroxy-9-phenylxanthydrol; rhodamineisothiocyanate; N-phenyl 1-amino-8-sulfonatonaphthalene; N-phenyl 2-amino-6-sulfonatonaphthalene; 4-acetamido-4-isothiocyanatostilbene-2,2′-disulfonic acid; pyrene-3-sulfonic acid; 2-toluidinonaphthalene-6-sulfonate; N-phenyl-N-methyl-2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4-(3′-pyrenyl)stearate; d-3-aminodesoxy-equilenin; 12-(9′-anthroyl)stearate; 2-methylanthracene; 9-vinylanthracene; 2,2′(vinylene-p-phenylene)bisbenzoxazole; p-bis(2-methyl-5-phenyl-oxazolyl))benzene; 6-dimethylamino-1,2-benzophenazin; retinol; bis(3′-aminopyridinium) 1,10-decandiyl diiodide; sulfonaphthylhydrazone of hellibrienin; chlorotetracycline; N-(7-dimethylamino-4-methyl-2-oxo-3-chromenyl)maleimide; N-(p-(2benzimidazolyl)-phenyl)maleimide; N-(4-fluoranthyl)maleimide; bis(homovanillic acid); resazarin; 4-chloro-7-nitro-2,1,3-benzooxadiazole; merocyanine 540; resorufin; rose bengal; and 2,4-diphenyl-3(2H)-forenoon. (see, e.g., Kricka, (1992) Nonisotopic DNA Probe Techniques, Academic Press San Diego, Calif.). Many fluorescent tags are commercially available from SIGMA chemical company (Saint Louis, Mo.), Amersham, Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Aldrich Chemical Company (Milwaukee, Wis.), GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.) as well as other commercial sources known to one of skill in the art.

Chemiluminescent labels include luciferin and 2,3-dihydrophthalazinediones, e.g., luminol.

Isotopic moieties or labels of interest include ³²P, ³³P, ³⁵S, ¹²⁵I, ²H, ¹⁴C, and the like (see Zhao et al., 1995, High density cDNA filter analysis: a novel approach for large-scale, quantitative analysis of gene expression (Pietu et al. (1996) Gene 156:20 and Genome Res. 6:492). However, because of scattering of radioactive particles, and the consequent requirement for widely spaced binding sites, use of radioisotopes is a less-preferred embodiment.

Labels may also be members of a signal producing system that act in concert with one or more additional members of the same system to provide a detectable signal. Illustrative of such labels are members of a specific binding pair, such as ligands, e.g., biotin, fluorescein, digoxigenin, antigen, polyvalent cations, chelator groups and the like, where the members specifically bind to additional members of the signal producing system, where the additional members provide a detectable signal either directly or indirectly, e.g., antibody conjugated to a fluorescent moiety or an enzymatic moiety capable of converting a substrate to a chromogenic product, e.g., alkaline phosphatase conjugate antibody and the like.

Additional labels of interest include those that provide for signal only when the probe with which they are associated is specifically bound to a target molecule, where such labels include: “molecular beacons” as described in Tyagi & Kramer, (1996) Nature Biotechnol. 14:303 and EP 0 070 685 B1. Other labels of interest include those described in U.S. Pat. No. 5,563,037; WO 97/17471 and WO 97/17076.

In some cases, hybridized target nucleic acids may be labeled following hybridization. For example, where biotin labeled dNTPs are used in, e.g., amplification or transcription, streptavidin linked reporter groups may be used to label hybridized complexes.

In other embodiments, the target nucleic acid is not labeled. In this case, hybridization may be determined, e.g., by plasmon resonance, as described, e.g., in Thiel et al. (1997) Anal. Chem. 69:4948.

In one embodiment, a plurality (e.g., 2, 3, 4, 5 or more) of sets of target nucleic acids are labeled and used in one hybridization reaction (“multiplex” analysis). For example, one set of nucleic acids may correspond to RNA from one cell and another set of nucleic acids may correspond to RNA from another cell. The plurality of sets of nucleic acids may be labeled with different labels, e.g., different fluorescent labels which have distinct emission spectra so that they may be distinguished. The sets may then be mixed and hybridized simultaneously to one microarray.

For example, the two different cells may be a diseased lung cell and a counterpart normal cell. Alternatively, the two different cells may be a diseased lung cell of a patient having lung cancer and a diseased lung cell of a patient suspected of having lung cancer. In another embodiment, one biological sample is exposed to a drug and another biological sample of the same type is not exposed to the drug. The cDNA derived from each of the two cell types are differently labeled so that they may be distinguished. In one embodiment, for example, cDNA from a diseased cell is synthesized using a fluorescein-labeled dNTP, and cDNA from a second cell, i.e., the normal cell, is synthesized using a rhodamine-labeled dNTP. When the two cDNAs are mixed and hybridized to the microarray, the relative intensity of signal from each cDNA set is determined for each site on the array, and any relative difference in abundance of a particular mRNA detected.

In the example described above, the cDNA from the diseased lug cell will fluoresce green when the fluorophore is stimulated and the cDNA from the cell of a subject suspected of having lung cancer will fluoresce red. As a result, if the two cells are essentially the same, the particular mRNA will be equally prevalent in both cells and, upon reverse transcription, red-labeled and green-labeled cDNA will be equally prevalent. When hybridized to the microarray, the binding site(s) for that species of RNA will emit wavelengths characteristic of both fluorophores (and appear brown in combination). In contrast, if the two cells are different, the ratio of green to red fluorescence will be different.

The use of a two-color fluorescence labeling and detection scheme to define alterations in gene expression has been described, e.g., in Shena et al., (1995) Science 270:467-470. An advantage of using cDNA labeled with two different fluorophores is that a direct and internally controlled comparison of the mRNA levels corresponding to each arrayed gene in two cell states may be made, and variations due to minor differences in experimental conditions (e.g, hybridization conditions) will not affect subsequent analyses.

Examples of distinguishable labels for use when hybridizing a plurality of target nucleic acids to one array are well known in the art and include: two or more different emission wavelength fluorescent dyes, like Cy3 and Cy5, combination of fluorescent proteins and dyes, like phicoerythrin and Cy5, two or more isotopes with different energy of emission, like ³²P and ³³P, gold or silver particles with different scattering spectra, labels which generate signals under different treatment conditions, like temperature, pH, treatment by additional chemical agents, etc., or generate signals at different time points after treatment. Using one or more enzymes for signal generation allows for the use of an even greater variety of distinguishable labels, based on different substrate specificity of enzymes (alkaline phosphatase/peroxidase).

Further, it is preferable in order to reduce experimental error to reverse the fluorescent labels in two-color differential hybridization experiments to reduce biases peculiar to individual genes or array spot locations. In other words, it is preferable to first measure gene expression with one labeling (e.g., labeling nucleic acid from a first cell with a first fluorochrome and nucleic acid from a second cell with a second fluorochrome) of the mRNA from the two cells being measured, and then to measure gene expression from the two cells with reversed labeling (e.g., labeling nucleic acid from the first cell with the second fluorochrome and nucleic acid from the second cell with the first fluorochrome). Multiple measurements over exposure levels and perturbation control parameter levels provide additional experimental error control.

The quality of labeled nucleic acids may be evaluated prior to hybridization to an array. For example, a sample of the labeled nucleic acids may be hybridized to probes derived from the 5′, middle and 3′ portions of genes known to be or suspected to be present in the nucleic acid sample. This will be indicative as to whether the labeled nucleic acids are full length nucleic acids or whether they are degraded. In one embodiment, the GeneChip® Test3 Array from Affymetrix may be used for that purpose. This array contains probes representing a subset of characterized genes from several organisms including mammals. Thus, the quality of a labeled nucleic acid sample may be determined by hybridization of a fraction of the sample to an array, such as the GeneChip® Test3 Array from Affymetrix.

10.2. Other Methods for Determining Gene Expression Levels

In certain embodiments, it is sufficient to determine the expression of one or only a few genes, as opposed to hundreds or thousands of genes. Although microarrays may be used in these embodiments, various other methods of detection of gene expression are available. This section describes a few exemplary methods for detecting and quantifying mRNA or polypeptide encoded thereby. Where the first step of the methods includes isolation of mRNA from cells, this step may be conducted as described above. Labeling of one or more nucleic acids may be performed as described above.

In one embodiment, mRNA obtained form a sample is reverse transcribed into a first cDNA strand and subjected to PCR, e.g., RT-PCR. House keeping genes, or other genes whose expression does not vary may be used as internal controls and controls across experiments. Following the PCR reaction, the amplified products may be separated by electrophoresis and detected. By using quantitative PCR, the level of amplified product will correlate with the level of RNA that was present in the sample. The amplified samples may also be separated on a agarose or polyacrylamide gel, transferred onto a filter, and the filter hybridized with a probe specific for the gene of interest. Numerous samples may be analyzed simultaneously by conducting parallel PCR amplification, e.g., by multiplex PCR.

“Dot blot” hybridization has gained wide-spread use, and many versions were developed (see, e.g., M. L. M. Anderson and B. D. Young, in Nucleic Acid Hybridization-A Practical Approach, B. D. Hames and S. J. Higgins, Eds., IRL Press, Washington D.C., Chapter 4, pp. 73-111, 1985).

In another embodiment, mRNA levels is determined by dot blot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985). In one embodiment, a specified amount of RNA extracted from cells is blotted (i.e., non-covalently bound) onto a filter, and the filter is hybridized with a probe of the gene of interest. Numerous RNA samples may be analyzed simultaneously, since a blot may comprise multiple spots of RNA. Hybridization is detected using a method that depends on the type of label of the probe. In another dot blot method, one or more probes of one or more genes whose expression is characteristic of lung cancer are attached to a membrane, and the membrane is incubated with labeled nucleic acids obtained from and optionally derived from RNA of a cell or tissue of a subject. Such a dot blot is essentially an array comprising fewer probes than a microarray.

Another format, the so-called “sandwich” hybridization, involves covalently attaching oligonucleotide probes to a solid support and using them to capture and detect multiple nucleic acid targets (see, e.g., M. Ranki et al. (1983) Gene, 21:77-85; A. M. Palva, et al, in UK Patent Application GB 2156074A, Oct. 2, 1985; T. M. Ranki and H. E. Soderlund in U.S. Pat. No. 4,563,419, Jan. 7, 1986; A. D. B. Malcolm and J. A. Langdale, in PCT WO 86/03782, Jul. 3, 1986; Y. Stabinsky, in U.S. Pat. No. 4,751,177, Jan. 14, 1988; T. H. Adams et al., in PCT WO 90/01564, Feb. 22, 1990; R. B. Wallace et al. (1979) Nucleic Acid Res. 6,11:3543; and B. J. Connor et al. (1983) PNAS 80:278-282). Multiplex versions of these formats are called “reverse dot blots.”

mRNA levels may also be determined by Northern blots. Specific amounts of RNA are separated by gel electrophoresis and transferred onto a filter which is then hybridized with a probe corresponding to the gene of interest. This method, although more burdensome when numerous samples and genes are to be analyzed provides the advantage of being very accurate.

A preferred method for high throughput analysis of gene expression is the serial analysis of gene expression (SAGE) technique, first described in Velculescu et al. (1995) Science 270, 484-487. Among the advantages of SAGE is that it has the potential to provide detection of all genes expressed in a given cell type, provides quantitative information about the relative expression of such genes, permits ready comparison of gene expression of genes in two cells, and yields sequence information that may be used to identify the detected genes. Thus far, SAGE methodology has proved itself to reliably detect expression of regulated and nonregulated genes in a variety of cell types (Velculescu et al. (1997) Cell 88, 243-251; Zhang et al. (1997) Science 276, 1268-1272 and Velculescu et al. (1999) Nat. Genet. 23, 387-388.

Techniques for producing and probing nucleic acids are further described, for example, in Sambrook et al., Molecular Cloning: A Laboratory Manual (New York, Cold Spring Harbor Laboratory, 1989).

Alternatively, the level of expression of one or more genes involved in pathogenesis of lung cells is determined by in situ hybridization. In one embodiment, a tissue sample is obtained from a subject, the tissue sample is sliced, and in situ hybridization is performed according to methods known in the art, to determine the level of expression of the genes of interest.

In other methods, the level of expression of a gene is detected by measuring the level of protein encoded by the gene. This may be done, e.g., by immunoprecipitation, ELISA, or immunohistochemistry using an agent, e.g., an antibody, that specifically detects the protein encoded by the gene. Other techniques include Western blot analysis. Immunoassays are commonly used to quantitate the levels of proteins in cell samples, and many other immunoassay techniques are known in the art. The invention is not limited to a particular assay procedure, and therefore is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In the case of polypeptides which are secreted from cells, the level of expression of these polypeptides may be measured in biological fluids.

10.3. Data Analysis Methods

Comparison of the expression levels of one or more genes involved in lung cell neoplasia with reference expression levels, e.g., expression levels in diseased lung cells of a subject having lung cancer or in normal counterpart cells, is preferably conducted using computer systems. In one embodiment, expression levels are obtained in two cells and these two sets of expression levels are introduced into a computer system for comparison. In a preferred embodiment, one set of expression levels is entered into a computer system for comparison with values that are already present in the computer system, or in computer-readable form that is then entered into the computer system.

In one embodiment, the invention provides computer readable forms of the gene expression profile data of the invention, or of values corresponding to the level of expression of at least one gene involved in lung cell neoplasia in a diseased cell. The values may be mRNA expression levels obtained from experiments, e.g., microarray analysis. The values may also be mRNA levels normalized relative to a reference gene whose expression is constant in numerous cells under numerous conditions. In other embodiments, the values in the computer are ratios of, or differences between, normalized or non-normalized mRNA levels in different samples.

The gene expression profile data may be in the form of a table, such as an Excel table. The data may be alone, or it may be part of a larger database, e.g., comprising other expression profiles. For example, the expression profile data of the invention may be part of a public database. The computer readable form may be in a computer. In another embodiment, the invention provides a computer displaying the gene expression profile data.

In one embodiment, the invention provides methods for determining the similarity between the level of expression of one or more genes involved in lung cell neoplasia in a first cell, e.g., a cell of a subject, and that in a second cell, comprising obtaining the level of expression of one or more genes involved in lung cell neoplasia in a first cell and entering these values into a computer comprising a database including records comprising values corresponding to levels of expression of one or more genes whose expression is characteristic of lung cancer in a second cell, and processor instructions, e.g., a user interface, capable of receiving a selection of one or more values for comparison purposes with data that is stored in the computer. The computer may further comprise a means for converting the comparison data into a diagram or chart or other type of output.

In another embodiment, values representing expression levels of genes involved in lung cell neoplasia are entered into a computer system, comprising one or more databases with reference expression levels obtained from more than one cell. For example, a computer may comprise expression data of diseased and normal cells. Instructions are provided to the computer, and the computer is capable of comparing the data entered with the data in the computer to determine whether the data entered is more similar to that of a normal cell or of a diseased cell.

In another embodiment, the computer comprises values of expression levels in cells of subjects at different stages of cancer and the computer is capable of comparing expression data entered into the computer with the data stored, and produce results indicating to which of the expression profiles in the computer, the one entered is most similar, such as to determine the stage of lung cancer in the subject.

In yet another embodiment, the reference expression profiles in the computer are expression profiles from cells of one or more subjects having lung cancer, which cells are treated in vivo or in vitro with a drug used for therapy of lung cancer. Upon entering of expression data of a cell of a subject treated in vitro or in vivo with the drug, the computer is instructed to compare the data entered to the data in the computer, and to provide results indicating whether the expression data input into the computer are more similar to those of a cell of a subject that is responsive to the drug or more similar to those of a cell of a subject that is not responsive to the drug. Thus, the results indicate whether the subject is likely to respond to the treatment with the drug or unlikely to respond to it.

In one embodiment, the invention provides systems comprising a means for receiving gene expression data for one or a plurality of genes; a means for comparing the gene expression data from each of said one or plurality of genes to a common reference frame; and a means for presenting the results of the comparison. A system may further comprise a means for clustering the data.

In another embodiment, the invention provides computer programs for analyzing gene expression data comprising (a) a computer code that receives as input gene expression data for a plurality of genes and (b) a computer code that compares said gene expression data from each of said plurality of genes to a common reference frame.

The invention also provides machine-readable or computer-readable media including program instructions for performing the following steps: (a) comparing a plurality of values corresponding to expression levels of one or more genes involved in the neoplasia of lung cells in a query cell with a database including records comprising reference expression or expression profile data of one or more reference cells and an annotation of the type of cell; and (b) indicating to which cell the query cell is most similar based on similarities of expression profiles. The reference cells may be cells from subjects at different stages of lung cancer. The reference cells may also be cells from subjects responding or not responding to a particular drug treatment and optionally incubated in vitro or in vivo with the drug.

The reference cells may also be cells from subjects responding or not responding to several different treatments, and the computer system indicates a preferred treatment for the subject. Accordingly, the invention provides methods for selecting a therapy for a patient having lung cancer; the methods comprising: (a) providing the level of expression of one or more genes involved in neoplasia in a diseased cell of the patient; (b) providing a plurality of reference profiles, each associated with a therapy, wherein the subject expression profile and each reference profile has a plurality of values, each value representing the level of expression of a gene involved in the neoplasia of lung cells; and (c) selecting the reference profile most similar to the subject expression profile, to thereby select a therapy for said patient. In a preferred embodiment step (c) is performed by a computer. The most similar reference profile may be selected by weighing a comparison value of the plurality using a weight value associated with the corresponding expression data.

The relative abundance of a mRNA in two biological samples may be scored as a perturbation and its magnitude determined (i.e., the abundance is different in the two sources of mRNA tested), or as not perturbed (i.e., the relative abundance is the same). In various embodiments, a difference between the two sources of RNA of at least a factor of about 25% (RNA from one source is 25% more abundant in one source than the other source), more usually about 50%, even more often by a factor of about 2 (twice as abundant), 3 (three times as abundant) or 5 (five times as abundant) is scored as a perturbation. Perturbations may be used by a computer for calculating and expression comparisons.

Preferably, in addition to identifying a perturbation as positive or negative, it is advantageous to determine the magnitude of the perturbation. This may be carried out, as noted above, by calculating the ratio of the emission of the two fluorophores used for differential labeling, or by analogous methods that will be readily apparent to those of skill in the art.

A computer readable medium may further comprise a pointer to a descriptor of a stage of lung cancer or to a treatment for lung cancer.

In operation, the means for receiving gene expression data, the means for comparing the gene expression data, the means for presenting, the means for normalizing, and the means for clustering within the context of the systems of the present invention may involve a programmed computer with the respective functionalities described herein, implemented in hardware or hardware and software; a logic circuit or other component of a programmed computer that performs the operations specifically identified herein, dictated by a computer program; or a computer memory encoded with executable instructions representing a computer program that may cause a computer to function in the particular fashion described herein.

Those skilled in the art will understand that the systems and methods of the present invention may be applied to a variety of systems, including IBM®-compatible personal computers running MS-DOS® or Microsoft Windows®.

The computer may have internal components linked to external components. The internal components may include a processor element interconnected with a main memory. The computer system may be an Intel Pentium®-based processor of 200 MHz or greater clock rate and with 32 MB or more of main memory. The external component may comprise a mass storage, which may be one or more hard disks (which are typically packaged together with the processor and memory). Such hard disks are typically of 1 GB or greater storage capacity. Other external components include a user interface device, which may be a monitor, together with an inputing device, which may be a “mouse”, or other graphic input devices, and/or a keyboard. A printing device may also be attached to the computer.

Typically, the computer system is also linked to a network link, which may be part of an Ethernet link to other local computer systems, remote computer systems, or wide area communication networks, such as the Internet. This network link allows the computer system to share data and processing tasks with other computer systems.

Loaded into memory during operation of this system are several software components, which are both standard in the art and special to the instant invention. These software components collectively cause the computer system to function according to the methods of this invention. These software components are typically stored on a mass storage. A software component represents the operating system, which is responsible for managing the computer system and its network interconnections. This operating system may be, for example, of the Microsoft Windows family, such as Windows 95, Windows 98, or Windows NT. A software component represents common languages and functions conveniently present on this system to assist programs implementing the methods specific to this invention. Many high or low level computer languages may be used to program the analytic methods of this invention. Instructions may be interpreted during run-time or compiled. Preferred languages include C/C++, and JAVA®. Most preferably, the methods of this invention are programmed in mathematical software packages which allow symbolic entry of equations and high-level specification of processing, including algorithms to be used, thereby freeing a user of the need to procedurally program individual equations or algorithms. Such packages include Matlab from Mathworks (Natick, Mass.), Mathematica from Wolfram Research (Champaign, Ill.), or S-Plus from Math Soft (Cambridge, Mass.). Accordingly, a software component represents the analytic methods of this invention as programmed in a procedural language or symbolic package. In a preferred embodiment, the computer system also contains a database comprising values representing levels of expression of one or more genes whose expression is characteristic of lung cancer. The database may contain one or more expression profiles of genes whose expression is characteristic of lung cancer in different cells.

In an exemplary implementation, to practice the methods of the present invention, a user first loads expression profile data into the computer system. These data may be directly entered by the user from a monitor and keyboard, or from other computer systems linked by a network connection, or on removable storage media such as a CD-ROM or floppy disk or through the network. Next the user causes execution of expression profile analysis software which performs the steps of comparing and, e.g., clustering co-varying genes into groups of genes.

In another exemplary implementation, expression profiles are compared using a method described in U.S. Pat. No. 6,203,987. A user first loads expression profile data into the computer system. Geneset profile definitions are loaded into the memory from the storage media or from a remote computer, preferably from a dynamic geneset database system, through the network. Next the user causes execution of projection software which performs the steps of converting expression profile to projected expression profiles. The projected expression profiles are then displayed.

In yet another exemplary implementation, a user first leads a projected profile into the memory. The user then causes the loading of a reference profile into the memory. Next, the user causes the execution of comparison software which performs the steps of objectively comparing the profiles.

10.4. Exemplary Diagnostic and Prognostic Compositions and Devices of the Invention

Any composition and device (e.g., a microarray) used in the above-described methods are within the scope of the invention.

In one embodiment, the invention provides compositions comprising a plurality of detection agents for detecting expression of genes in FIGS. 2, 3, and 4, or TrkB or Aur2. In a preferred embodiment, a composition comprises at least 2, preferably at least 3, 5, 10, 20, 50, or 100 different detection agents. A detection agent may be a nucleic acid probe, e.g., DNA or RNA, or it may be a polypeptide, e.g., as antibody that binds to the polypeptide encoded by a gene listed in FIGS. 2, 3, and 4, or TrkB or Aur2. The probes may be present in equal amount or in different amounts in the solution.

A nucleic acid probe may be at least about 10 nucleotides long, preferably at least about 15, 20, 25, 30, 50, 100 nucleotides or more, and may comprise the full length gene. Preferred probes are those that hybridize specifically to genes listed in FIGS. 2, 3, and 4, or TrkB or Aur2. If the nucleic acid is short (i.e., 20 nucleotides or less), the sequence is preferably perfectly complementary to the target gene (i.e., a gene that is involved in pathogenesis of lung cells), such that specific hybridization may be obtained. However, nucleic acids, even short ones, that are not perfectly complementary to the target gene may also be included in a composition of the invention, e.g., for use as a negative control. Certain compositions may also comprise nucleic acids that are complementary to, and capable of detecting, an allele of a gene.

In a preferred embodiment, the invention provides nucleic acids which hybridize under high stringency conditions of 0.2 to 1×SSC at 65° C. followed by a wash at 0.2×SSC at 65° C. to genes whose expression is characteristic of lung cancer. In another embodiment, the invention provides nucleic acids which hybridize under low stringency conditions of 6×SSC at room temperature followed by a wash at 2×SSC at room temperature. Other nucleic acids probes hybridize to their target in 3×SSC at 40 or 50° C., followed by a wash in 1 or 2×SSC at 20, 30, 40, 50, 60, or 65° C.

Nucleic acids which are at least about 80%, preferably at least about 90%, even more preferably at least about 95% and most preferably at least about 98% identical to genes involved in pathogenesis of lung cells or cDNAs thereof, and complements thereof, are also within the scope of the invention.

Nucleic acid probes may be obtained by, e.g., polymerase chain reaction (PCR) amplification of gene segments from genomic DNA, cDNA (e.g., by RT-PCR), or cloned sequences. PCR primers are chosen, based on the known sequence of the genes or cDNA, that result in amplification of unique fragments. Computer programs may be used in the design of primers with the required specificity and optimal amplification properties. See, e.g., Oligo version 5.0 (National Biosciences). Factors which apply to the design and selection of primers for amplification are described, for example, by Rylchik, W. (1993) “Selection of Primers for Polymerase Chain Reaction,” in Methods in Molecular Biology, Vol. 15, White B. ed., Humana Press, Totowa, N.J. Sequences may be obtained from GenBank or other public sources.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. ((1988) Nucl. Acids Res. 16: 3209), methylphosphonate oligonucleotides may be prepared by use of controlled pore glass polymer supports (Sarin et al., (1988) PNAS 85: 7448-7451), etc. In another embodiment, the oligonucleotide is a 2′-0-methylribonucleotide (Inoue et al., (1987) Nucl. Acids Res. 15: 6131-6148), or a chimeric RNA-DNA analog (Inoue et al., (1987) FEBS Lett. 215: 327-330).

Probes having sequences of genes listed in FIGS. 2, 3, and 4, or of TrkB or Aur2 may also be generated synthetically. Single-step assembly of a gene from large numbers of oligodeoxyribonucleotides may be done as described by Stemmer et al., Gene (Amsterdam) (1995) 164(1):49-53. In this method, assembly PCR (the synthesis of long DNA sequences from large numbers of oligodeoxyribonucleotides (oligos)) is described. The method is derived from DNA shuffling (Stemmer, (1994) Nature 370:389-391), and does not rely on DNA ligase, but instead relies on DNA polymerase to build increasingly longer DNA fragments during the assembly process. For example, a 1.1-kb fragment containing the TEM-1 beta-lactamase-encoding gene (bla) may be assembled in a single reaction from a total of 56 oligos, each 40 nucleotides (nt) in length. The synthetic gene may be PCR amplified and makes this approach a general method for the rapid and cost-effective synthesis of any gene.

“Rapid amplification of cDNA ends,” or RACE, is a PCR method that may be used for amplifying cDNAs from a number of different RNAs. The cDNAs may be ligated to an oligonucleotide linker and amplified by PCR using two primers. One primer may be based on sequence from the instant nucleic acids, for which full length sequence is desired, and a second primer may comprise a sequence that hybridizes to the oligonucleotide linker to amplify the cDNA. A description of this method is reported in PCT Pub. No. WO 97/19110.

In another embodiment, the invention provides compositions comprising a plurality of agents which may detect a polypeptide encoded by a gene involved in the pathogenesis of lung cells. An agent may be, e.g., an antibody. Antibodies to polypeptides described herein may be obtained commercially, or they may be produced according to methods known in the art.

The probes may be attached to a solid support, such as paper, membranes, filters, chips, pins or glass slides, or any other suitable substrate, such as those further described herein. For example, probes of genes involved in the pathogenesis of lung cells may be attached covalently or non covalently to membranes for use, e.g., in dot blots, or to solids such as to create arrays, e.g., microarrays.

10.5. Alternative Diagnostic Methods

In other embodiments of the diagnostic methods provided by the present invention, methods of diagnosis may comprise the steps of (a) determining the activity of a protein encoded by a gene selected from the panels of the invention in the lung cells of a subject, and (b) comparing the activity of said protein in said subject's cells with that in a normal lung cell of the same type. In certain embodiments, a particular type of lung cancer may be diagnosed if the protein whose activity is determined is associated with a particular type of lung cancer, such as adenocarcinoma or squamous cell carcinoma. Assays to determine the activity of a particular protein are routinely used in the art, are well-known to one of skill in the art, and may be adapted to the methods of the present invention with no more than routine experimentation.

11. Kits for Diagnosis and Prognosis of Lung Cancer

The invention further provides kits for determining the expression level of genes whose expression is characteristic of lung cancer. The kits may be useful for identifying subjects that are predisposed to developing a lung cancer or who have lung cancer, as well as for identifying and validating therapeutics for lung cancers. In one embodiment, the kit comprises a computer readable medium on which is stored one or more gene expression profiles of diseased cells of a subject having lung cancer, or at least values representing levels of expression of one or more genes whose expression is characteristic of lung cancer. The computer readable medium may also comprise gene expression profiles of counterpart normal cells, diseased cells treated with a drug, and any other gene expression profile described herein. The kit may comprise expression profile analysis software capable of being loaded into the memory of a computer system.

A kit may comprise suitable reagents for determining the level of protein activity in the lung cells of a subject.

A kit may comprise a microarray comprising probes of genes whose expression is characteristic of lung cancer. A kit may comprise one or more probes or primers for detecting the expression level of one or more genes whose expression is characteristic of lung cancer and/or a solid support on which probes attached and which may be used for detecting expression of one or more genes whose expression is characteristic of lung cancer in a sample. A kit may further comprise nucleic acid controls, buffers, or instructions for use.

Kit components may be packaged for either manual or partially or wholly automated practice of the foregoing methods. In other embodiments involving kits, this invention provides a kit including compositions of the present invention. The above-described kits may optionally contain instructions for their use. Such kits may have a variety of uses, including, for example, imaging, diagnosis, therapy.

Exemplification

The present invention is further illustrated by the following examples which should not be construed as limiting in any way. The contents of all cited references including literature references, issued patents, published or non published patent applications as cited throughout this application are hereby expressly incorporated by reference in their entireties. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. (See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986) (Cold Spring Harbor Laboratory Press, Cold Spring Harbor, N.Y., 1986); U.S. Pat. No. 5,830,645; U.S. Pat. No. 6,040,138; and U.S. Pat. No. 5,143,854.

### EXAMPLE 1

### Preparation of Tissue Samples for Microarray Analysis

A total of 39 tissue samples; 24 tumorous tissues comprising both adenocarcinoma and squamous cell carcinoma at all stages (occult, stage I-IV, and recurrent), one neuroendocrine tumor, one bronchiolalveolar, one large cell tumor, and 13 normal lung tissue samples were obtained from Dr. Ethan Dmitrovsky of Dartmouth Medical School. Of these samples, 8 were “matched-pairs”, in that for a given tumor tissue sample, normal tissue from the same individual was also obtained.

Total RNA was obtained from surgically resected lung tumor tissue samples or from cell lines. RNA samples were purified through CsCl gradients, phenol—chloroform extracted, and repurified on a Qiagen RNAeasy column according to manufacturer's recommendation. To verify the integrity of the isolated RNA, aliquots of each sample were electrophoresed on 1% denaturing agarose gels. Samples that exhibited an intact 28S and 18S ribosomal band were selected for generation of probes. The RNAs were prepared for Affymetrix microarray analysis using materials and methods provided by Affymetrix. Briefly, cDNAs of the total RNA were generated using T7-dT24 primer. Antisense c-RNA was generated using biotin labeled ribonucleotides and an in vitro transcription kit. The c-RNAs were fragmented and hybridized to the microarray overnight. The hybridized array was stained with SAPE (streptavidin-phycoerythrin). The hybridization levels (e.g., SAPE fluorescence) were measured using a Hewlett-Packard GeneArray® scanner.

### EXAMPLE 2

### Construction of Microarray

An excess of 10,000 individual genes (and or ESTs) were selected for inclusion on the microarray from the Incyte GeneAlbum database. These genes were selected based on the following criteria: 1) genes whose expression levels remain constant in normal tissues; 2) genes described in the literature to be involved in tumorgenesis in other cancers; 3) genes determined experimentally using microarrays to be differentially regulated between normal and other kinds of tumorous samples; 4) genes encoding proteins in the following protein families: protein kinases, protein phosphatases, proteases, nuclear hormone receptors; 5) genes determined to be differentially regulated by at least three fold using electronic subtraction of libraries generated from normal and tumor samples (libraries included lung, breast, colon, prostate); 6) genes exhibiting preferential expression in lung or bronchial epithelial cells relative to other organ tissues; 7) genes localized to chromosomal regions 3p, 9p, 12q, 15p, 15q, 17p, 19p, 20p, and 22q; 8) known genes implicated in transformation, carcinogenesis including oncogenes, tumor suppressors, signaling pathways, genes mapped to chromosomal regions amplified or deleted in tumors; 9) tumor-regulated: genes shown to be up or down regulated in tumors relative to non-tumor control tissue; and 10) genes exhibiting different tissue specificity; e.g., those restricted to expression in lung cells.

Sequences for each of the selected genes were provided to Affymetrix for selection of oligonucleotide probe sets. Before approving the set of probes, probe sequences selected by Affymetrix were counter-selected against a sequence to remove additional sequences that may cross react with other sequence of interest. The custom-made microarray had 8600 probes.

### EXAMPLE 3

### Gene Expression Analysis in Tissue Using Microarray

To this end, the custom microarray was interrogated individually with cRNAs derived from cellular RNA isolated from tumor and normal samples. Tumor and normal samples were categorized based on their histopathological diagnosis, i.e. normal, adenocarcinoma, squamous cellcarcinoma, etc. The Affymetrix GeneChip software, a proprietary software analysis system, was used to determine an average difference value for each gene. The average difference value was then used as the signal intensity for each gene. A database composed of signal intensities for the 8600 genes contained within the microarray and sample information was created. Thus, for each sample analyzed by the microarray, all 8600 signal intensities were captured in an organized and searchable format.

In order to identify candidate genes associated with or causing cancer, a series of analysis were performed using the signal intensity values obtained from the hybridization of normal and tumor samples. These methods utilized statistical algorithms, differential gene expression values obtained by comparing signal intensities from normal and tumor samples, and combining these methods with additional types of gene expression data and human genetic data. The methods are outlined below.

1. Common reference approach: All normal and tumor profiles were compared to one total, normal lung sample and the fold changes in expression were calculated. These values were used in a hierarchical clustering algorithm to analyze and group the samples based on the similarity of their differential gene expression patterns. The results of the hierarchical clustering demonstrated that there were three major groups of tumor samples: the squamous cell carcinoma samples formed one group, the adenocarcinoma samples formed a second cluster, and all other tumor types formed a third cluster. This analysis identified one normal sample as being more closely related to tumor samples and was omitted from further analysis. For subsequent analysis, signal intensities from normal samples were used to generate differential gene expression values comparing normal samples against individual tumor samples. To eliminate dependence of the analysis on any one reference sample, multiple reference sets of electronically pooled normal samples were generated. Three electronically pooled reference sets were created from the normal samples. The composition of these pools were determined from a multidimensional scaling (MDS) analysis of the normal samples and grouping sets of normal samples that exhibited the similar expression patterns as determined by the MDS distribution. The three reference sets chosen for use were termed Reference 1 (Ref1), Reference 2 (Ref2), and Reference 5 (Ref5). These electronically generated reference sets were compared individually against each tumor sample to determine fold difference.

2. Matched-pair sample approach: For eight individuals, for whom both tumor and normal adjacent tissue was available, the signal intensities of each gene could be compared for each matched-pair. This resulted in the identification of 1200 genes with greater than +/−3-fold differential expression in at least two out of the eight individuals.

3. Statistical approach: All normal tissue gene expression intensities were grouped into one bin and all tumor tissue gene expression were grouped into another bin. The student two-tailed t-test, unpaired with unequal variance was performed on all the data. These results were sorted by the lowest p-values and 160 genes with p<0.01 and 780 genes with p<0.05 were identified.

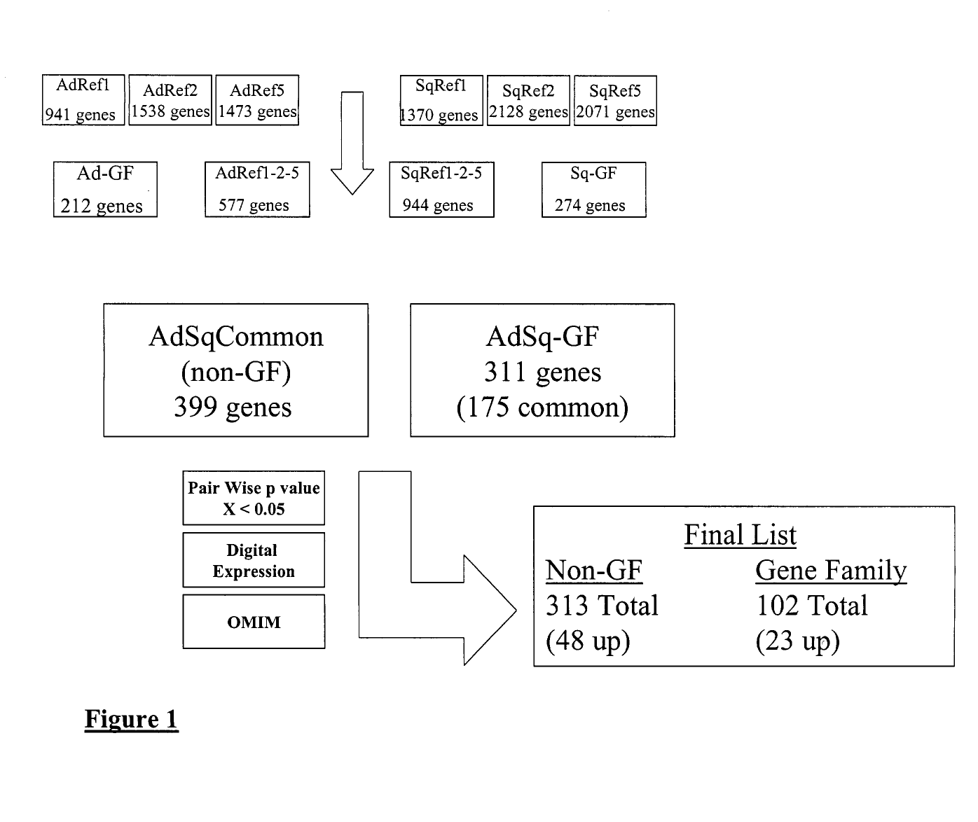

The results of each type of independent analysis were then compared to, or combined with each other to identify a set of genes identified by all reference sets as playing a role in the pathogenesis of lung cancer (e.g., observed across all tumor samples). The rounds of selection originating from these results are depicted in FIG. 1. The three common reference samples, Ref1, Ref2, and Ref5 were compared individually to each of the adenocarcinoma (Ad) samples and the resulting differential gene expression values were stored separately. Similarly, the three common references were compared to each of the squamous (Sq) cell carcinoma tumor samples. Genes that exhibited greater than +/−1.8 fold difference in gene expression in ⅔rds of each tumor type were selected. The gene totals for each set of comparisons can be seen in FIG. 1 (e.g., AdRef1 identified 941 genes).

Because more information is known for genes that fall into certain gene families, especially those gene families that are known to be targets of drugs, we segregated these gene family genes into a separate group. For example, all gene family genes identified within AdRef1, AdRef2, AdRef5, were combined to form Ad-GF (212 genes). The remaining genes identified by AdRef1, AdRef2, AdRef5 were condensed into a non-redundant set, AdRef1-2-5. A similar approach was utilized for the squamous cell carcinoma data.

To identify genes common to the AdRef1, AdRef2, AdRef5, SqRef1, SeRef2, SeRef5 sets, a commonality filter was applied to these data sets. A total of 399 genes common to all data Ad and Sq data sets were identified, AdSqCommon. Additionally, the Ad-GF and Sq-GF data sets were combined resulting in 311 non-redundant genes with 175 genes identified in common between the two data sets, AdSq-GF.

In order to assist in the selection of candidate genes as well as provide further evidence for selection, additional criteria were incorporated into the analysis. These additional criteria included (1) the statistical probabilities (p-values) obtained from the pair wise or matched pair comparisons, (2) other forms of RNA expression data, specifically digital expression data obtained from SAGE analysis (NCBI, CGAP) and transcript imaging data obtained from Incyte Genomics Inc., and (3) genetic and disease relevant information obtained from OMIM (Online Mendelian Inheritance in Man).

The genes comprising the panels of the invention are given in FIGS. 2-4 of the Detailed Description. FIG. 2 comprises genes that were differentially expressed in all types of lung cancers analyzed; FIG. 3 comprises genes that were differentially expressed in adenocarcinoma; and FIG. 4 comprises genes that were differentially expressed in squamous cell carcinoma.

### EXAMPLE 4

### Method for Correlating Gene Expression with Protein Expression

To illustrate that differential gene expression may correlates with protein expression in lung tumor tissue, TrkB-encoded protein expression was evaluated in lung tumor tissue. TrkB is a high affinity receptor for several members of the neurotrophin family. BDNF is considered to be the major ligand for TrkB, although NT3, 4 and 5 can also bind to this receptor. Ligand stimulation of TrkB leads to receptor homodimerization/conformational changes and the activation of the associated kinase. The Trk family includes TrkA, TrkB, and TrkC, which are highly homologous in the intracelluar domains. For example, TrkA and TrkB vary only by a single amino acid at close proximity to the ATP-binding pocket.

Trk receptors are expressed in a number of neuroendocrine-derived tissues. In adults, high level expression of TrkB appears to be restricted to neuronal tissues. Most hippocampal and motor neurons express TrkB. These same neurons also express TrkA and TrkC. BDNF, which stimulates TrkB, but not A or C, is thought to act as a survival factor in the brain. The blood-brain barrier may prevent access to the brain (the mostly likely tissue to be adversely affected by TrkB inhibition), thus a lung cancer therapeutic directed toward the TrkB gene or gene product would likely have few side effects.

Antibodies to TrkB were used to determine protein over-expression in lung cancer.

Data from antibody staining:

Stained 6 paraffin blocks: 4 Squamous, 2 adenocarcinomas are positive. No staining in adjacent normal tissue.

Strong staining in 100% of 33 paraffin-embedded tumor tissues Squamous (10) Adeno (8) Large cell (7) Bronchioalveolar (4) Small cell (3)

Strong tumor-specific staining in frozen lung tumor tissue-100% of samples Adeno (3) Squamous (3) large cell (2) Neurodendocrine (1)—most intense.

As supported by the pattern of antibody staining, the compositions and methods of the present invention permit the identification of proteins expressed in lung cancer cells.

### REFERENCES

The contents of all cited references including literature references, issued patents, published or non published patent applications cited throughout this application as well as those listed below are hereby expressly incorporated by reference in their entireties. In case of conflict, the present application, including any definitions herein, will control.

Equivalents

The invention now being fully described, it will be apparent to one of ordinary skill in the art that many changes and modifications may be made thereto without requiring more than routine experimentation or departing from the spirit or scope of the appendant claims.

The specification, including the appendant claims and examples should be considered exemplary only with the true scope and spirit of the invention suggested by the following claims.

We claim:

1. A method for identifying a candidate therapeutic for lung cancer comprising contacting a compound with a protein encoded by a gene selected from the panel of genes listed in FIG. 2, wherein binding indicates a candidate therapeutic.

2. The method of claim 1 wherein said compounds are selected from the following classes of compounds: proteins, peptides, peptidomimetics, and small molecules.

3. The method of claim 1, wherein said cancer is adenocarcinoma.

4. The method of claim 1, wherein said cancer is squamous cell carcinoma.

5. The method of claim 1, wherein said compound is in a library of compounds.

6. The method of claim 1, wherein said library is generated using combinatorial synthetic methods.

7. The method of claim 1, wherein binding is determined using an in vitro assay.

8. The method of claim 1, wherein binding is determined using an in vivo assay.

9. The method of claim 1, wherein said protein is encoded by TrkB.

10. The method of claim 1, wherein said protein is encoded by Aur2.

11. A method for identifying a candidate therapeutic for adenocarcinoma comprising contacting a compound with a protein encoded by a gene selected from the panel of genes listed in FIG. 3, wherein binding indicates a candidate therapeutic.

12. A method for identifying a candidate therapeutic for squamous cell carcinoma comprising contacting a compound with a protein encoded by a gene selected from the panel of genes listed in FIG. 4, wherein binding indicates a candidate therapeutic.

13. A method for identifying a candidate therapeutic for lung cancer comprising contacting a compound with a gene selected from the panel of genes listed in FIG. 2, wherein binding indicates a candidate therapeutic.

14. The method of claim 13, wherein said compounds of said library are selected from: antisense nucleic acids, small molecules, polypeptides, proteins, peptidomimetics, and nucleic acid analogs

15. The method of claim 13, wherein said cancer is adenocarcinoma.

16. The method of claim 13, wherein said cancer is squamous cell carcinoma.

17. The method of claim 13, wherein said compound is in a library of compounds.

18. The method of claim 13, wherein said library is generated using combinatorial synthetic methods.

19. The method of claim 13, wherein said binding assay is in vitro.

20. The method of claim 13, wherein said binding assay is in vivo.

21. The method of claim 13, wherein said gene is TrkB.

22. The method of claim 13, wherein said gene is Aur2.

23. A method for identifying a candidate therapeutic for adenocarcinoma comprising contacting a compound with a gene selected from the panel of genes listed in FIG. 3, wherein binding indicates a candidate therapeutic.

24. A method for identifying a candidate therapeutic for squamous cell carcinoma comprising contacting compounds with a gene selected from the panel of genes listed in FIG. 4, wherein binding indicates a candidate therapeutic.

25. A method for identifying a candidate therapeutic for lung cancer comprising contacting a compound with a gene that is differentially regulated during neoplasia selected from the panel consisting of the genes listed in FIG. 2, wherein the expression of said gene is normalized.

26. The method of claim 25, wherein said gene is selected from the panel consisting of the genes listed in FIG. 3.

27. The method of claim 25, wherein said gene is selected from the panel consisting of the genes listed in FIG. 4.

28. The method of claim 25, wherein said gene is TrkB.

29. The method of claim 25, wherein said gene is Aur2.

30. A method for identifying a candidate therapeutic for lung cancer comprising contacting a compound with a protein whose activity promotes neoplasia encoded by a gene selected from the panel consisting of the genes listed in FIG. 2, wherein the ability to inhibit the protein's activity indicates a candidate therapeutic.

31. The method of claim 30, wherein said gene is selected from the panel consisting of the genes listed in FIG. 3.

32. The method of claim 30, wherein said gene is selected from the panel consisting of the genes listed in FIG. 4.

33. The method of claim 30, wherein said gene is TrkB.

34. The method of claim 30, wherein said gene is Aur2.

35. A method for identifying a candidate therapeutic for treating lung cancer, comprising comparing the expression profile of a cell incubated with a test compound, wherein the cell is essentially identical to the normal counterpart cell of a diseased lung cell, with the expression profile of a normal counterpart cell of a diseased lung cell, wherein a similar expression profile in the two cells indicates that the compound is likely to be effective as a therapeutic for lung cancer.

36. A method for determining the efficacy of a candidate therapeutic as a drug for lung cancer comprising the steps of:
a) contacting a candidate therapeutic to a lung tumor cell of a subject, and
b) determining the ability of said candidate therapeutic to inhibit pathogenesis of the cell.

37. A method for determining the efficacy of a candidate therapeutic as a drug for lung cancer comprising the steps of:
a) contacting a candidate therapeutic to a lung tumor cell of a subject, and
b) determining the ability of said candidate therapeutic to normalize the expression profile of said cell.

38. A pharmaceutical composition, comprising: a therapeutic amount of an agent identified using any of the methods of claims 1-37, and a pharmaceutically-acceptable carrier, vehicle, excipient, or diluent.

39. A method for treating a subject that has lung cancer, comprising administering a therapeutically-effective amount of a pharmaceutical composition to said subject to normalize the expression of a gene or group of genes selected from the genes listed in FIG. 2, wherein said expression levels of said subject's genes are returned to those of a normal subject.

40. The method of claim 39, wherein the gene is TrkB.

41. The method of claim 39, wherein the gene is Aur2.

42. The method of claim 39, wherein said subject has adenocarcinoma and the genes are selected from FIG. 3.

43. The method of claim 39, wherein said subject has squamous cell carcinoma and the genes are selected from FIG. 4.

44. A method for treating a subject that has lung cancer, comprising administering a therapeutically-effective amount of a pharmaceutical composition to said subject to inhibit the activity of a protein encoded by a gene selected from the genes listed in FIG. 2.

45. The method of claim 44, wherein the protein is encoded by TrkB.

46. The method of claim 44, wherein the protein is encoded by Aur2.

47. The method of claim 44, wherein said subject has adenocarcinoma and the genes are selected from FIG. 3.

48. The method of claim 44, wherein said subject has squamous cell carcinoma and the genes are selected from FIG. 4.

49. A method for treating a subject that has lung cancer, comprising administering a therapeutically-effective amount of protein encoded by a gene selected from the genes listed in FIG. 2.

50. The method of claim 49, wherein the protein is encoded by TrkB.

51. The method of claim 49, wherein the protein is encoded by Aur2.

52. The method of claim 49, wherein said gene is selected from FIG. 3.

53. The method of claim 49, wherein said gene is selected from FIG. 4.

54. A method of cancer chemoprevention including any of the methods of claims 39-53, wherein said subject has had lung cancer or is at risk for lung cancer and said method is used in preventative treatment.

55. A kit for treating a patient with lung cancer, comprising any of the therapeutic agents identified by any of the methods of claims 1-53, formulated in a pharmaceutically-acceptable carrier, vehicle, excipient, or diluent, and optionally including instructions for use.

56. A composition comprising a plurality of detection agents of genes whose expression is characteristic of lung cancer, and which are capable of detecting the expression of the genes or the polypeptide encoded by the genes.

57. The composition of claim 56, wherein the detection agents are isolated nucleic acids which hybridize specifically to nucleic acids corresponding to the genes whose expression is characteristic of lung cancer.

58. The composition of claim 57, comprising isolated nucleic acids which hybridize specifically to genes listed in FIG. 2.

59. The composition of claim 57, comprising isolated nucleic acids which hybridize specifically to genes listed in FIG. 3.

60. The composition of claim 57, comprising isolated nucleic acids which hybridize specifically to genes listed in FIG. 4.

61. The composition of claim 58, comprising isolated nucleic acids which hybridize specifically to at least 10 different nucleic acids corresponding to genes whose expression is characteristic of lung cancer.

62. The composition of claim 58, comprising isolated nucleic acids which hybridize specifically to at least 100 different nucleic acids corresponding to genes whose expression is characteristic of lung cancer.

63. The composition of claim 58, comprising isolated nucleic acids which hybridize to essentially all the genes listed in FIG. 2.

64. The composition of claim 56, wherein the detection agents detect the polypeptides encoded by the genes whose expression is characteristic of lung cancer.

65. The composition of claim 64, wherein the detection agents are antibodies reacting specifically with the polypeptides.

66. A solid surface to which are linked a plurality of detection agents of genes whose expression is characteristic of lung cancer, and which are capable of detecting the expression of the genes or the polypeptide encoded by the genes.

67. The solid surface of claim 66, wherein the detection agents are isolated nucleic acids which hybridize specifically to nucleic acids corresponding to the genes whose expression is characteristic of lung cancer.

68. The solid surface of claim 67, comprising isolated nucleic acids which hybridize specifically to genes listed in FIG. 2.

69. The solid surface of claim 67, comprising isolated nucleic acids which hybridize specifically to genes listed in FIG. 3.

70. The solid surface of claim 67, comprising isolated nucleic acids which hybridize specifically to genes listed in FIG. 4.

71. The solid surface of claim 68, comprising isolated nucleic acids which hybridize specifically to at least 10 different nucleic acids corresponding to genes whose expression is characteristic of lung cancer.

72. The solid surface of claim 71, comprising nucleic acids which hybridize specifically to at least 100 different nucleic acids corresponding to genes whose expression is characteristic of lung cancer.

73. The solid surface of claim 72, comprising isolated nucleic acids which hybridize to essentially all of the genes listed in FIG. 2.

74. The solid surface of claim 66, wherein the detection agents detect the polypeptides encoded by the genes whose expression is characteristic of lung cancer.

75. The solid surface of claim 74, wherein the detection agents are antibodies reacting specifically with the polypeptides.

76. The solid surface of claim 66, wherein the detection agents are covalently linked to the solid surface.

77. The solid surface of claim 76, wherein the solid surface is a microarray.

78. A composition comprising agonists and/or antagonists of a plurality of genes whose expression is characteristic of lung cancer.

79. The composition of claim 78, wherein the agonists are polypeptides encoded by the genes or functional fragments or equivalents thereof.

80. The composition of claim 79, comprising at least one polypeptide or functional fragment or equivalent of a polypeptide selected from the group consisting of polypeptides encoded by the genes listed in FIG. 2.

81. The composition of claim 79, comprising at least one polypeptide or functional fragment or equivalent of a polypeptide selected from the group consisting of polypeptides encoded by the genes listed in FIG. 3.

82. The composition of claim 79, comprising at least one polypeptide or functional fragment or equivalent of a polypeptide selected from the group consisting of polypeptides encoded by the genes listed in FIG. 4.

83. The composition of claim 78, wherein the agonists are isolated nucleic acids encoding the polypeptides or functional fragments or equivalents thereof that are encoded by genes whose expression is characteristic of lung cancer.

84. The composition of claim 78, wherein the antagonists are antisense nucleic acids or siRNAs.

85. A method for comparing a level of expression of at least one gene whose expression is characteristic of lung cancer in a subject and at least one level of expression of a set of reference levels of expression, comprising
a) providing nucleic acids from a cell of a subject, the cell being of the same type as that of a diseased lung cell,
b) determining the level of expression of at least one gene whose expression is characteristic of lung cancer, and
c) comparing the level of expression of the at least one gene from a cell of the subject at least one level of expression of a set of reference levels of expression,
to thereby compare the level of expression of at least one gene whose expression is characteristic of lung cancer in the subject with at least one level of expression of a set of reference levels of expression.

86. The method of claim 85, wherein the set of reference expression levels includes the level of expression of at least one gene whose expression is characteristic of lung cancer in a subject having lung cancer.

87. The method of claim 85, comprising determining the level of expression of at least one gene selected from the panel consisting of the genes listed in FIG. 2.

88. The method of claim 85, comprising determining the level of expression of at least one gene selected from the panel consisting of the genes listed in FIG. 3.

89. The method of claim 85, comprising determining the level of expression of at least one gene selected from the panel consisting of the genes listed in FIG. 4.

90. The method of claim 85, comprising incubating a nucleic acid sample derived from the RNA of the cell of the subject with a nucleic acid corresponding to at least one gene whose expression is characteristic of lung cancer, under conditions wherein two complementary nucleic acids hybridize to each other.

91. The method of claim 85, wherein the at least one nucleic acid corresponding to at least one gene whose expression is characteristic of lung cancer is attached to a solid surface.

92. The method of claim 91, wherein the solid surface is a microarray.

93. The method of claim 85, comprising entering the level of expression of at least one gene into a computer comprising a memory with values representing the level of expression of the at least one gene in the set of reference expression levels.

94. The method of claim 93, wherein comparing the level comprises providing computer instructions to perform.

95. The method of claim 85, wherein a set of reference expression levels includes the level of expression of one or more genes whose expression is characteristic of lung cancer in a subject having lung cancer.

96. The method of claim 95, wherein the set of reference expression levels further includes the level of expression of one or more genes whose expression is characteristic of lung cancer in a normal counterpart cell of a diseased lung cell.

97. The method of claim 95, for determining whether the subject has or is likely to develop lung cancer.

98. The method of claim 85, further comprising iteratively providing nucleic acid and determining the level of nucleic acid, such as to determine an evolution of the level of expression of the genes whose expression is characteristic of lung cancer in the subject.

99. The method of claim 98, wherein the subject is being treated for lung cancer and the method provides an evaluation of the efficacy of the treatment.

100. A method for determining whether a subject has or is likely to develop lung cancer, comprising:
a) determining a level of expression of at least one gene whose expression is characteristic of lung cancer in a cell of the subject, and
b) comparing the level of expression of the at least one gene with the level of expression of the at least one gene in a cell of a subject known to have lung cancer.
wherein a similar level of expression of the genes in the subject and in the subject known to have lung cancer indicates that the subject is likely to have or to develop lung cancer.

101. The method of claim 100, wherein the cell is a diseased lung cell.

102. The method of claim 100, wherein the level of expression of the at least one gene in a cell of a subject known to have lung cancer is in the form of a database.

103. The method of claim 102, wherein the database is included in a computer-readable medium.

104. The method of claim 103, wherein the database is in communications with a microprocessor and microprocessor instructions for providing a user interface to receive expression level data of a subject and to compare the expression level data with the database.

105. A method of diagnosing lung cancer comprising the steps of
a) determining the activity of a protein encoded by a gene selected from the panel of genes listed in FIG. 2 in the lung cells of a subject, and
b) comparing the activity of said protein in said subject's cells with that of a normal lung cell of the same type,
wherein a decreased or increased level of protein activity relative to a normal cell indicates that the subject may have lung cancer.

106. The method of claim 105, wherein the protein is encoded by a gene selected from the panel of genes listed in FIG. 3, and a decreased or increased level of protein activity relative to a normal cell indicates that the subject may have adenocarcinoma.

107. The method of claim 105, wherein the protein is encoded by a gene selected from the panel of genes listed in FIG. 4, and a decreased or increased level of protein activity relative to a normal cell indicates that the subject may have squamous cell carcinoma.

108. A method for selecting a therapy for a patient having lung cancer, comprising:
a) providing at least one query value corresponding to the level of expression of at least one gene whose expression is characteristic of lung cancer from a patient having lung cancer,
b) providing a plurality of sets of reference values corresponding to levels of expression of at least one gene whose expression is characteristic of lung cancer, each reference value being associated with a therapy, and
c) selecting the reference values most similar to the query values, to thereby select a therapy for said patient.

109. The method of claim 108, wherein selecting further includes weighing a comparison value for the reference values using a weight value associated with each reference values.

110. The method of claim 109, further comprising administering the therapy to the patient.

111. The method of claim 108, wherein the query values and the sets of reference values are expression profiles.

112. A method for selecting a therapy for a patient having lung cancer, comprising:
a) providing a plurality of reference expression profiles, each associated with a therapy,
b) providing a labeled target nucleic acid sample prepared from RNA of a diseased lung cell of the patient,
c) contacting the labeled target nucleic acid sample with an array comprising probes corresponding to essentially all the genes whose expression is characteristic of lung cancer to obtain an expression profile of the patient, and
d) selecting the reference profile most similar to the expression profile of the patient, to thereby select a therapy for the patient.

113. A method for selecting a therapy for a patient, comprising:
a) obtaining a patient sample,
b) identifying a subject expression profile of genes whose expression is characteristic of lung cancer from the patient sample,
c) selecting from a plurality of reference expression profiles a matching reference profile most similar to the subject expression profile, wherein the reference profiles and the subject expression profile have a plurality of values, each value representing the expression level of genes whose expression is characteristic of lung cancer in a particular cell, and wherein each reference profile is associated with a therapy, and
d) transmitting a descriptor of the therapy associated with the matching reference profile, thereby selecting a therapy for said patient.

114. The method of claim 113, further comprising receiving information about the outcome of the patient after the therapy is administered to the patient.

115. The method of claim 114, wherein the descriptor is transmitted across a network.

116. A kit for evaluating a drug, comprising an array comprising a plurality of addresses, wherein each address has disposed thereon at least one capture probe that hybridizes to at least one gene whose expression is characteristic of lung cancer.

117. The kit of claim 116, wherein the array comprises capture probes for essentially all the genes whose expression is characteristic of lung cancer selected from the panel of genes listed in FIG. 2.

118. The kit of claim 116, wherein the array comprises capture probes for essentially all the genes whose expression is characteristic of adenocarcinoma selected from the panel of genes listed in FIG. 3.

119. The kit of claim 116, wherein the array comprises capture probes for essentially all the genes whose expression is characteristic of squamous cell carcinoma selected from the panel of genes listed in FIG. 4.

120. A kit for evaluating a drug, comprising a computer-readable medium having a plurality of digitally-encoded expression profiles wherein each profile of the plurality has a plurality of values, each value representing the level of expression of a gene whose expression is characteristic of lung cancer in a particular cell.

121. A computer-readable medium comprising at least one digitally encoded value representing a level of expression of at least one gene whose expression is characteristic of lung cancer in a diseased cell.

122. The computer-readable medium of claim 121, comprising at least one value representing the level of expression of at last one gene selected from FIG. 2 in a diseased lung cell.

123. The computer-readable medium of claim 121, comprising at least one value representing the level of expression of at last one gene selected from FIG. 3 in a diseased cell of adenocarcinoma.

124. The computer-readable medium of claim 121, comprising at least one value representing the level of expression of at last one gene selected from FIG. 4 in a diseased cell of squamous cell carcinoma.

125. A computer-readable medium comprising at least one value representing a ratio between a level of expression of a gene whose expression is characteristic of lung cancer in a diseased cell and a level of expression of the gene in a normal counterpart cell of the diseased cell.

126. A computer-readable medium comprising at least one digitally encoded expression profile, comprising a plurality of values, each value representing a level of expression of a gene whose expression is characteristic of lung cancer in a diseased cell.

127. A computer-readable medium comprising a plurality of digitally-encoded expression profiles, wherein each profile of the plurality has a plurality of values, each value representing a level of expression of one or more genes whose expression is characteristic of lung cancer in a particular cell.

128. The computer-readable medium of claim 127, wherein each profile of the plurality is associated with a stage of lung cancer.

129. The computer-readable medium of claim 127, wherein each profile of the plurality is associated with a therapeutic treatment.

130. A computer system, comprising:
a) a database having at least one value representing a level of expression of at least one gene whose expression is characteristic of lung cancer in a diseased cell, and
b) a processor having instructions to receive at least one query value representing at least one level of expression of at least one gene whose expression is characteristic of lung cancer, and compare at least one query value and at least one database value.

131. A computer system according to claim 130, wherein the instructions to receive include instructions to provide a user interface.

132. A computer system according to claim 131, wherein the instructions further include instructions to display at least one comparison.

133. A computer system according to claim 130, wherein the instructions further include instructions to create at least one record based on the comparison.

134. A computer system according to claim 133, further including instructions to display at least one record.

135. A computer system according to claim 130, wherein the database values include essentially all of the values set forth in FIG. 2, FIG. 3, or FIG. 4.

136. The computer system of claim 130, wherein the database comprises at least one expression profile comprising a plurality of values, each value representing a level of expression of a gene whose expression is characteristic of lung cancer in a diseased cell.

137. A computer program for analyzing levels of expression of at least one gene whose expression is characteristic of lung cancer in a subject, the computer program being disposed on a computer readable medium and including instructions for causing a processor to:
a) receive at least one query value representing a level of expression of at least one gene whose expression is characteristic of lung cancer in a subject, and,
b) compare the at least one query value and at least one level of expression value, the at least one level of expression value representing at least one level of expression of at least one gene whose expression is characteristic of lung cancer in a diseased cell.

138. A computer program of claim 137, further comprising instructions to display at least one comparison.

139. A computer program of claim 137, wherein the instructions to compare include instructions to retrieve at least one level expression value from a computer readable medium.

140. A computer program of claim 137, where the instructions to compare include instructions to retrieve the at least one level expression value from a database.

141. A computer program of claim 137, wherein the instructions to receive include instructions to provide a user interface.

142. A computer program for analyzing an expression profile of a diseased lung cell in a subject, the computer program being disposed on a computer readable medium and including instructions for causing a processor to:
a) receive at least one query expression profiles comprising a plurality of values, each value representing a level of expression of a gene whose expression is characteristic of lung cancer in a diseased cell, and
b) compare the at least one query expression profile and at least one reference expression profile comprising a plurality of values, each value representing a level of expression of a gene whose expression is characteristic of lung cancer in a particular cell.
